SEQUENCE LISTING (174 sequences)

SEQ ID NO: 1 - DNA, 692 bp, unknown
  b1nr013, upregulated sequence from beech
acaaacctacaaacaagcatttaatacacacacagatatatatatgccacagtgacacctagtggcgtccaggaagcttctccttgatcttttccatgactcccttcttctcagcttgttgctcatggccctgaagattgtaaccaggcaaggtagttgaagttgtatgatcttggtgatgctgatcaccctggaccttgtccttgttcccagcacctggaatcttctcaaagaccttgtccatgcacccttgcccctgcttctcaggttgttgcccatggccctgagcattgtgaccaggcaaggtagttgaagttgtatgatcttggcgatactgatcaccctggaccttgtccttgttcccaacacctggaatcttctcaaagaccttgtccatggcccccttcttctcaggttgttgcccatggccctgagcattgtaaccaggcgtggtagttgaagttgtatgatcttggcgatactgatcaccctggactctgtccttgttcccagcacctggagtcttctcaaagaacttgttcatggcccccttcttctcagttgttgcccacctcgggcgccgaccacgctaatcgaattctcgcggccgccatggcggccggaacatgcgacgtccggccaaatcgccctatagtgagtcgtatacaattcctggcgtccttttaaaaaaa

SEQ ID NO: 2 - DNA, 284 bp, unknown
  b1nr031, upregulated sequence from beech
tagcggccgcccgggcaggtggcagggagagaccgttattcctggtggaactggtgggaagagccttgaagctcaggagcaccttgctgaaggacggagccatggagggcaggctaggaaggagcagctgggacatgaaggctaccaggagttgggcagcaagggagggcaggctaggaaggagcagctgggacatgaaggccaccaagagttgggcagcaaaggaggacaggctaggaaagagcagataggacatgaggggtacctcggccgcgaccacgcta

SEQ ID NO: 3 - DNA, 296 bp, unknown
  b1nr039, upregulated sequence from beech
tagcgtggtcgcggccgaggtacccttgacaacaaggcatcatagtctccttatgggcgatgtccaacaccaccttgcccatctctacgtccctgaccctgtcccccttcttgaccagggcctccttgttgtcctggtccttgagcaccaggaccaccaggaccacgcccatctttttgtccctgaccttgaccctgtccaccaagctgaccttgaccctgtccaccgagctgtccttgaccttcaccacaaccaggcccgccaggacgtcctacctgcccgggcggccgctcgaa

SEQ ID NO: 4 - DNA, 293 bp, unknown
  b4nr049, upregulated sequence from beech
tcgagcggccgcccgggcaggtacccttgacaacaaggcatcatagtctccttatgggcgatgtccaccaccaccttgcccatctctacgtccctgaccctatccaccttcttgaccagggcctccttgttgtcctggtccttgagcaccaggaccaccaggaccacgcccatctttttgtccctgaccttgaccctgtccaccaagctgaccttgaccctgtccaccgagctgtccttgaccttcaccacaaccaggcccgccaggacgtcacctcggccgcgaccacgcta

SEQ ID NO: 5 - DNA, 396 bp, unknown
  b4nr081, upregulated sequence from beech
tagcgtggtcgcggccgaggtacaagctttttttttttttttttttttttttttttccaaccaaaatttcccttttttttttaaatttcccccccccttttccccaaaaaaaggggcccctccttttttaaaaaaacccaatttttaaaaactttttttgcccttcccctttccccttttttttaaaatttaaaaaaaaattttatttttttttccctaaaaaaaccccccccccccctttttttttttttttccccccccacccaaaaaaaaaaatttttcccctttttttttttaaaacccaaccccttttttttttaaaaaccaaaaaaggcccccaaaaggggcctcccccaattttccccaggtttttttttttttttcccccccgggggg

SEQ ID NO: 6 - DNA, 352 bp, unknown
  b4nr096, upregulated sequence from beech
tagcgtggtcgcggccgaggtacgggactattgagtaactctggctaggcccttctgtggtgcctcttctggttttgtagaataaagtggcttaggttagtagttatgtaaaggtaatatgttgttttctggtttgtgcttgtattccatcagtgtgtgtgtgtatctgatggataaactagttaatgtttttgttgaagtctagctatagctggaatgggacagtgcatgatatggtcttagtatgttggtttttttatcatgcgtagttccctctttttgtgggtaatggttggctgtaatctaatagatgatgctgacttttggttgaaaaaaaaaaaaaaaaaaaaaa

SEQ ID NO: 7 - DNA, 508 bp, unknown
  b5nr012, upregulated sequence from beech
ttcgagcggccgcccgggcaggtacaaggatgaagctgcgcgaaatgaagcttataggggtaatgcggatgttgggcatgtaggaagagaaatagagaagattgaggtgaatattgaaaagacccgaccaggagctgtggctgctacactgaaagcagctgatgagatggctggtcagactttcaatgatgtgggacccttagatgaagagggcgtaattggtttggaccgtcaccgcaagatgtgattacgtccacctagcttcctaagagtggtttttttacatcatgatgatggtgttttcttttgctatcgttttctgtattttgttactgtgttatatagtccttactttttgagtccgacggactcgttatcttgttgctggaagtgaattttggataatatgaatatgatctcatagaatacaaaatggatagggcctgattttattcaggccaggtccatcttcgagttttatttggacacctcggccgcgaccacgcta

SEQ ID NO: 8 - DNA, 321 bp, unknown
  b5nr018, upregulated sequence from beech
ttcgagcggccgcccgggcaggtacaacgtaaggggcgctttaaggtcacttcggcagaccttagtcccaagggtcctacaaactgcatgtttaacccaatttctggatgttcaacgagtccaaccaccccaaaccttacggctgcctcaattctcccatctctgcaatttattttgcaacagaactccacacaaagggaagaacttatgaaattgatcaagtatgtggaacaaacctctggcaagcatgctgagtcagctgaggttgtcacaagtgacctgttgcagatgcctcctggtacctcggccgcgaccacgcta

SEQ ID NO: 9 - DNA, 405 bp, unknown
  b5nr019, upregulated sequence from beech
ttcgagcggccgcccgggcaggttttttaaagcctaaattactctctgtgttttgagttctaagtagaaactgaagatggacaaatcccagaatacgtgtttccaagctggccaggccaagggccaaactcaggaaaaggcaagcaacttgatggaaaaggctggaaatgctggtcagtctgccaaggaaacatgcctagaggctggtaagcaagtgccggctaaggcacaaggggctgctaatactgctaaggatgcagttggacttggagcaaagaaatgaaactaatattggacctaataaagccagctctccccaaataaattttgtaatttaatttttttttttccttttaagttatggattcttgttttctactttgtacctcggccgcgaccacgcta

SEQ ID NO: 10 - DNA, 642 bp, unknown
  b5nr052, upregulated sequence from beech
ttcgagcggccgcccgggcaggtgtttttgttttcaatgcttggagatgagtgcgagctaagcttcgctgttattagagatgtgctctgtcaatgtggatatgatgttgaaaaggccttggatgtgttgcttgaattatcggcttccacctttgagcagtccaggaatgatggttacctcaacaatagtgttaactataaagaagatataagatttcttattgaacgcaaagactttattaatgatggggcttccgattgcgcttcttattcttctgaaagtgaattccaggataatatatggtctctgggatatgggtgcaggaattatgcaaaggttcttgataatcctgaagctaattctccaactagcccaacaattactgtgtcagagctccctcaaaaggtgttggagtctttgtttaaagtccccaagagtcctcattatgagccaaccacaatgaattggaggaatgtagcaaagaaactgcagtctttggggcctgggtttgatgcttgttcttctagtgttgcggaaccccagcaaaatacttgcgctaaaggagaggaatatcatgtgttcagaaaatcttctaagcaacattgggattcaatgaagtctagctatcagaaagctgcagtg

SEQ ID NO: 11 - DNA, 385 bp, unknown
  b5nr078, upregulated sequence from beech
tagcgtggtcgcggccgaggtcaggaaattgattgtggaggtttgtgcaagcagaggtgcagtcttcattcgagaccaaacttgtgcaacagggcgtgcggcacatgctgtgtgaggtgcaagtgtgtgccacctgggactgctgggaacagagagctgtgtggggcatgctacactgaaatgaccacccatgggaacaagaccaaatgtccataaggcccaatggcaatggtggcggcccagattagctagctttggatccatgttctctgtgcctgtcaagcttttgttatgttgtgtgtttgtaatcaataatgtctgtctaaaatctaaatatagctgttatgtttctaaagccnaaaaaaaaaaaaaaaaaaaaaaaaaa

SEQ ID NO: 12 - DNA, 411 bp, unknown
  b6nr008, upregulated sequence from beech
ttcgagcggccgcccgggcaggtacccttgacaacaaggcatcatagtctccttatgggcgatgtccaacaccaccttgcccatctctacgtccctgaccctgtcccccttcttgaccagggcctccttgttgtcctggtccttgagcaccaggaccaccaggaccacgcccatctttttgtccctgaccttgaccctgtccaccaagctgaccttgaccctgtccaccgagctgtccttgaccttcaccacaaccaggcccgccaggacgtccttctcctcctagaccttgaccaccttggcgtccctcaccatgaggtgggttcccttgaggtccttggtgagacattatcctaagctttacttggaaattagagtattgaatacaagacctcggccgcgaccacgcta

SEQ ID NO: 13 - DNA, 112 bp, unknown
  b6nr046, upregulated sequence from beech
tcgagcggccgcccgggcaggtactctcttttgagctggttgaatttgtcttttgaattaacaagggagatcttctatagaagaaaaaaaaaaaaaaaaaaaaaaaaaaaaa

SEQ ID NO: 14 - DNA, 473 bp, unknown
  b6nr057, upregulated sequence from beech
tcgagcggccgcccgggcaggtaccagagaggatcaacggtaggctagcaatgattggctttgttgcagcgttgggacaggagttctatactggccaggatgtgtttgcccaaatagccaatggtggaatcccatggttcctagcaactagcattctgctatcttctgcatctctgattcctttgtttaaaggtatcagcgcagagtccaagtcagatggggtgatgacctcagatgctgagatgtggaatgggaggtttgccatgttgggtttggttgcactggctgttaccgaatatgtgaagggtggaacccttatatagatctctgatttggtaacacaggcagtccaacttgtgtagataaaaagtatactaataatttccatgtaattcggaaagaaactcatgtataatgtatttcaattttcagtaaatcaaatttcaattttaccaaaaaaaaaaaaaaaaaaa

SEQ ID NO: 15 - DNA, 600 bp, unknown
  b6nr061, upregulated sequence from beech
tagcgtggtcgcggccgaggtttttttttttttttttttttggggaaaaatgaaattttgttttcttaaaattgaaaatactttttacttaatttttttcccaattaactggaaattttttattttattttttttcaacaaaattggaattgctttttttcccaattaaaaattttttttagggttcccccccttcaatttttgggtaacacccgttgaaacaaaccccaaattggaaaacccccctttccaattttaagtttttggggtcttccccccttttaatttgaattttgccctattccttttaaacaagggattcaaaattccaaaaaataccaaattgtttttttttagaaaccttggatttccccctttggtttttggggaaaacacttcttggccattataaaattcttttcccaacgttgaaaaaaacccaatttttgttaccctaccgttaaccctctttgttacctccccggggggccgttaaaattcaatttcccggggccccattgggggcgggaaccttgaaatttcgggcccatttccccctatagtgattcttttaaaaattcacggccgtccttttaaaag

SEQ ID NO: 16 - DNA, 477 bp, unknown
  b1nr005, downregulated sequence from beech
acctcaaaccccatattccattacatctaaacaaacatcccatattccattatatctaaacaaacattcaccatttaaataatgatattgaagaaataaattatttcccagaaaaaagtgactctcagttaatggagtccaaggccaagatagtgctgagattcatggggttcatgtggtcagtagagccagacatgatctgctgaacaataattttgtttgccttttcagatggatggaatgcatcccaaaatgcatacaagtttcggttggggcacaagttggagagtggtgtgcagagtccaatgccattgaaaggtccttgtccacaacaagctatccttgatgtagtaaacccaaatgcttgagggtcagtaacgaaattcctgctcatttcttgtgtattagcagcaatgaagacatctctgccaatttcgttgttcagatttcttatcatttcaacaagctgtgggttgt

SEQ ID NO: 17 - DNA, 400 bp, unknown
  b1nr019, downregulated sequence from beech
ctttccagttaggccacaaaacactacaatgaagcagcaggttcaattcccatttcttcttttcttgatcttcttcctccattgcaccacaacttctgctcaatcccaagcaccatcaggccctacaaacatcacaggaatcctcaccaaggctggccaattcaccaccctcattcggttactaaaaagcacccaagaggctgaccaaatcaacacacaggttaacaactccaaccaaggcctaaccatctttgcaccaaccgacagtgctttctcaagcctcaaatcaggcgcactaaactctctcactgaccaacaaaaagtccagctggtccaattccatgtcctcccttctttctattctatctcacagttccaaactgtgagcaaccctttgcgc

SEQ ID NO: 18 - DNA, 713 bp, unknown
  b1nr025, downregulated sequence from beech
cttttttcagtctttgcttccatggcgttacctaagttgatcacagcacttctccttatattggttcttgcaagaatagagatttcaacaggccatgttgtgaagggcaagatctcttgccatgattgcactcacaatgatgtcctctcgggcatcaaggtttttgtgaagtgtgatagagttaaaaagtctgctacggcaatcacagaagatgatggctcttttgaatctgagcttccctcagaaactccaaaatcttcttctccactgaattgccatgccatgcttctcgggggtccaacacagctctatgccttaaaggaaaacttggtatccaaaattatcaaaacccacgagcctaactcctacaccatctccagtcctctcagcttttccacatcttgctcttctacaaaagaatctgcgaagtgcaaggacatcatgaacaagtttggttcttccaagactatcaatctgcctctgccaccggagtggggctttgcaccatctagcttctacatccctttctttcccatcataagcataccttgattatctatacactctctgagtaaataatatatatactggattggggtaaataaagggctaaaagggctgtgttttatgactgtgtgaagataccagaatggaactttaaaacttttaggaaatcctggatgggatgaaattttttttttccaagaaagatt

SEQ ID NO: 19 - DNA, 679 bp, unknown
  b1nr082, downregulated sequence from beech
accttgttcaatgttcttgggtcatccacagctctaatggtaacggttccaatgtcatcttccttgttaaaaattgccttgggatttccatctccaaagataatgactttttctctaggaggagcagtggctcctggctgtgacaatgtagggagaaacttgccagcgaagaagttggatgatacataagtgaagggaatgccttcagcctcaatggcacgtcggatttgggccttggtagcaaatgcagtcttggctggctcaacagcatggacacggtccacatcgattccgaattccgaagggaaaaatctcttaacattaccagcttctttaatggcagcaatgatcttgacctgatctactaactgaaggtgacccaccgttgatatcaccacatcaacatgcttaatcgcttttaccaaactcccatggtcgtaaagatctccatggagcaaagtgacacccaagttcttgaaattctcaatgagttttcccttaacgggatcagagactgtgctctctctcaccaaagcaaggtgggatggccagactttgggcttgcttcccgatgaatttccgatgtaggctgtgcctccaatgatcaaaatcttgcttttccaagcattttcatttttgggaggctaaaagggaaattcacaataacactagtggagaa

SEQ ID NO: 20 - DNA, 379 bp, unknown
  b2nr070, downregulated sequence from beech
tagcgtggtcgcggccgaggtgtgacggccatggatgtcgtttacgcgctcaagaggcagggaaggactctctatgggtttgggggttagggtttttaaatttctggggactttgtcaaaaagtcttgctgtcaagaaagtctgcaaatggtggtggtggtgtctgttttgtttgtaattagggtttgtaatatgaaatatgtgggtttctagggtttggtttaagggtctagttgtagtttttgttggctttgttttgtaattcaaactctgaattgcaatttcagaagatttaatgcaaagtaggttttgttaacttgaaattctctgttgaatgtcaaaattggaagccaaacgaatcagtgcattgtaaaaaaaa

SEQ ID NO: 21 - DNA, 348 bp, unknown
  b2nr074, downregulated sequence from beech
ttcgagcggccgcccgggcaggtacaggttggtttccataattccatttgatgagattttttgttttttttttttccactttactttaaattcattaataataactaaaccatacaaaattgggtgggggaatcatacaaaccaaaggttgattttaatgttgatttttggttactttgataactctgttttggttggcaattaaccctaataccattcagtttgaactttggcgcaccgaaaaagcaaccacctaaccccactgtctcaaccttgccttcatcgtctgaggattatcaccgtgggggcctaaaaggttttggaaacacctcggccgcaaccacccta

SEQ ID NO: 22 - DNA, 379 bp, unknown
  b3nr031, downregulated sequence from beech
tagcgtggtcgcggccgaggtgaaagagttgcggaggaagatcaattagctagtatagtaagggtgagtgaggtaaaggaacactgactgaaatggaagttaaaaatgcgaagggagggacatatcctctggcattgtttgtgatgctgatactcctctttaacgcatatcgttgtggtgctgctgtccttgttaagggcaacaccactgtccattgcaatggccgcctcgatgaatgcctgatcgaggacgaattggacttggtgttcctcaccaacccatacataagccggatacttgatgatgggaaacattataaatctgttgatgccgtcaagaaccgtaatcaaccttgtacctgcccgggcggccgctcgaa

SEQ ID NO: 23 - DNA, 478 bp, unknown
  b3nr056, downregulated sequence from beech
ttcgagcggccgcccgggcaggtacccgcgctgccacacacttggaacctcaatttgatagaaccacttggcgggctggccatgtcccacaccgccccataggccctacgcatggccctccattgctggcaatcctcccgccacaattcaacggctgtgatgtcgttttgtccagctgcatatataacgaataaagctaggtagtgaggaaatttgctgttctcatggaccttaaacatgaggttgtaaccagagtatgtgcatgagatccttcggtattctacatcaaccacaccgtaaggaaacaactccgaggccaagttgggacgtgccgaccttgtgaaggcacgtgggctgaggatgaagtcagttttgtctccctcaccgtagtcagtcaccactatgctcaccccttcatcaccgcaatattttggtttcgtgcacctaacctgatagcatacaccacacccagttccat

SEQ ID NO: 24 - DNA, 437 bp, unknown
  b3nr058, downregulated sequence from beech
ttcgagcggccgcccgggcaggtaccgtggtgatgttgtgcctaaggatgtcaatgctgccgttgctactatcaagaccaagcgcaccattcagtttgttgactggtgccccactggattcaagtgtggtattaactaccagcctcccactgttgttccaggaggtgaccttgccaaggtgcagagagctgtttgcatgatctcaaactctacaagtgtggctgaggtgttctcccgcattgaccacaagttcgacctcatgtatgccaagcgtgcctttgtgcactggtatgttggggagggaatggaggaaggagaattctctgaagctcgtgaggaccttgctgccctggagaaggattatgaggaggttggtgctgagggagatgagggtgaggatgatgaaggcgatgagtacctcggccgcgaccacgcta

SEQ ID NO: 25 - DNA, 263 bp, unknown
  b3nr083, downregulated sequence from beech
tagcgtggtcgcggccgaggtggggtctgctgatctgctccacaagtattaaccaacaaacaaacaaaccctgcgcttcctatagaattaactgacaaagaaattctcttcgtagatcggatcgtttttctgagtttcaaacaaaaatgtcttcagtgttcagcggcgatgaaacagccccattctttggcttcctcggcgctgcagccgccctagtcttctcatgtatgggagcggcgtacctgcccgggcggccgcctcga

SEQ ID NO: 26 - DNA, 410 bp, unknown
  b3nr095, downregulated sequence from beech
ttcgagcggccgcccgggcaggtaccctgaccggatgttgcttactttctctgtgttcccatcgccaaaggtctcagatacagttgttgagccttacaacgccaccctttctgttcaccagcttgttgagaatgctgatgagtgcatggtgttggataatgaggccttgtatgatatttgtttcaggactctcaagttaactaccccaagctttggtgatctgaaccatctgatctctgcaaccatgagtggtgttacatgctgtctcaggttccctggccagcttaactctgaccttcggaaacttgcggtgaacctcattccgttcccccgtctgcacttctttatggttggatttgctcctcttacctcacgtggttctcaacagtacctcggccgcgaccacgcta

SEQ ID NO: 27 - DNA, 213 bp, unknown
  b6nr003, downregulated sequence from beech
tagcgtggtcgcggccgaggtacttgagtgtgtctacggtgtgggtgttcaccaccgtcttctccaccctcttttttccttcctcgtcgtggctcacagctcggattgtcaccgtttcgttcttgtcgtcaatgggcttcattttcttcatcaaactgataacctgggttgaaaaatgcttcagataatgttgacctgcccgggcggccgcta

SEQ ID NO: 28 - DNA, 584 bp, unknown
  b3nr038, constant sequence from beech
ttcgagcggccgcccgggcaggtgagagagagagagagagagcgaaaaacagagagccgaagacaaagaagaaagagagggaagcgaaaagagatggcccgtactcttttggggtggtcatgttggaagttctttgtgggagacctgtcattgatccatcccttccaagggaaaaggtgaatctagttgactgggcaatggaatcacagaagagaaggcaattggaggaaatcgtagatcctcgtcttgcaggtcaaattaagccagattcgttaaggaagtttggcgacatagctgagaaatgcttggcagaatgtggtgttgatcgaccttcaatgggtgatgtgttatggaatttggagtttgcacttcaacttcaagggaatgaacaaagatccaattgtgatggcgagctgtcttcacaagttaatcatgtcaatcaagtggagagcagtgtatccattggacagttcagtatgggcgatgttgctggtatttcaatgagtaaggtctttgcccaaatggtgagagaggatatgaggtagctgatggtaaagcaatgtacctcggccgcgaccacgcta

SEQ ID NO: 29 - DNA, 209 bp, unknown
  b4nr068, constant sequence from beech
tcgagcggccgcccgggcaggtactgggaacataaaaaaaaaaaaaaaaaaaaaaaattttaaattcaaaaaattcgtttttaaaaaaaaaaaattttccaattgggttttacccatttttaaaaatttttcaaatttttttaaattgcacaattttaaatttcggaaaacccatttttttaaattaaaattttttttaaaaaaaaaaa

SEQ ID NO: 30 - DNA, 22 bp, unknown
  PCR primer b5nr019L
caggccaagggccaaactcagg

SEQ ID NO: 31 - DNA, 22 bp, unknown
  PCR primer b5nr019L
agcagccccttgtgccttagcc

SEQ ID NO: 32 - DNA, 22 bp, unknown
  PCR primer b5nr078L
agtgtgtgccacctgggactgc

SEQ ID NO: 33 - DNA, 22 bp, unknown
  PCR primer b5nr078R
ctaatctgggccgccaccattg

SEQ ID NO: 34 - DNA, 23 bp, unknown
  PCR primer b1nr013L
gatccaattgtgatggcgagctg

SEQ ID NO: 35 - DNA, 22 bp, unknown
  PCR primer b1nr013R
accagcaacatcgcccatactg

SEQ ID NO: 36 - DNA, 22 bp, unknown
  PCR primer b2nr070L
cggccatggatgtcgtttacgc

SEQ ID NO: 37 - DNA, 23 bp, unknown
  PCR primer b2nr070R
caccaccaccaccatttgcagac

SEQ ID NO: 38 - DNA, 22 bp, unknown
  PCR primer b3nr058L
tcccgcattgaccacaagttcg

SEQ ID NO: 39 - DNA, 22 bp, unknown
  PCR primer b3nr058R
tccagggcagcaaggtcctcac

SEQ ID NO: 40 - DNA, 23 bp, unknown
  PCR primer b3nr038L
gatccaattgtgatggcgagctg

SEQ ID NO: 41 - DNA, 22 bp, unknown
  PCR primer b3nr038R
accagcaacatcgcccatactg

SEQ ID NO: 42 - DNA, 631 bp, unknown
  SAM synthase 1, constant sequence from pear
gatgttggtcttgatgctgacaactgcaaggttctagtgaacattgagcagcagagtcctgacattgctcaaggtgtccatggtcatttctccaagaggcctgaggaaattggtgctggtgaccaaggccacatgtttgggtatgctactgatgagacccccgagctgatgccactcacccacgtccttgcaaccaagcttggtgccaagctgactgaggttcgcaagaacggcacttgtgcctggttgaggcctgatggcaagacccaagtcaccattgagtattttaatgacaagggtgccatggtcccaattcgcgtccacaccgtcctgatttcaacccagcacgatgagaccgtgacaaacgatgagattgccgctgatctcaaggtgcattgtgcaggtttcctatgccatcggtgtgcccgagcctttgtcagtgtttgttgattcttatggcaccggaaagatccccgaccaaggagatcctcaagattgtgaaggagacattcgatttcaggccaggcatgatctccatcaacctggacctcaagaggggtggcaatggaaggtttttgaagaccgccgcctacggacacttcggcagggacgaccccgacttcacctggga

SEQ ID NO: 43 - DNA, 472 bp, unknown
  ACO-2, upregulated sequence from pear
cttgttgtgtgagaaccttggactcgagaagggttatctgaagaaggttttctatggatccaagggtccgaattttgggactaaggtcagcaactaccctccatgccccaagccagacctgatcaagggactccgggcccacagcgacgccggtggcatcatcctgcttttccaggatgacaaggtcagcggcctccagctactcaaggatggtgattgggtggatgtccccccaatgcaccactccattgtcataaacttaggtgaccagattgaggtgatcaccaatgggaagtacaagagtgtgatgcaccgggtgatagctcagtcggacgggaccagaatgtcgatagcctcgttctacaacccaggcgatgatgcatttatcagcccagcaccggcagtgcttgagaagaaaactgaggacgccccaacttatcccaagtttgtgttcgatgactacatgaagcta

SEQ ID NO: 44 - DNA, 456 bp, unknown
  ACS3-4, upregulated sequence from pear
acctggggttcaaagggatcaaaaggagtgtctggctttagagaaaatgccttatttcaagactaccatggccttttgtctttcagaaaggcaatggcaagttttatggaacaaattagaggaggaagagccaaatttgaccctgctagggtagtcttaacggcgggtgcaactgcagcaaatgagctattgactttcgtcatagctgatcccggtgatgctttgcttgttccaaccccatattatccagggtttgatagagatttgaggtggaggactggtgtgaacattgtgccaattcactgtgaaagctcaaacaacttccagattactcctcaagctttagaagctgcatacaaagaggcagaagccaagaacatgagagtgagaggggtactattcacaaatccatcaaacccacttggtgcaacaatccaaaggacagtccttgaagag

SEQ ID NO: 45 - DNA, 418 bp, unknown
  PDC-6P, upregulated sequence from pear
gtatgagttccaaatgcaatatggatcaattggttggtcagttggagctactcttgggtatgctcaagctgttcctgagaagcgtgtgattgctttcatcggcgatgggagtttccaggtgactgttcaagatgtgtccaccatgatccgaaatgggcagaagaacatcatcttcctgataaacaacggcggatacacaattggggtggtgatccatgacggaccctacaatgtgatcaagaactggaactacactggactagttgatgccatccacaacggggagggcaagtgctggacaaccaaggtccgttgcgaagaggagctgattgaagcgattgagactgcaaccggggcgaagaaggatagcttgtgcttcattgaggtgatagcccacaaggatgataccagcaaagag

SEQ ID NO: 46 - DNA, 347 bp, unknown
  PC17-6, upregulated sequence from pear
gactgcgtaccagcttactttcttttgggtaattaggcctgacttggtcaacggtgaatccgcaattttaccacctgagtttgtggatgaaactaagggaagaagtctaatagcgagttggtgcccacaagagcaagtacttaaccacccatcgattggagggtttttaacacacagcggttggaattcaacaattgagagtgtgtctgcaggagtgcctctgttatgctgcccattccattctgatcagcaaacaaactgtcattatacttgcaaagaatggggcattggtatggagatggataagaatgtgaagagagaggaagtggagaagctggtacgcagtc

SEQ ID NO: 47 - DNA, 20 bp, unknown
  PCR primer SAM-1 (F)
tcgcaagaacggcacttgtg

SEQ ID NO: 48 - DNA, 20 bp, unknown
  PCR primer SAM-1 (Reverse)
cctgcacaatgcaccttgag

SEQ ID NO: 49 - DNA, 21 bp, unknown
  PCR primer ACO (forward)
gttgtgtgagaaccttggact

SEQ ID NO: 50 - DNA, 21 bp, unknown
  PCR primer ACO (reverse)
aaaagcaggatgatgccaccg

SEQ ID NO: 51 - DNA, 21 bp, unknown
  PCR primer ACS3 (forward)
tggggttcaaagggatcaaaa

SEQ ID NO: 52 - DNA, 19 bp, unknown
  PCR primer ACS3 (reverse)
agttgcacccgccgttaag

SEQ ID NO: 53 - DNA, 20 bp, unknown
  PCR primer PDC-6P (forward)
tgattgctttcatcggcgat

SEQ ID NO: 54 - DNA, 19 bp, unknown
  PCR primer PDC-6P (reverse)
ccgtcatggatcaccaccc

SEQ ID NO: 55 - DNA, 19 bp, unknown
  PCR primer PC17 (forward)
agcgagttggtgcccacaa

SEQ ID NO: 56 - DNA, 20 bp, unknown
  PCR primer PC17 (reverse)
tccatctccataccaatgcc

SEQ ID NO: 57 - DNA, 711 bp, unknown
  13nr042, upregulated sequence from apple
acaccatctttgngaccatcatcaatcattgcgagaccctcatctaataaagtgtcaaaggcatcgattgcacggccagtggtccaagatagccgtctctccacctcagggacggtcacaaagccttgagcctgggctagctctaaagtttcattatggtctttgntcaactcagttggaacagaacgaacaagctttctctttccaacagaaataacctcataaccattacctaataccttgagcttgcttatagcacgcaggcaaccatcctcggaaacagcttcacggtcactcttccgcctctgacgaagcagactgcagagttcctgcaggttgatcaaacccccattgnggggtcttgttgccaagcaaatgtcaacaatttgcaccccaagttcatagtagaaatcaccaatccccaaaagctctgcccagaaacccttattggatgccaagggatctactccgaccttcgtgcacatgttgtgaaattgtgatctgaaagctgggttctttcgaatgtcattcttgtgctttcgagcgaaatcttccaactgcgttcggaaagtggtgagctgctccttcatgagatcggttctgatcttggcaacattctcgcccagctgacgatattggtccctagcggcggcggtggtctgcaagccgccgattccagactgccccgggccggncnnntnaaatactnng

SEQ ID NO: 58 - DNA, 357 bp, unknown
  13nr062, upregulated sequence from apple
acctcagctnnctggtcaatgtcattaaagatgttgatgcacatcttatcaaggaataagcgccaagcacaagatttatgcatacatacgatatggaacattgagtaatattactatcttcaagaataaagacctgagagctgagcaagctagttagcactggtgcttatttcggtttctgatttggtttgtctttaattctatgagcaagaatgaattgagtgtgatattattataatgctgatgttgtaaacaagttcaatctattggcatgcttatattttttcgagaacaacttagttttttaactaaagtctcaaccttgtacctcggccgacctcggccgcgaccacgcta

SEQ ID NO: 59 - DNA, 249 bp, unknown
  15nr090, upregulated sequence from apple
acaaagcaaacagacaacaagcagccataacctaggaccagatcccagcctagtaggacgtgcagctcagaagaagccatggccactttattctaaaaacacgaccttcgtaaatttggatcatgattcatgattacgacacacacgcatacatatattannggtaaacatacaagccccgcgattctcttgcttcatcagaaggngacatctgtccctctcacttgcacctcggccgcgaccacgcta

SEQ ID NO: 60 - DNA, 252 bp, unknown
  5nr032, upregulated sequence from apple
acacaaagaggtngttcttgctgagccctacatcgccgtattgcatgacatgagaaccaaagccagaattgtcatttgctgtccttgttttaaccagttcatactgctggtgcagggtttctgacttcaagttgtgtatgtcactgtcttccatccacgcaacactatacaagtcacccaagcaggtttcatattctggaggaggactgggatactctccagggcagtacctggcccgggcggccgctcnaa

SEQ ID NO: 61 - DNA, 723 bp, unknown
  5nr041, upregulated sequence from apple
actcacgcttgtnctcgctaaacatgcgagcagcttctgaatttgcaggagagtttgggttgggatcacatagtagtgactggatggacgtgagtatagctgccacgtcatatataggactccactgattctgtaaaatatccaaacaaatacttccatccgcataantatttggatgaaacattctagaaacaaaccggactgttggtggtttgtttggataatcctccgtaaactgaagagtcaacttaaatgtgcctccatcccaaggtgtatcatcgggaccaaatataacagcattccacaacattatattgttatcctgtggcgccccactaatccctgcaggcgggtcctgttgcagcctcttgaagtctctcatcagcctcttccttgcaggagtcgacatcgctaatttgctaccacctggagctccgaatctccaagaggncccgatccgagcaggctccgaattgggttgctgaatcgggtgatcaaggaagggttttggaggtgggtttgatcagatcgaagccgaagccatacctgccccngggcgggnnnnnnaaatcacnnnngaattnnnggnnnnntgnngnnnnccnnnnngggnannctccnnnncnnnnngnnngnnnnnnntnnnnnnnnnnnnnnnnnnnnncnnnnnnnnntnnnnnnnnnnnnnnnnnnncngnnnnnnnnnnnnnnnnnnnnnnnnng

SEQ ID NO: 62 - DNA, 445 bp, unknown
  5nr069, upregukated sequence from apple
aataaaatggntgtgagtggagctggtacttgcaagctggagactatggaggctgaggtagagatcaaagccaacgctgataagctgtacaaattcatcagcaaccagcactatgacttccccaaagcagcctctgataaaatacacgatgttgcggtacatgaaggtgactgggaaacttctggctctgtcaaactctggaaatacaccatagatggaaatgtcgagacttacaaggaaaaggtggaaatagatgaagcaaacaagcgggtgagtcttacagggttggaaggatcacatgtgctggacaattacaaaagctacaagatcattttccaggtcactccaaatagtgaaggaggttctgtgaaaattactttagaatataaaaaactcaacgagaacgatccgcctccacagaagtacctcggccgcgaccacgcta

SEQ ID NO: 63 - DNA, 650 bp, unknown
  6nr013, upregulated sequence from apple
ggaaacgttatttcgaccgctgcaatgcgagtttttgctactctctacgagaaagacatctaattcgagcttgttccaatcgacatgagagcntggtgaacacaaaaaggagcccttcatatccctcaatccatttggttaagttccagctttcgaagacggagatcttaagctctttgaatcaagggcgattacacaatacattgcccacgagtatgctgacaagggaacccctctagtgatccgagactcaaagaagatggcaattatatcattgtggtcggaggtggaggcccaaaagttcgacccggcggccacaaaactgacctatgagctagctataaagcctatgtttaaaatgaccacagacgcggcagttgtggaggaaaatgaagccaagttggctgtggttcttgatgtatatgagactcgtctggctcagtcgaaatacttggcaggtgaaagcttcactttggctgatcttcaccacctccccaccatacattacttgatgggaacacaatcgaagaagctgttcgaatcccgcccccatgttctcgcatgggtggcggacatcacagcaaggccagcttggaacaaagccgttgcccagcaaaagtgaaacaagtacctcggccgcgaccacgcta

SEQ ID NO: 64 - DNA, 330 bp, unknown
  6nr096, upregulated sequence from apple
acgttagtaaccgtaacgcaatgacgaaaagaacgaagaggtaggattctcttccctactattacccctcacactcttctttgtcttattctgtatataaaaaatttgcagaggtttgttgcgtgcatttacagaaagtctttggcattgcctttgggattctcttgaaaccttgctatcctaaactgcttcagctccactccttcagcctccccagtaagcgcaaggtcagccaaaatcctccccaccaccggcgacatcttgaacccgtgacccgaaaaccccccgcccaccaccacatccttccacctgcccgggcggccgctcgaa

SEQ ID NO: 65 - DNA, 447 bp, unknown
  7nr006, upregulated sequence from apple
ctgatacttacacagtcggagacgatttggaatggactattcctccagccggctctattgcctacaccacctgggctaacacaaaaagatttcagaataatgatacaatagtattcaagtggagtggatcacacacagtagctgaagtatcgaaggctgattacgataactgctcaaattcgaaccatcttgctttttatgattccagccctgcaagtattacactgacttccaacgtgacccgttacttcatctgcaccgtaggtaatcactgcagtgatttaggacagaaggtgactatcaagatttctgaagatgatgatcgctggtgggaccgcaacgcctcttcttctctcaccgttaatattggtgccctaatcctctccactgcgatggccatcctcttctatagctttaattaagtgtacctcggccgcgaccacgcta

SEQ ID NO: 66 - DNA, 435 bp, unknown
  8nr019, upregulated sequence from apple
actccatggagatcagttctgttgcttatgaccagctttggcgctttgacatggaagctttgccagcagatctcatcagaaggggaatggcagttgaggatcctacagctgagcatggcttgaagctaacaattgaagactacccatttgcaaatgatggtctcatgctgtgggatgccattaaggagtgggtgagtgattatgtgaaccattactatccagacccaaatcttattgagtctgatacggagcttcaaggttggtggacagatgttagaaccaaaggtcatgcagacaagaaagatgagccatggtggcctgttttgaaaaccacggaaagtctgattcacgctttgactactatcatttgggtaaccgctggtcatcatgcagcagtgaactttggtcagtacctgccccgggcggncgctcnaa

SEQ ID NO: 67 - DNA, 742 bp, unknown
  11nr011, downregulated sequence from apple
cggcacgagggtcccctcccccaccatccccttcaccgcctccccctgtagtccaataccaatcaccaccaccgccaacacctgttcactacaattccccaccaccaccatccccatcacccccaattccatgtgaaaccccgccacctcatccatcaccacccccaccagttcaccatctttcgccaccggttcactacagttctccaccaccaccaattgtttatgaaagcccgccgcctcccactcctgtatatgagggcccattgccacccatctttggagtttcgtacgcgtctcctccgccgccacccttctattgattcttttgagaattttctcctcctctaacctttcccagagcacaatcaccaaaaactcaacagtcctcgttcacattcacggcttcttctntcccccacaccatcatttctctctctttcgggcaattctcatcgctcgtgcagagttagaaaagcaaaaaaaaaaaaaaaaanntnggggggggnncgggnnccaaatccgnccnnntggcggngnccntatnncantnnnnngncnnngnttcnagaaaanatnnagcnganggtntgnnnnnntnngcnnaanaannnncangtnnannnancccngtantntcncaannntnntatntcnanagnntnnttgatntttctttcctcaattnncaatangnnngacnaanaantncatacatnaannnatnnnacgnnnggagcnt

SEQ ID NO: 68 - DNA, 406 bp, unknown
  12nr048, downregulated sequence from apple
acaggtcatgtgaagacctagttgtagctatttccaagagtatccaatagtttttagtcaacaacttcgtagattcattagccaaaaatgcatctctctcagcaggagccagagtccacagcaccacagccctctcgaaatcacttgagagttctttgtccaacgacttgagaagatcttccccataggtttcggcgtatgtctgctgaatcaattttctctgagctgcattcctgtgagtcagaatggatatgatcaatccctcatttgtgccccatcctgcaaaagctttcctgagctgctcagcgtcttccactggagaaggaaccacatctggcaccctgtaactcgccattctttctctctgtctgtttttgtatgtaagacctcggccgcgaccacgcta

SEQ ID NO: 69 - DNA, 695 bp, unknown
  12nr056, downregulated sequence from apple
acgagaagtatcatgtctgttatggaggagatgaggaagctagaaagtccaactacactgacatggttaataaatactatgatttggttaccagcttttatgagtatggcctggggagagtctttccactttgcacccagatggaatggggagtctcttcgagagagtatcaagcgacatgaacacttccttgctttacaactaggactcaaacctgggcagaaagttttggatgtaggttgtggaattggtggaccactgagagaaatatctcgcttcagctcaacatcagttactgggttgaacaacaacgaatatcagatcacgagaggaaaggaactaaaccgtattgcaggagtggacaaaacctgcaactttgttaaggctgacttcatgaaactaccatttcctgaaaattcatttgatgcagtatatgcaattgaagcgacctgccatgcaccagatgcatatgggtgctacaaggagatttacagagtattaaaacccggtcaatgttttgctgcatatgagtggtgcatgactggtgcatttgatcccaataaccaagaacatcaaaaaatcaaggcagaaattgagattggtgatggcctcccggacatcagattgacgggaaagtgtcttgaagctttgaaacaagcaggttttgaggtcgtatgggagaaagatcttgcggg

SEQ ID NO: 70 - DNA, 345 bp, unknown
  12nr062, downregulated sequence from apple
acaagaacaatgaagccagaggaatcccatacccaaagctccaaccaatgggggtgttttcgacattgtgggaggccggcgattgggctaccagaggaggacttgagaagatcgactggagcaaagtccctttctattcatattacaaagattttgacattgagggatgccctgtgccaggaccagcctcatgtgcctcaagcaccaacaactggtgggaaggcgcttcataccaagccctcaacgcccttgagtatcgaagatacaggtgggttcgtatcaaccacatgatctacgattactgcaccgacagatcccggtacctgccccgggcggccgctcgaa

SEQ ID NO: 71 - DNA, 714 bp, unknown
  12nr094, downregulated sequence from apple
acgatacttcctgtcgatactgtagatgagctgcatgtcttcatcgctcagctggaattcaagaacctccaaattctctttcaatcgctgaattttcgatgattttggaatcacctgctgttttcctctgaattccccacctcagacagatttgtgccacgctctttccgtatttcttagccacatcattgagaactggatcatccaaaggtgaaacagaaccaaacatatccttgttggcagcagcacctccgagaggggtgtgagctgtgggaagaacgccgtgtttcatacagaatttgacgagagagtcgcgctggaaataggggtgggtttcaaattggctcacagcaggctttattttggagtaagccaagcaatctctagttagaaagagctcatagttgctgagaccaatgctgcgaactaagcccaaagagacggtcttttccatgccctcccaggtttgttgaagggaaattgttacatcgatgtccaacaccttatcctcgcccaaaagactggcagttttaccaattgcattgtgctttgtgggcattgggtagtgaaccaggtagagatccagataatctatctgaagcttctcgaggctgttcttacaggcctccaccacatgcccatggtctgaattccaaatcttggtggtaatgaaaagttcttccctcttaacaagtccagtcttaaatgcttctg

SEQ ID NO: 72 - DNA, 357 bp, unknown
  1nr054, downregulated sequence from apple
gagagagccatggatccaaaccagcagagaccaggaattccaccaaatcgccctcagccaccacctcaaggcggcggcggcgactcctcggccattctctttgtcctcttcgctttcctcgccatttttttcgccgtgaccgttcttccaacatcatcaactatatcaattctgcaccaagtcccagaaggtcatgttggggtatattggagaggaggcgctcttctgaagacaattacagatccaggtttccatctcaagctacctctggtcacccattttgaacccgttcaagtgaccctgcagactgatcaggtaagggatattccatgtggtacctcggccgcgaccacgcta

SEQ ID NO: 73 - DNA, 249 bp, unknown
  1nr089, downregulated sequence from apple
gttgaaatcggccctaaaaagttgaatgaataaccccattgtgtttaaagttagttgctgtattcaaaaggaattctaagcatcaatttcattaatttgcttatcatgcttttaaaaaacgtttgtttccttacctttcattgttagccaatcacccctaaccccgttacaacccttaacttcaatcttgagtgttatgtgtttcaatatgtggagtttgggattggtacctcggccgcgaccacgcta

SEQ ID NO: 74 - DNA, 327 bp, unknown
  2nr039, downregulated sequence from apple
acttgtttgggcgtgtgagcatgaagatcaagctcatccctggagactctgctggaaccgtcaccgctttttatatgaactccgacaccaatacagttcgtgacgagttagattttgaattcttggggaaccggaccggacagccttatacagtccaaaccaatatctatgctcatgggaagggcaatagggagcaaagggttaatctctggtttgaccctgctgcagacttccacacttacactatcctctggaaccatcaccatattgtcttctatgtggatgatgtgcctataagggtgtacctgccccgggcggccgctcgaa

SEQ ID NO: 75 - DNA, 182 bp, unknown
  2nr080, downregulated sequence from apple
accccagatcactgtctgcttcgacattgacgctaacggtatcttgaatgtctccgccgaggacaagaccaccggacaaaagaacaagatcacaatcaccaacgacaagggcaggttgtccaaggaggagattgagaagatggttcaggaggccgagaagtacctcggccgcgaccacgcta

SEQ ID NO: 76 - DNA, 408 bp, unknown
  3nr034, downregulated sequence from apple
acctttgatccgtcgagcgagagcccttccacacgggactcccggatcactatggccgactttcgtctctgttcgaccagtaggtctcacagtcaggcaggcttataccattacgctcaacagcagaatcttagcttgagcctaccttcgcacacctccgttactctttaggaggcatccgccccagataaactacccacctcgcagtgtcccgccccccccgaattatcggtgcggcggttaggcatccttagacgaaagagtggtctttcaggattggtcgttgtgtgtcaccacctcccacctatcctacacattcgatcaaggttgtcactgcgaagctatagtaaaggtgcacggggtcttaccgtctagccgttggtacctgcccgggcggcccgctccgaa

SEQ ID NO: 77 - DNA, 661 bp, unknown
  OProseR1069 from rose
aaatgaagagagcgcattctccaagatggtcaggagtacaggcccttctaatgcgcagtacttacgcagcctggtatttgaaggcaagcagaataaggtgaatagagacgaagctgagcaactgtttggccagagccagaggagatggttagcctcttcgcgctgggcagctgctgcccaatttgcattggctgtcagtctcacatcctcacagaacgatctccagaggttagatattggagacgaggacaatatcctcatgaaaacaaaggatgcagttataacactacagggagttttggaaggaaagcatgatgaagatatagacatatcactaaatcagcaccatattccaagggaggggtggtggtcagctttcttcagaatagttgaaggtctggccgtgatgggcaggctggctcagaacagactccaccctttagaagatgacgattttgaagaccagtctttatactagtaatttcctctgaatggagaaggctatggattcaagatgttatattgtatagattagagtatagctaatctggatgatatccaaattgcaacatgcagaanagagttacatctgcatagaacaccagaacaacaatcatagcagtagttattaattttaatttatgcttaaaaaaaaaaaaaa

SEQ ID NO: 78 - DNA, 670 bp, unknown
  OProseR1072 from rose
cttaaaccaaactttcgttacgcgcgctattcagtgtaacactttagggttctcgacatgcacatccgtcctgctttccatggggctctactgggtctctggatctgcttgtttacttcaagcttctgctacggtgatgagaagaccatcgaggcagccggagggccaagcaaagttttctcagagttgaaaacacctgaggctgcagagctcaatgaagagggaaagtttgcagtactaccaccaaagccgttcttcaagaaaccacttctgcacaaggttcccactctgaagaagccatttccacaaaagccgtacttcaaaaaaccacctttacataatattcccattgtcaaaaagccatcaccaccatctgttcctgttttcaagatcccaccgttcaagaagccgattccttccccaattccacaagaagaaaaatcttttttgaagaagccatatttccatccagttccagtttacaagaagccaattttaccccccgtgccggtttacaagaagccatatttccatccagtgccggtttacaggaagccaattttccccccagtgcgtgtttacaagaagccatatttccaccccgttccggtttaaaagagacgcatgttcccccccggggcctgtttagagaaagccctgtcgccctccg

SEQ ID NO: 79 - DNA, 436 bp, unknown
  OProseR1093 from rose
aaaacccccacccgagtgtgtcgccggcctctaaattgcagccagttacctattatcaacctattcatactgattctgggtgaagaggtccagaatattacatgttgtcatacaaggccaaaaatttccgatgtgggcctcttctatgttctgaacataaacgtatgttcgttggcgtgaattaaaaagtcgatgcttaagcgacgtaaggcgtacttttactctagaaagcaataagagccatgcatagtaaaagggtgtataggggaatatggaaattgttgttgtgtgtatatgtggatgaagaaggcccaatgaggcaataagtctgttttacaagtgagcaaatgtcaattcctacttgtgtctgtatccagctcccattattgtaccaataaagctctcctgcgctattgtgaaaaaaaaaaaaaaaaaa

SEQ ID NO: 80 - DNA, 666 bp, unknown
  OProse1094 from rose
caaaacaatcagtagagatccaagatccagacgacgtcgttgcagatccactccccacgatgtcttctaccttcagcggcgatgagacggcgcccttcttcggcttcctcggcgccgctgcggccctcgttttctcctgtatgggagctgcctatggcacagcaaagagtggggtaggtgttgcatctatgggagtcatgaggcctgagctggtcatgaaatccattgttcctgttgttatggctggagttttgggtatttatgggcttattattgctgttatcatcagcaccgggattaatcccaaggcaaagtcatattacctctttgatggttatgcacatctctcctctggtcttgcttgtggtcttgcggggctctccgctggaatggcaattggtattgttggtgatgctggtgtgagagctaatgcacagcagccaaaactctttgtcgggatgatcctcattctgatttttgctgaagcattggcactgtatggcctcattgttggaatcatcttgtcctcccgagctggccaatccagagcagactaagaagtggctgtgcagtctaatatactttaattggcatatgcacttagctggggaattttttggctgctctcaggttgctgattggttatgcttgcagattcctaaagat

SEQ ID NO: 81 - DNA, 674 bp, unknown
  OProse1100 from rose
attcagttaatctcttaggaaatctagttatcaatggagtctctggctaaacaggcaaaggttccagccacaatactagccattgggacagcaaatccaataagctgttattaccaagaagactatcccgatttcttgttcaaagtcaccaaaagcgagcacaagaccgaattaaaagacaagttcaaacgcatatgtgaaaagtcgatggtaaagaagcgatatctgggcgtcacagaagagagtctaaaagccaaccctaatatatgcagctacaaggctccctcactcgacgcacgtcaagacttactgattcacgaggtccccaaactcggtaaagaagcggcgttgaaggccatcagagaatggggccaacccatttcaagcctcacccacctcatcttctgcacagcttcctgcgtcgacatgcccggtgccgactttcagctcatcaagctcctcggcctaaatccatctatcaaccggttcatgatctaccagcaaggctgctttgctggtgggaaggtgctacggattgctaaagatgtggccgagaacaatgccggagctcgtgttctgatcgtgtgctgtgagatcaccaccatgttttttcagcaaccttgtgacagccacttggatgttttggtcggacaggctctgttttcagacggtgc

SEQ ID NO: 82 - DNA, 677 bp, unknown
  OProseR1117 from rose
caggaacagagccatatttggtctcacactaccagcttctagctcacgcagcagccgtaaagttatacaaggagaagtatcaggctgatcagaagggtgtgatagggataacaatactgtcacactggtttgtgcccttttctgatgccaagcaccatgaagaagctgcgtgttatccgtcacactggtttgtgcccttttctgatgccaagcaccatgaagaagctgctctacgagcattggattttatgtttggatggtatatggatcccttaacaaacggtgaatatccgcacagcatgagatctcttgtcggagatcgattacccaagttcactaaagagcaatccgagatgttaaaaggttcatttgattttctgggattgaattactacaccgctaactatgctacttatgcacctcatctcaagaatgctgcaaaccctagctactttacagatgctgtcgctactgtttccaccgagcgtaatggtattcctattggtcaaaaggctgcttcagattggctgtatgtttatccagaaggatttcgagaactattgctttacacgaagganaagtancataatccacttatttacatcactgaaaatggaagggatgagcacaatgatccgaaattatcacttgaggaagccctagccgatactcaccga

SEQ ID NO: 83 - DNA, 674 bp, unknown
  OProseR1198 from rose
attcaactaaactagtatgtaaaatgccatcccggctaattaactggcaggtgcaattttttgggagggaatgggacttcatggatttataccatattactctctttctgggagtccctttcctctgtcttttagcaccatttcaattcacttggggtgcactttgggtggcaatatcactatatttggtgtcgggtatgggtgtaactatctcttaccatcggaaccttgcccaccagagctttaaggtccccaaatggcttgaatactcactggcttattgtgcagttctgtcacttcagggtagtccacttgaatgggtgagtagccatagataccaccatcaatttacagacaaattgagagaccctcatagccccactaagggattttggtttagtcacgtgaattgggcgttcgattatcattctcggtttggaagctatgacggacaactgatgaagaacgtgggagatttggaatgccaactatactataggtttcttcattatacctacttccttcattcagttcttcttggagttgcactctatgtggccggaggattaccttttgtggtttggggaatgggtgtaagggtggtagtcgtttcacaaattactttttcaataaattctatttgccacacttggggaaaacaatatgggatactg

SEQ ID NO: 84 - DNA, 678 bp, unknown
  OProseR1208 from rose
cttataagcttcacccaaagaaaccgaagtgggaggaatatggctcggcattcaggagtcctagtagtattggtggtggtgatgatggtgagctggagcaattgcatgatgagcgacaacaacattgtttctaccactgctggtaatagtactgcggtcgcctcttcagcctggtgtaacgggcacataatcgacggccagtgcgtacttgttgcagacgcggcttacagtggcggcgagcacttggacttcaacctggacgtgatggacatggtggcgatggagtactataattcaggtggaagtaacagaagggtgttactgactggtggtatagacattaaaaaagctctaggtgctcaaaagcctgtggttgatgaaaaaaacaggaaatatatcaacaatagatgtgccacgtacaatcgcgtctgtcctaattaaatgatattgcaaccgatcaagtagcgatgatcgatctccaattttaatttttatcaaaactgtccgcaattcttaatgtattattaatcatatgtgtatatgggtttaagttactaaataatgtctatgggaggtgggaagcttggacagaccatttgagttgtggactacgtcactccctcttgcttattccactttactatttctctaagatttatgtatgtattnaccatcgtc

SEQ ID NO: 85 - DNA, 666 bp, unknown
  OProseR1246 from rose
gtttagccctccgtctatttcatcaccgtgtgtgtttctctgaaattccctgcttctactaccagaggtctgagaaccaatcatgtcgtcgtcttcttctatctcatcctcgtttcggttattcactaccaaaatcccccactcttcttctctaaaccccaaaaccctctccgccttcccaaaacacccgaacttctcttccttccgtcgacccatttctttcgaaagaatccccaccatatcctcatcccgatccccatctgccttagcaccatcagcttcaatgcccctgcaacccatcgaagaactccccccgaagcttcgagaaattgtcaagctcttccagtccgtaccggttccaaaagccaagtacgaggagctcctgtactacggcaagaagctcaaacccctcgaagataagtacaagacaaaagagaacaaggtcgaaggttgtgtctcgcaggtttgggtgcgagcctacctcgattccgacaagaacgtgttctttgaggccgattccgacgcgcttatatcgaaaggtctcgctgctctngctcgttaagggctctcgggtgaacccgtggaggaaattttgagggtccgacccgatttcgcgctccatcttgggcttcagcagagcttgtcacctggtaggaataatgggtt

SEQ ID NO: 86 - DNA, 527 bp, unknown
  OProseR1322 from rose
tagcgtggtcgcggccgaggtaccagcagcataacgcaactgtccatcaattgtggtgccagatgtatgaggggtcatggcatggtttggcatgtaacgccatggatggtctttgggagctggttgggggttccaaacatcaccactgtatcctgctatgtgtccactgttgcaggcatcgacaactgcttgcgtgtccatgattgccccacgggcattgttcacgatcaggactcccttcttgagcttggaaattctctctttatcaaacatcccccctgtcttttctgttagtggggtgttgacaacaacaacatcacattttggaagcatcttatcaagatcctcttcaaacttagcccccgtctctttttcaaattcggcatcagccttaattctgtacctgcccgggcggccgctcgaaatcgaattcccgcggccgccatggcggccgggagcatgcgacgtcgggcccaattcgccctatagtgagtcgtattacaattcactggccgtcttttttacaa

SEQ ID NO: 87 - DNA, 517 bp, unknown
  OProseR1391 from rose
ttcgagcggccgcccgggcaggtacttcgatacaatgaaagagatcggtgcatcttcaaagtccaattcagttttcatcccacatggacctggtgctgtgaaagacattgcttcacagataagagatggtctccttcaaggaaattcaactcaccagtaaagtcctaatacgaactatgaagttggaggtgcggcgtctataagcttcaacggttacttgttagctctatcgtgcaaggcaactaagaatcaatttgggtgtgctttcgtcatttgacatacatggtatatagtaggtggatttgagcgtgtttatatgcttgttcatagttggttgtctatattttattcagttcgaagcgatgtgaactatggctggtttagttttttcctgagcagcagggtttctcttttgaaaaccatacctgaataagcctagaattcgttcagataggccaagtattatgtagtaatgccaaggtgattatttggttattaatttttcaattttgtgaaa

SEQ ID NO: 88 - DNA, 689 bp, unknown
  OProse1459 from rose
ttcgagcggccgcccgggcaggtacatttgaaactgcagcaaccattataattcccagtaacaaacctcaactttgactctatccccaggcaaaatcctaatgaaactctttcggattttcccagaaatgtatcctatgatgttgtccgcgttgtctaatcggattcgaaacatgccgttcgggagcgactccgtgaccagaccctctgcgctgaacttctgctcctcaactttcggggatttactcatggcaacgatgttgggacaacgtttggagacgaggattgaggttccgaggtgaagctgaggttttgggtttggttggaatttgagggagggtggggtgagggttggggttttggagtggaggattgggtggtggagtgaggatgatgatgttgaggttaacatggtggtggttaatggtgatggggtgatgagttctggtgctctctgtgttttatgggtggagactgacagaggtggaccggaatagacttcaacgacttctatcgcgaatccacgtgggatagtcgtggtgaaaaccttatgggatgactctgggtgtttcctcagcaggtttgtagatctgaaagtcctcgggcatagagctttgcgaggggggattatagccctccacccacttcttattatcttccagctcgctgcgtgcctaccatcggtgggga

SEQ ID NO: 89 - DNA, 532 bp, unknown
  OProse1481 from rose
tagcgtggtcgcggccgaggtacgttgagagttgagtgagagaatacgacgagtttcagaaatatgttgagagtaggtttcttaatgttgcttgccatggctttggcagcaactagtgctcatgctcgcttagatctatttggccagctgctcacctccaagaaccaaccaaatcccaattcagaatgccagaaaattccatttctactatgcgaccgtgtctgtgacgtttgcgtttgcgatagacgtgctccagagtttgcagagtgcttttgcggacagtggaaaccttatgcagaacttcaaaaatctgacgaagcacaagtgcaggtgataggattagaaaaatcttctattaaagccaagtcttcgggcgccattccatatacagaatgcatcaaagtctgcaaaatttgtgcttgtacctgcccgggcggccgaaatcgaattcccgcggccgccatggcggccgggagcatgcgacgtcgggcccaattcgccctatagtgagtcgtattacaattcactggcg

SEQ ID NO: 90 - DNA, 485 bp, unknown
  OProseR1674 from rose
ttcgagcggccgcccgggcaggtacgaggagctcatcgaggaggcgcgtgacgagctcacgctcatcggaaagatgatcgagtgggatccatggggtgttcctgatgattacgagtgtgaggtgatcgagaatgatgctccagtccccaaacacgtgcctattcaccgacctggtcctcttcctgaggagttttacaaaacgctgcggggtcttggttcagaccaaacaaagttggatgaggcaaaggccacctcaattgaatctgaatcaaaggcataagggtctgctgtttctgttttgaagaactatagattccaaatttgaatttttatctttctcttggaactctcctgcttatctttgattacaatgggtttgtttgtagatttaaattatgggagagagaggcttcaatatttaataatctgcacttgtggtgtttgtagtaacaatgttgtattcccaaaaaaaaaaaaaaaaaaaa

SEQ ID NO: 91 - DNA, 476 bp, unknown
  OProseR1700 from rose
ttcgagcggccgcccgggcaggtacaactttatttgacaaacaagtgcttcatagttcatactaataatactcatgcagctagcacgcctgacattaggcgaggcagcaaacggtatctaattctcagtttcatgagcaccaggtccatgccctccgtgccctccacggcctccatggccaccatgtcctccgtgtcctccatgtcctccatgtccgtgtcctccttcgcctccatgtccatggccaccatgtccatgatcgttgccgtggaagtggtcagaattttcatgagctgagacctcagaggagatgaggagaacagcagagacaagggcaacgagaagaatagtcacctcggccgcgaccacgctaatcgaattcccgcggccgccatggcggccgggagcatgcgacgtcgggcccaattcgccctatagtgagtcgtattacaattcactggccgtcgatttacaag

SEQ ID NO: 92 - DNA, 636 bp, unknown
  OProse1727 from rose
ttcgagcggccgcccgggcaggtacctcctcctactgtgccgacctcaatagaaggcatggtcactgagacgtgaagatccttggcatcattgatggcttccatcatggtgatgcagtgagaactctcgatgttctgagctggatcttgaccagtggctaggtagactgctgaaacaatgttactggcatgtgcattgaaaccaccaagggcaccagcaattgcagaaccagttaggttcttgagcatgttaagctccaccaaggccgccacattggttttcaacaccttcgtcactatgtctcccttgataacagcctcgcaaaccacagatttgccacggccttcaatccagttgacagcagcaggctttttgtctgagcagtagttaccagaaatgccaatgacatccatgtcagggaagtcattctggaggaaatcgagaacgttttgaacacctttatagaccatgttcatccccatagcatcaccagtgctgcagcagaatctcatgtacctcggccgcgagcacgctaatcgaattcccgcggcccgcatggggggcgggagcatgcgaacgtcggccccattccgcgtatattgagtcggattacaattcaccggcggagggaatgaa

SEQ ID NO: 93 - DNA, 565 bp, unknown
  OProseR1783 from rose
ttcgagcggccgcccgggcaggtacgtcctgttgaggctgatggagtagtggtgaacaacaatgcacaacatgcttcactccttgataaaatggactgcggtggagcgtgcactgcgaggtgtcgtttatcatctcggcctcggctgtgtaagagggcatgcgggacttgctgccagcgttgtagctgcgtccctcccggcacggctggaaactacgacgtctgcccctgttacgctagcctcaccacccacggcggcagacgcaagtgtccttagttgaacaacattaaaactacttcaagcaagcttagctatctagctagctagcttggttatatatgtaccggagctactgagtgaataaaaatgcatatataggtttaacttatccaataaaaatggctactgcttgtagccttgtaatttctgcttcatattgtacctcggccgcgaccacgctaatcgaattcccgcggccgccatggcggccgggagcatgcgacgtcgggcccaattcgccctatagtgagtcgtattacaattcactggccgtcgtttttggcaa

SEQ ID NO: 94 - DNA, 684 bp, unknown
  OProseR1792 from rose
tagcgtggtcgcggccgaggtacaacaaattacaaagagatgaggttgtggcagaatggaggaaagtaaaggacaagatgtctctccatgttcattgccatataagtggaggtcatttccttttggatttgttttcaaggctgagatacttcatcttctgcaaagagcttcctgttgttttgaaggcctttattcatggagatggcaacctgttcaacagttacccagaattggaggaggctttggtttgggtttactttcactccaacattccagaattcaacaaggttgaatgttggggcccactcaggaatgcaacagcaccgtctggtaggggacaccagaaggcagcttcctcaagcaaccaggaggactttgtgccagaaccatgccaagaggactgtagctgttgcttcccaccactgagctccatcccatggccccaagagcttccccagccagatgaaactggatatggggcccagcagagctttctggggaagacccaagaaccaaactaaaatttttctctggttcttcatattggttaatgtaagaacaaaaatgtaaatagagacctagttatttgtcccattagggttttcatagtctttgtttggacttaggttgatgcatatgtatatacctgtccggtgactccagtccgattccagattcttctgg

SEQ ID NO: 95 - DNA, 526 bp, unknown
  OProseR1807 from rose
ttcgagcggccgcccgggcaggtacactgatgaaattgagcttgaagatgctgtgcacacggctattttgactctgaaggagggatttgaaggacagatctcagggaagaacattgagattggaataattggcgaagacaaaatattcagggtgctatccccagcagagattgaagattatttggctgaagtcgagtagtgttcctgttaccagacacaatctgcaatggttataggttgcaagatggttgatagtgcgaaagtgatttaatgtgttacaattatgtcgatcttttggaaccaaatcacaaaccttttgggctgaagcagcattttgccatcattaatagccctggatttttaagccttcaaactcttaaagactcattccgaacaatttgtacctcggccgcgaccacgctaatcgaattcccgcggccgccatggcggccgggagcatgcgacgtcgggcccaattcgccctatagtgagtcgtattacaattcactggcggtcgtttttacaa

SEQ ID NO: 96 - DNA, 694 bp, unknown
  OProseR0008 from rose
ctccctcccttcccccctactaactaagatggagagccagtacataggaggcaaaagcaataccgatggcggtgggatgccggagaaggaagtgagggtagcaaaccagagaagagttgggagttgtgagtcacttgtgagggttttggccgtattactaactctggccgctgctatagttcaagggttgaataagcagaccaaagttgctccaattaaggtggttccaaccttgccagccatttatattccactcacagccaaatggcaagacttatctgcttttgcgtacttgctggttgcaaatgtcatagcatgtgtatacgcagcattttccctagtcctttcctttgcaaaccggggcacaaagaagagctcactagggctattcatcattgtccttgatatattgacagtgggggtgcttttctcagccatcggagccgccatggctatcggtctgatcgggtaccatgggaattcacatgtgcagtggaacaaggtctgcaatgtgtttgacagattttgtcaccaggtggtcgcatcagtggtcctgactcagtttgcagctcttgcatttatcttcctagttgtgcttgctatcttacgccttaacagatcgaggtccacttaagtaggcaaacctagcttacatggactatatatatcgtttgtgaaccatagtagagatgaa

SEQ ID NO: 97 - DNA, 580 bp, unknown
  OProseR0053 from rose
ttcggcacgaggagagtgttcaaaaatgctgaatcactgggtgttccattcccaaagaaccaagccatgaggatttactcgagcctttggaatgctgatgattgggctaccagaggaggattggtgaaaactgattggtcaaaggcaccctttacagcatactacagaaacttcaacgtcatcgatgctaaatcatccaaatcattctctgattctcagcctagttggcagaccaatgcacttgatgctcctagccgaagacgcctgagatgggttcagaagtacttcatgatctacaactactgtaccgatttaaaacgcttcccacgaggttttcctgctgagtgtaggaactgatctttccagctaatcgttcagtttgaatgcattctttagtactgtctgtgtatacagggttggtgttaaagccatgtcaaataatgagttctttgtagctttggtggggggagtgatggtcaaaccaacaccaaatttgtaaactagcatttgttgaatatctgtttcatataataaaatttcagtgtccgttgtttagtttctcaaaaaaaaaaaaaaaaaa

SEQ ID NO: 98 - DNA, 726 bp, unknown
  OProseR0060 from rose
ttcggcacgagggattggtaaacataagcttcagattggagatctggagcatcacatctccattgaagccaatttaggagatattatatcttaatcaggaactatcaaaccaaaagaaaagaaatttgatagtcacccattattatatcatagtattaattagttcctagtagtcttccttttcccatatcaaggtggaaaagtccactatactggtgaaggaaacacgacacaagatcatagtgttcagcaaggtttgaaataaatactcattttgagttattccctcgtgccgaattcggcacgaggtgaagctaaaggaatcccatacccaaagctccaacccatgggggtgttctcaacattgtgggaagctgatgattgggcaacaagaggagggcttgagaagataaactggagcaaagcccccttctatgcttattacaaggactttgacatcgaaggatgctctgtgccaggaccagctaattgtgcctcgagcactaataactggtgggaaggcactgcttaccaagcactcaatgcccttgaatatagaagatacaagtggggtacgtataaaccacatgatctacgattactgctccgacagatcccggtacccagaacccccaccgtattgtatcgccggtctctaaattgcagccaggttacctattatccaccctattcatactggattctggggtgaagacgtccagaata

SEQ ID NO: 99 - DNA, 684 bp, unknown
  OProse0106 from rose
aacaaatggtcaggtttcatttatcccattcggatggggtcctcggatatgcataggacaaaactttggttggatggaagctaaaatggcgtagtccttcatattacgtagcttcacatttgagctttccccatcctatactcatggtccaatcacagctataacgattcaaccacaatatggtgcacacattatcttgcaaaaactgtaactgcagtgcggtgtaaaatgaacttcattttgttgcaaccactaaggaagttgcatgtataaaccgttacatagtgatggtttcatatgtcgtcactggaatattagtagaaattgttatatgttagttgttgctcaagatatcgaacaatgagggtgttggtgccttctaggggggtgccggacccctattgtccaagcaagttccatataccatgaccaagtgcgacgttttcgaaaaggtcgttgaggctatctacaccaatgtcgataaatccaagacttccgatggaatgtcaaccaccatacacttgggatccggtcttatcaccggtatggctgatgccgtagtctcacagccaggtgataccgtgttgtcaaacattcacaataccaagggtcttccaggacagggaaccacttcccgatggatcaatatccgtagagactaggcgttaagtaggttcctacact

SEQ ID NO: 100 - DNA, 472 bp, unknown
  OProseR0238 from rose
ctcgttcattcatcaatggaggctgctcggaagctggtgttggtcatggcagcggtgctgctggtggttgcttgcattaatgtagaggtctccgatgcgcagaccatttgcaatgtgtctgtgaataacttgatgtcctgcaagccggccgtaacaaaacctaacccttccaggccgaccaagacctgctgctcggtgctgtcgcacgccgacttgaagtgcctctgctcctacaggaactcgaacctgttgccttctctggggattgaccctaaccttgccatgcagctccctgccaagtgcaagcttcctcaccctgccaattgctagagaggcccttagctacagctacactgtcagatgtggctagctagcttaagttaaataaaagctctatatgtatgattaagaagggattaatcttggagtgaaatgcttaattaatttcctgaaaaaaaaaaaaaaaaaaaaa

SEQ ID NO: 101 - DNA, 634 bp, unknown
  OProse0260 from rose
aattccttcatccatctcatctccttgtctcaccatttcctctccattttccttagttaccaattccttcatccatctcatcttctttgtctctccatttcctttccattttcccaccaattccttcatccatctcatcttctttgtctcaccatttcctctccattttccttagtcaccatttcctacaaaaattccttcatccatctcatcttctttgtctctccatttccttttcattttttatctccattctctagttgcaaagttctgcattttcaagcaaggagaggaagaagaagaaggagccgtgaagatcatatcctccatcatccaccttgaaggcttgcttccaagattcaagatccaaccatctagctctccatctccatctccactcacggtgtaattcgttccttttccttgtaacctttttggtttccttgtttgatttcgtatgaacttgtttctagttaacctaacgtttagggcaaagtttaagcccaatttctatgtttaaataaagttttcgaattctataattgtgattctaagttgcttatgtgagtttgtccgattgaatttgcttgatagacaactgttatatgtgtatctttacaaaaagaaaaaaaaa

SEQ ID NO: 102 - DNA, 649 bp, unknown
  OProseR0277 from rose
gcctctgccctctctgctcttccaaagcttcctcctttcattttaaagtttgcaacttcttcttccaagcattttgaaatggagtcaatgaagatgaagctttcagtggctatgttggtggttgtgatggtggccttgtcaagcattgagaaggtggctgcagctgatgctccagcacctagcccaacatctgatgccgccaccctctttgtgcccactttctttgcatctctagcagctttggcttttggagtcctcttctgaatcagctcatgatcagtcatgactgcattttaggctgattggtgtagtagttatattatgcatttctctatctttctcactcacttttccttctctctctagaatcgcttgtattcagtttttcaggaaagagagagagtggaaatgggttggatcgatattttatcttctttctttcttggggatttcaggttgtaatttgattatttattggtgttgatatgaggaggganttgtttgagtgtgattgatgtatattaattaatcttcttcttcttgtacgggattgtattgtattttcttcctatgatgatctcctaatgcattgtatttgtgctgcatttgttataataaaatcccattattgtttccaaaaaaaaaaaaa

SEQ ID NO: 103 - DNA, 706 bp, unknown
  OProseR0286 from rose
gttattttcatctctagtgacagggaccaagcctcgtttgatgactttttctctggaatgccatggcttgctcttccctttggtgactcaagaaaggcatccttgagtcgccgattcaaggtccaaggcatccccatgctcgtagccattggtcctactggtcaaacagtcacaaaagaagcaatacatctcattatgcttcatggagctaatgcttatcctttcactgaggagcggctgaaagagatagaagcagaatttaaggagatggcaaagaggtggcctgagaagttgaagaatgcactccatgaggagcatgagcttgtgctctctcgccgaagaagtttcatatgtgatgggtgcaacgagaaaggagaggtgtggtcattctattgcgaggagtgtgactttgatctccatccaaagtgtgctttggaggaggagaagggaaccaaaactgatgccaacccggaagaagatcctcatccaaagtgtgctttggaggaggagaagggaaccaaaactgatgccaagccggaagtagatccccatccaaagtgtgctttggaggaggaaaagggaaccaaaactgatgccaatccgggaagaagacccccacgaagatgggtatgccatggtgaattttgtaaaaagcttaaggagcactttgggtgtgatgcctttaaacttatgtactcagcttcat

SEQ ID NO: 104 - DNA, 363 bp, unknown
  OProseR0371 from rose
cttcatccatttcatcttctttgtctctccatttcctttccattttccttctccattctctagttgcaaagttctgcgttttcaagcaaggagaggaagaagaagaaggagccgtgaagatcatatcctccatcatccaccttgaaggattgcttccaagattcaagaccaaccatctagctctccatctccatctcccctcacggtgtaattcattctttttccttgtaatgatcttttgttttccttgtttgaattcatatgaacttgtttctagttaacaataatgtttagggcaaagtttaagcccaatttctatgtttaaataaagttttcgaattctataaaaaaaaaaaaaaaaaa

SEQ ID NO: 105 - DNA, 597 bp, unknown
  OProseR0556 from rose
cctcgtgccgctttgcctctccatttcctctccattttccttagttcttagacatcttccttcacccatctcatcttctttgcctctccatttcctctccattttccttagttcttagacatcttccttcatccatctcatcttctttgtctctccatttcctttccattttcctaaacacatcttccttcatccatctcatcttctttgtctctccatttcctttccattttcctaaacacatcttccttcatccatctcatcttctttgtctctccatttcctttccattttccttttccattctctagttcttagtttggcttttccaggtacagagaagaagaagaagccgtgaggacatatcctccatcgtccaccttgaatagttgcttccaagattcgagattccgaggttcaagctctccatctccatctcccctgacggtgtaatccattgttttcccggtggtgtgatcttgttttttcctctgttggaattcttgtgaagttgtctggtggttacaagggtgtgtaggccagcgtttgacgccgattgattggttagtaggggtgtcgcggcttgccaaaaaaaaa

SEQ ID NO: 106 - DNA, 676 bp, unknown
  OPRoseR0626 from rose
cccttttccttctcaattccaaatctctcagaatttagtttaatcaaaccccggcctcttgctttcccgaacataccaatcaagaagctgcttttgcagacactatggttttgatttcaggtcttcctgatgaagtaatctacgattgtcttattcgtgtcaagtatgatcagtttccgactataacatctgtttctaaaggttggaaatcagaggttgagctaccagagtttcatcgcttcagaaagaatggtggttatggccaaaagctcattgtgatggttcaagcacgtgttcctcccaaccaaggtggtggtgctggtgtcttcaagtgcctgaaagaaccgctgtaccggttcaccgtctgtgagccggattcgggtgattggggagagttcccgccaatttctgttttccccggcgggtctctacccatgttttgccagtttgcagcggttgggaccaacttggtggtggttggcggtttagacccggtgacgtgggcgagctgtaaatcggtcttcgtctacaacttcatgactgccacgtggcggcgcggaaccgatatgccgggtgggtcccgcacattttttgggtgtgcgtctgactctaaccggatggtgtttgtcgccggcggacacgatggcgagaagaatgcattgagatcggcaatggc

SEQ ID NO: 107 - DNA, 674 bp, unknown
  OProseR0763 from rose
gaaaatccaaaatcatggacctcaagctctctaccctcctctgcttttccctgttcttctccactatcctaaccccaactctctctgagctctgcaaccctcaagacaaaaaggttctcttcgaaatcaagacagcctttaacaacccctacatcttgtcctcatggaaatccgacgccgactgctgtaccgactggtacaacgtcgagtgtgatcccaacaccaaccgcatcaactccctcaccatcttcaccgacgaccgcctcaccggccaaatccccgcccaagtcggagacttgccctacctcgaaaccctcgtgctccgcaagctccccaatctcaccggtcccatccagccctccatcgccaagctcaagcacctcaagatgctcaggctcagctggaacggcctctcaggctcagtccctgacttcctcagccagctcaagaacctcaccttcctcgaactcaacttcaacaacttcacaggctccatccccagctctctttctcagctaccgaacttattagcccttcatctagaccgcaaccagctcacaggtcatattcctagctcattcggacgattcgttggcaccgttccagatctattcctctcccacaaccagctcacaggcaaaatcccaacctcatttgctaacatgaactttgac

SEQ ID NO: 108 - DNA, 672 bp, unknown
  OProseR0774 from rose
ctcaaatcacagcaaacgcagtcgatttagagcttttctcttctactacaatcatgcaggccaaggaggaagatgtgagtttgggagccaacaagttcccggagaggcagccgatcggtatcgccgctcaaaccgaagacgagggcaaggactacaaggagccaccgcctgcgccgctcttcgagcccggcgagctgacgtcatggtcgttttacagggccgggatcgccgagttcgtcgccacgtttctgtttctctacatcaccatcttgacggtgatgggcgtcttgaaaggtgaaaccaagtgcaaaaccgtaggtattcaaggcatcgcttgggcttttggtggtatgatcttcgccttggtttactgcacggccggaatctcagggggtcacataaacccggctgtgacttttgggcttttcttggcccggaagctgtccttgacaagggcggtgttctacatcatcatgcagtgccttggggccatagccggagccgccgtggtgaaagcttttacgaacaagggtcatttcttcgagatcaacancggtggcgccaactttgtagcccatggctacaccaagggtagtggtcttggtgctgaaattattgggacctttgtccttgtctacaccgtcttctctgccactgacgccaagcgtagcg

SEQ ID NO: 109 - DNA, 676 bp, unknown
  OProseR0812 from rose
ggaacatttgtgatttgttttacatatccctcatcaattattttcgtgagccccctctctctcaggatttctaggttttctgaaacggattagggtttccattatcttggtcgccgtcccaaaatttcgaatctttaggttttgatttggaggtttcaaaagggggtcttcgaaggaggttgtagatttggtcaaattttattcatttatttgtatcaactgaatgagctaatggagtcaaagggtgggaaaaagaagtctagcagtagtaattcctcattattctacgaagctcccctaggatacagcattgaagacgtcagacctcacggtggaatcaagaaattcagatcagctgcatactctaactgcgttcgaaagccatcctgagttgtgctaggagttcacatagccccttaagatcgttaatactgcaatctaactttagtctagttctctgtcttacttctctcaatcttctctcgttttcctctcgttctcctttcatctggtgttgcgtcatccttcacttcaataattcgagatggctgtaccggtgtctgcaatcggatttgaaggatatgaaaagaggctcgaggtctgtttcttcgagccaggactgtttgctgaccctnatggcatgggtctccgcactttgtncaaagctccaattgat

SEQ ID NO: 110 - DNA, 710 bp, unknown
  OProseR0125 housekeeping gene arginine decarboxylase from rose
ggattcggcacgaggattgggtctcagatccctacgactgctctgctcgctgatggtgtatccgaggcggcgcagatctattgcgaattggtccgcctcggtgcccatatgaaggtcatagacattggaggtggtttggggatagattatgatggatccaaatccagcgactctgagatttctgttggttatgggcttggagagtatgcaatggctgttgttggagcagtccggtatgtttgtgaccggaggtcagtgaagcaccctgtgatttgcagcgagagcggcagagccattgtgtctcatcactctgttctgatatttgaggctgtttctgctagtgcttgtgatgttgctccttccatgtctgcctttgcgcttcagtatttcattgagggacttacagaggaagctcgtgctgattaccggaacctttcggctgcggcgatcaggggagagtatgaagcttgtttgacatatgctgatctgctgaagcagcgctgtgttgagcaattcaaagaagggtctctgggtattgaacaattagccactgtggatgggctttgtgatttggtctcgaaagcaattggagcgtccgatccagttcgtacatacaatgtgcacctctcggttttcacttccattcccgacttctggggcattgggcagcttttccctattgtcccaatcaccggcttgatcaacggncg

SEQ ID NO: 111 - DNA, 1196 bp, unknown
  OProseR0128 housekeeping gene ubiquitin from rose
aattcggcacaaggcggcacgaggtcgaattctacgtcaacctctctctctctctctctctcaaagatgcaaattttcgtgaagaccctcaccggcaagactatcacccttgaggtggagtcttctgacaccattgacaacgttaaggccaagatccaggacaaggaaggaattcccccagaccagcagcgtctgatctttgctggcaagcagcttgaggatggccgaaccctagctgactacaacatccaaaaggagtcaaccctccacttggttcttcgtctgcgtggtggtatgcagatctttgtcaagactcttaccggaaagaccatcactcttgaggttgagagctctgacaccattgacaacgtgaaggccaagatccaggacaaggagggcattcccccagaccagcagaggctcatctttgccggcaagcagcttgaggacggccgcacccttgcagactataacatccagaaggaatccaccctccacttggtgcttcgtctccgtggtggtatgcaaatctttgtcaagaccttgaccggaaagaccatcaccttggaggtggagagctcagacaccattgacaacgtgaaggctaagatccaagacaaggagggtatcccaccagaccagcagaggcttatctttgccggaaagcagcttgaggatggccgcacccttgcagactataacatccagaaggaatccacccttcatctggtgctccgtcttcgtggtggtatgcagattttggtcaagacccttacagggaagaccatcacccttgaggtggagagctcagacaccattgataatgtgaaggctaagattcaagacaaggaggggatcccaccagaccagcagaggcttatctttgccggcaagcagctccaggatgggcgtacccttgcagactacaacatccagaaggagtctacccttcaccttgtcctccgtctccctgggggtttctgaagtctggtgattgcaaagcagcagctgtgaagatcctgaatgttttcttttatgtttttacttttcgcggtacttttattgcttccttgctgatgcaagtattttaatttcatgtttggtattgcctccctgttgaggtacttggacaagttttatttcctatggtacttttggttttataaataaatttggatcttctcctcgtgccgaat

SEQ ID NO: 112 - DNA, 757 bp, unknown
  OProseR0948 housekeeping gene nucleoid DNA binding protein cnd41 from rose
ttcggcacgaggcgccagtctgctctctactcacatcagccacaggtaatacacctggttgctcaagcggcacatcaacttgcatatatgccatacaagaacgagataactccttctccgtgggatactttagcaaagataggttgacactaacgtcaactgactatttcgacgggtttctcttcggttgtggccaaaacaatcaaggcctttttggcgggtctgcagggttattgggcttgggccgcaacaaaatctccctcgtcgaacagagcgcgcaaaagtacagccgcttcttctcctactgcctaccctcaacttccagcgccactggttacctcagcttcggaaaaggcggcggaccttccaacgccgtcaagttcacgccgctatccactgtatctgaaggtgggtccttttacggcctggatgtcgttggaatcaatgtcggcggacgtagggtatcgattcccgcttcagttttttcgtcctcagggacgatcatcgactcagggacggtgataacgcggcttccggcaacagcgtacagtgctttgagggatgcgtttaagcaaggaatgaaaagctatccacaagctgaggagctttcgatactggacacgtgctacgacctgagcgggagcagcacagtgtcgtatcccaaaatagcgttcgctttcagcggtgggttgactctggatttggatgncacaggcatattttacgtggccagtgcttcgcaggtgtgcttggcgtt

SEQ ID NO: 113 - DNA, 2289 bp, unknown
  AJ271698 - Agaricus sequence
gcgatacaccctttctctctctcccgcacccctaggtcctctcacgacatcaacccctcccgtcctcgcttcaagttggcaaccccctccttggttgatatatgcgcctcggtcgatctcagtcttttgctaggcatccaagaggtcgctagtcttgcccttccgaatgatatctcgacagactcggatctctgcctatgtcttcaggatctcgatctttacttggatagtgacaccaggctcgcttggttgacaaacctcatcggccaggacgccttgttgacgtatctgacagctctcatcgacacctcaccatctcgccaagactgcattctgcctgcgcacgcgcatcgtacttgcaatgatcaggacgtctgccactttgattgcgatacaaacttcgtccgttttggtaattcttgcatttgcaatcctccgttctctgtctgtaacggcgtttgcggaaatttcaatgggtgtccatcttccattcctcgaatcggtcgccgcgaaccagagaacgttacaggtattggacacgcaaaatcaggcgtcaagtcgctacacgttgaacgggtcacttgtgtcgacggcgaatgtttttccaagacctgtgccgtggggtggatttattccaaagaggaagacgcctgcattcggagcgcgaccatgtcccgtcgtaataaacgttctgtggcgcgacagcttggagatggcactataacactcgataaccacgtcactatcaacatggtccgccttcttcgggctataggcgaactttatacacattcttcctttctccgcaaacagtctgatcctcgggcacgttcaattatgcggtcttgcgataggcttgaccaactcgctattgatatggtcggatcggacaccctcggttctttccttgacagcacttacgggctcttgaatgttgcacagcttcttgatggattgctctcttccttgaatagcacgggattagaactcttgcaagccgaccttggaactatcgttgcgtcgagcaccaatatcctgaattggtaccattttcctggatctggcgtttctgggtctggcgctcctggatcgaatacgtctggatcgtcctcttctgtcgacttggatactccgattacgatcacgctcaaccgctggcttggtgatctgggacttaatggagtcaaaactactgtcagatacggaggcctcggtggacttggcactgctatcaacggactgttggatggtctcggaatcggcccagctgacgagggccaggcgctccccgcgaacgctagtttcgacgatgctctgcatatgcaaatgggggcgttgttaggctatgccttagacgtgggcagctctgttcccccggccagtcctgctcctcccattggtactgcttcgacaagttcggctcctcaagttggttccgcccccccagctggatctgctgtgactggtgaatctctagacctccaaacgatactagctcctgtctcacgggccactctgggactcttgctggcaacggatctttctgcgcttttggattatgttgacttgatcattgcggcatcgactaatgcccgaacaactctagacctttacgataattttgatggtatgacgactcagttatctctggtgaccaagacggcattggatgtgaagtctctttgctcgaaacaactaggtagtgacgcctccttagcttcaggacgaaaggttgctgcggcaaaggcacctggcgatggttccgtggttatcccgcttgacggtcttttgtacagcctaggtatatctgggaatgtgaccgtcggaggacttggcagaggtttttcggccgctttgaatgggattttgaacgcgttcaaccttggggctactcgggcaaggcgggctggtggtggaagcgccgccgtcaattccgccatcttcatggactcgggtctgacgcaattattccgagcagcagttgagaatgccatagatctggagatgaagacgggttcctcgccgggaccagttgcttcgctattggctggctcgatgtcttcggtgatatcgtcgatgcaggcaatgctcggttctcctacagtagctggactgattggagaagtagacggcttgttgagagcgactggaaagatgaacaggtcgctgacggcttgccattgtacaagcttgcccgtagaagagtctacgggacggtttgttggggctgtgaaggaagttggaaggtggttggatgagcacgcaaggatgagccgtgaagccattttg

SEQ ID NO: 114 - DNA, 885 bp, unknown
  AJ271702 - Agaricus
ccctcgaactcgttcatacttttcctcgagaaaatttacatggcatcctatcccgaactccatgccaggtctcgtcaacctcgttctcgtcaaccacggcccattttaaagtcgctacccgagttcaccccacttccattctcaacctatccgctcgattcccctcatgtccattttccgccaactccaaacctcacttctacgacttttacgcatgccgctggtgtttacgaccgtgcccctataactgtatccgaaaacgtttgcgcgcttcccgaacgtggtgggcgatgttatatgctacagtctcagggtatccctccgtcagggggagtggaggggcggtatgatgattcacggaggagacgagacagtaatgaattcaagggaaattattttcaccctcgcgcattcaaggtttgcgaagttgaacgtgataacaatcatcccgaccgctacgagtcctctccaccaatcccagacttgttgtcaacgcattcttctacctcctcagattcatcctctccgtcaacaccgtcggacttcaaatcagattcatcatatggttctcttactacatcttcgatacccactctctcgtcgaatttgtcactcgacgagacctcatccgtatacccgctagagacatcagtccgtgaccatcacggatgtctaccatggcaagcgtattactcgccatgtaaccaggcaattgtaagttacgtgtgtcccgctagtgtttcggaaagcaagtcgaagaaaacagttcatggaaggaaaatggcaaggtcaaaaggttgtggtgagactgcttcacgactggctaagtttggtgggggattctcagaccaggccattgtattggaaggatgtttgggtggtttt

SEQ ID NO: 115 - DNA, 816 bp, unknown
  AJ271701 - Agaricus
atacagatccccactctccttcgcccccaccagtcttttgagcctcggtctgactaccatctgctatccaaccctttccaaatcctagccatgtttaccttcgcctccttcgctctttttgctctcgccatctcttccgtgaccgctctcgtcgtccctcgaggtcctgacgcccccgctgactggtccagctctctcgaggactatgcgggatatcatgcccgctatttggctatcagctgctacacaaagcacaatgaccctgctttctttgaccgctgctgccatcctcttcagaatggtgcccagccaaacccgtcttgtccgtcaccttcggccgtcgatgacagtttggacaatgatgatgactgcgaggatgactccagttcagcacccaccccaacagcaagtccgactactgcaacaagtcccactccggctccccacaacaattctgctgcaaaggaacaagataccgatgtcgacctcgccgtagatgcccatatcgttcagggcggaatcgcaactttctattatcagcaaggcaatgcgggagcatgcggacactacaacagcgatgatgccctcattgccgccatggactacagaacctacgggaatactagccgcaaatcgcctctttgcggcaaacaagttgagatcacaaatacaaagaaccacaagaaagtagtcgtaaccatcgccgacgcatgtcccacatgcaagaataaaaactctatcgacctttccgttcgtgccttcgaaagaattgccaccaaagacgagggcgaggttcccattacctggaagtatctc

SEQ ID NO: 116 - DNA, 1080 bp, unknown
  AJ271693 - Agaricus
cggcttgcagctggtttcttgcttgcttcctttctcttttctggattttccaatggatgtcccgttagctcaacgctcagtaactcggtggatgatgttgacgatcgcgttttcgtaacgatgcctactgcggctctcagacccgcgtcaggctgctttcctgggttcaatttcaagatgcccgaaagccctcccgcgtccttaactggctggtggtgtgactgggacaccgaatatgcatttgttggttttagctatgaggtttccgcatgccagagtcttcacacgctgaaaactgaattcatggatatcagacagcggtttcacgggcgctatgtacgaatttacggaacttgtgatcggaaaggtttcaatgatgatgtgattaatgcagcatggcacgctgggatcggtgtacacgcactcatttggttcggattcgacggagacaataaatggaaaggccgacgcgacgctttgttctcgacgttacattcgaacccatatgccaaatttgtcgtccgtgtggtccaattcggttctgaacctttgtttgatggcgtcttgagctcgagctccttagcaaaagaagtgtggcgtgcaaaatccgaacttgccaaacttcaaattccagtaactgtcagcgatatggcttattcataccaaaaggatcgcgcttcgagccatgttatagatgccatcgacattattgatgcccatattctgccctttttctctcgggatgcttcgacagcaaaaaaatcctggccactcgtccaacaagatctaaactggtttttgaataacggtcaaggaaagaagatataccttagccagaacggatggccgtcaaaatcataccctggagtagaaccaaacagtgacaaggcggtagccaatgtacaaaatgaaaaggactacttcaatcttcttgatgctcattgctcacatttcaaatcagcgcccggaggaggaataggttggtttgcgcatatatactctgataatcaggaaccgggttatggaatctatgataccaaaggaagactcaaattcccttttagtccccgtgttacttgt

SEQ ID NO: 117 - DNA, 1098 bp, unknown
  AJ271707 - Agaricus
cagttgaagagtgtacggacctttatccgaaacgtaatggaatcaccggacgagttcagcgaatggatacatttctacacatcctcatcaatcatggaaatcatctatggcatgaaagctaaacccgaagatccatatgtcgacaatgccaagaaagccattgagggttttaacgaagctgctgttccaggcaaattcctggttgaaactttccccgtcatgaagcacattccgagttggtttcccggagctgggtggaaaagacaagcactgttttggagagacgttaatcgcgaggtccgtgtcaggccgtttaatctcgtcaaagatcaagtgaatgaaggtactgccacgcggtcggtatgtaggacgttgatcggaaatctgcctgactcaaccgcgcccgaccggattgtgaaagaaaatattgccatcgatacatgcgccgtgtccttcataggcgctgctgaaactagtcattctgctgcccgggtattttttatggccatgcttatgaatccagaagttcaaaagaaaggtcaagctgaacttgacaaggttctcaatggacgtcttccggaaccaaacgacggtccaaatttgccctacataagcgcgatggtgaaagaaacactgcgatggcaacttgtattaccccttgccgttccccatgttgccatcgaagctgatgagtacaatggatactacatacccaaaggtacaattgtctttggaaactcttggacgttcatgcatgatcctgaagtctacaaagacccggaatcgtacatgcctgagcgtttcctgaaagacgggaaactcgacagttctattcgagacccatcgacagctgtattcggttatggtcgaaggatttgtcccggccgatattttgctctgaatgctctctacttgatgatcgcacatacgttggctgttttcgatatcaagcctgcgcttgatgaaaacgataacgagaaagagttcaaggctgatgttacagggggaatgatttcgcagcctgtacctttccagtgcatgattgtgcctcgttcaaaggctgcggctgacttgatacagaacagcgacctcatggaa

SEQ ID NO: 118 - DNA, 423 bp, unknown
  AJ271696 - Agaricus
tcgtcgtcgatccaatcgcccctagacagggtatataaaccactggtttctctaaatcgtatcttcagcatcttcatcttcaacaccatcatcatcatgacttccaacccttcccttaacaacaccgcctctagtgaaccttccaaggtctccggccaacttaactctgccatgggcaccgccaaggagaccgtcggtaaaatgactggtgcgacttctttacaacaatccggcaaggaggaccacgccgctggtgaagctgagaacaaggccgctcaaaccaaaggtcacgccgaaggtatgacggaccaaatcgctggaaagaaggattccatgatcggcgctatgactggcgataagaagcaacaaacttcgggcaatatgcgcgaggacaagggtcaagccgagaagaaaatgaactcg

SEQ ID NO: 119 - DNA, 1916 bp, unknown
  X85113 - Agaricus
ccggcacgagcttgtttcttcagagtttccatccgctctgtctccgcactctcttgaccattccactcttttttttcttttgatttagatgtctcatctgctcgtttctcctcttggaggaggcgttcaacctcgtcttgaaataaataattttgtaaagaatgaccgtcaattctctctttacgttcaagctctcgaccggatgtacgccacccctcagaatgaaactgcgtcctactttcaagtagctggagtgcatggatacccactcatccctttcgatgatgcagtcggtccaaccgagttcagtccttttgaccaatggactgggtattgcactcacggctcaactctttttccaacttggcatcgtccttatgttttgattctcgaacaaattttgagtggacacgctcaacaaatcgccgatacttacactgtcaataaatccgagtggaaaaaggcggcaaccgaattccgtcatccgtattgggattgggcatctaatagcgttcctcctccggaagtcatctccctacccaaagtcactatcacgactccgaatggccaaaagacgagcgtcgccaacccactgatgaggtatactttcaactctgtcaacgacggcggtttctatgggccgtataatcagtgggatactactttgagacaacccgactcgacgggtgtgaacgcaaaggataacgttaataggcttaaaagtgttttgaaaaatgctcaagccagtcttacacgggctacttacgacatgttcaaccgcgtcacgacttggcctcatttcagcagccatactcctgcgtctggaggaagtaccagtaatagtatcgaggcaattcatgacaatatccatgtcctcgtcggtggtaacggccacatgagtgatccttctgtcgccccctttgatcctatcttcttcttgcatcatgcgaacgttgatcgactgattgctttatggtcggctattcgttacgatgtgtggacttccccgggcgacgctcaatttggtacatatactttgagatataagcagagtgttgacgagtcgaccgaccttgctccgtggtggaagactcaaaatgaatactggaaatccaatgaactgaggagcaccgagtcgttgggatacacttaccccgagtttgttggtttggatatgtacaacaaagacgcggtaaacaagaccatttcccgaaaggtagcacagctttatggaccacaaagaggagggcaaaggtcgctcgtagaggatttatcaaactcccatgctcgtcgtagtcaacgccctgcgaagcgctcccgccttggtcaactcttgaaagggttattctcggattggtctgctcaaatcaaattcaaccgccatgaagtcggccagagcttctcggtttgtcttttcctgggcaatgttcctgaagacccgagggagtggttggttagccccaacttggttggcgctcgtcatgcgttcgtccgttcggtcaagaccgaccatgtagccgaggaaataggtttcattccgattaaccagtggattgccgagcacacgggtttaccttcgtttgcagtagaccttgtaaaaccactcttggcacaaggtttacagtggcgcgtgctcttggcggatggaacccctgctgagctcgattcactggaagtgactatattggaggtcccatccgagctgaccgacgatgagcctaatccccgctccaggccgcccaggtaccacaaggatattacacacggaaagcgtggtggttgccgcgaggcttgataggtgttattcattggacattggacttgttgctagaagtatatagataaagttagcgtacatggtttaattgatttaccttgtttgagaaaaaaaaaaaaataatgntannaaaaaaaaa

SEQ ID NO: 120 - DNA, 280 bp, unknown
  SSH03nr023 from Agaricus
tagcgtggtcgcggccgaggtacgccaatcacatgtcatcgttgggcactggtacgcgaactgtgtgtgttcggcgctctcagcgggatgatagtgtatgtaggcgtgaccacgtttcacgcattcgtgacggtgtcctttataattccaccccagtccggcgtaatgccagacctcctgttcgccaaggggggtagttggttgagttagttttccactgttgtcgggttgggccgccatttgtggatagttgtacaagctttttttttttttttttttt

SEQ ID NO: 121 - DNA, 273 bp, unknown
  SSH5nr012 - Agaricus
ttcgagcggccgcccgggcaggtaccgcaggaacagcggcagccgcgtcgcaaaaggtgttcacaaatcgggaacagtcagttgcgggtcccgcagaagtgggtactgcgacgttttcacgtccgcaagtttggcagggcccttcaaagtcaggttgtcttgcacgaggtcggaatgtcttcatggaaatgaagaaagtttcgtcggtgaacatgttaatgtcccaggtcaacccgaggtcaccttcgacgtacctcggccgcgaccacgcta

SEQ ID NO: 122 - DNA, 229 bp, unknown
  SSH5nr019 - Agaricus
tagcgtggtcgcggccgaggtacgagaacggatcccgctcctgtcctgcaggcgactactgctatctaacgcgcaagtccgcacgggcctctagttgtgagcccgaacctattgaacaccagacgtctcccatatgtatgcttcaactctcaccaaagggcatcactcctctggtgcgcgtgctcatgaatcacgatattttgagtacctgcccgggcggccgctcgaa

SEQ ID NO: 123 - DNA, 330 bp, unknown
  SSH07nr010 - Agaricus
tttcgagcggccgcccgggcaggtaccccatttccaggacatcatattttcgcagctgaagttgactttaaccttgagtcgagtagaaatcgtatccggttttgggatatcgttctttgttaccgcgatagtcaaacgattattgtcgcgtttgataatctgctcggtagatactgtgatagtatcattatttctactttgggcacgtccactcctgcggttcccgcgggggcgttgcttgtttgatctttgttgtgccgacatgttaggtgggtaagttggttggttgttagttagttagttaaaagtacctcggccgcgaccacgcta

SEQ ID NO: 124 - DNA, 327 bp, unknown
  SSH07nr032 - Agaricus
tagcgtggtcgcggccgaggtaccccatttccaggacatcatattttcgcagctgaagttgactttaaccttgagtcgagtagaaatcgtatccggttttgggatatcgttctttgttaccgcgatagtcaaacgattattgtcgcgtttgataatctgctcggtagatactgtgatagtatcattatttctactttgggcacgtccactcctgcggttcccgcgggggcgttgcttgtttgatctttgttgtgccgacatgttaggtgggtaagttggttggttgttagttagttagttaaaagtacctcggccgcgaccacgcta

SEQ ID NO: 125 - DNA, 143 bp, unknown
  SSH07nr041 - Agaricus
ttcgagcggccgcccgggcaggtacatgggcttgttagctgccaaagatgctaccagacgcccgaccctatcccttcctgacggaccttcaattgatatgagtggtgttatgttcgcggcgtacctcggccgcgaccacgcta

SEQ ID NO: 126 - DNA, 656 bp, unknown
  SSH07nr083 - Agaricus
ttcgagcggccgcccgggcaggtacagtcgtaaagttgagaagcgctttcgcgaaggcagttctactgtcggcagtagcaaccgaagaagagaaaaatctcgagccagaccaccctccaacggatatcattgttttaacattttttgctctagcttgttcgacgaaatcattgaaggcagcttcgccatccggcagttcaagattttgctgaacatcctcactaggaacagcaaaaccataagtcacgtatgaatatttttcccaaggaatttgacttgcggtgaacttgtcactcgccctcgaaccataccaaccaccataccagccagtgaccagtttactttgtgtttgtgatgttgcagttgcgttggaagcagcagagttgatgccactcgacggtcgttgccgagagcaggttggcgcacaaaagacaaaggaagtgagggcgacaatgacaaggaaggctttcatcgcgaattatagtgtatttattttcacgaatcaatcgtgcgcgtttcaaggagcggatggtatatggttcacgaccagcgtaataagagagncaacggactagcaggagcgagagacgtcnaattgggcgatggggagtgaagagaagagaaacaggtttaatgcccattttgtacctcggccg

SEQ ID NO: 127 - DNA, 369 bp, unknown
  SSH09nr088 - Agaricus
ttcgagcggccgcccgggcaggtaccctcaacttgaatttcatgtaacagcgctggaacatctggtagcacatgccttcgcccaagtatctttttcccgtgttgcatcaattctgcgacagaatgtcgaccatctcgtattcgctcttgaagaaccgaagcaatcaaggccactgcctctgtcttattcaacttgacacctcttgcaagtcgcttttgcgcaataaatccaacctggtggaggaggactttggcctcttcgcgcgggaggagacgcatgacccttttctattttctttcaggagaatgaaaatgggtctgagacaactggtagcagcaaacagaggaaacctcggccgcgaccacgcta

SEQ ID NO: 128 - DNA, 567 bp, unknown
  SSH10nr021 - Agaricus
tagcgtggtcgcggccgaggtacgaaaacgatcgtcacactctgggtcgtagtctggtttctcgaatccctcagatcttaaccgcctcagatctcggcaaacggggcttagggaccaccgaaccacgagatgtttacaatactcttgtcaggcgttatcaggatgtgctgtcctcatctctacaactgcgcgcgttgtcccaggattcttccgttgcggaacccactgtcgttgaaagcttgactacgtataaaaccagtatatcggaatttcaatctgcccttgcatatctgggacgtgacaaaggactggccaattacaataaggatgacaagctcgagactttactcaaggacctggtcaaccttcacaaacacaccctatcctatatcgatgtagtcgtctatcaactcccggtggttggccctatgctcggacccattgtttacgatatcaagtgcatcctggacgaagttcttaacgctgtagaaaacatcagtgacggcctactcaacgtcctcgatcctctcctaataaaaactgtacctgcccgggcggccgctcgaa

SEQ ID NO: 129 - DNA, 235 bp, unknown
  SSH10nr028 from Agaricus
tagcgtggtcgcggccgaggtactcttttattctgcatcggaatttcaacatttgtattgcactagcatcaataggcatcttctatcttcagagatctttttaacgcttccactctccaccatttcgaagccgttaaaaaataccctacacccttttcgtttttccgtgtattaatgtgttgcataaacacaatcgttttcgctttcgttgtacctgcccgggcggccgctcgaa

SEQ ID NO: 130 - DNA, 327 bp, unknown
  SSH10nr080 - Agaricus
ttcgagcggccgcccgggcaggtacagaacataataagtcggtcgtatacgcagcgactgtgtctgcagtatcgagggagttgtcgagagaatatttcgcatcgatctgttgccattcggcgatggtagccagaaacgcggtttgccctcgacgttgtctcgtcgcctcgaataacacagcaccgaaagaaacgatagtcactgaggaaacatgagtgcaacactattattatttttaatggattaatgcgggcctaccatacaccaggcctttcactttggatcgccgatttccatctgaacggtacctcggccgcgaccacgcta

SEQ ID NO: 131 - DNA, 254 bp, unknown
  SSH11nr017 - Agaricus
tagcgtggtcgcggccgaggtaccgtcgcagcttccttgtgtttcccgtgttcaacaagccatctcggcgattctggttggaaaaacatggctatgatgaacaagaggctcggaatcatttggataattaacggaattcgccattgcatttcaccaannnnnggcacgtttttcgacacgctgtagttcacccagaagcccatcatcagaccactgttagtcgtaaattggagtgttccantacctgcccgggc

SEQ ID NO: 132 - DNA, 386 bp, unknown
  SSH12nr023 - Agaricus
tagcgtggtcgcggccgaggtacgcccactgagttcttcacgagtccgcttttcccacctattgaggagaatcgtcttttctccagccaactgcgcgtcaatacgaaactccttaacttcaccaccaccaatccaccgtaagagtttccgcaggccattgcggtcggttacaaggtccatagttccccaaggaaacggatcagcgccttcatttttgcatttctggttgacagccacgagcaatggcaataacgatgcagagggcatcctgtgggtattttgatcgatgataaaaatacccgagtcaggcttgacattgtaaggcatggccctgttattccattcaggagcggagcccggtacctgcccgggcggccgctcccgaa

SEQ ID NO: 133 - DNA, 540 bp, unknown
  SSH12nr053 - Agaricus
tagcgtggtcgcggccgaggtacacagataagcttaaaacaacaagcgactaaattagcgtgataaacgagcaaaggtaagcgctggggtatatgtttcgtagaagtggtcatcgagatgtcagcacgatagacgatgggacttccgcagtattgtagataatgtgaatgtgtcaacgtgcagacggtcgtggcaaaggcatggtttactcttcatcaggctcaccgcttacttgaggctctaatccggctttgagtagctgatctttcagtatccgcatatcatcctgtaatctcgtcacagtctcatgttcgtcatcttgttccgactccatgaactggatttccatcactttcctcgatacctccgaaccgagctcgtcgatctttttctgtgcatcctccagctgcttctgcattgccttcatattcttttcatgctcggcttgcagatcgcccaatttgaggtcgtgagcctcgtgtaattggcgcagcatctcatccgttacaatgccagtacctgcccgggcggccgctcgaa

SEQ ID NO: 134 - DNA, 342 bp, unknown
  SSH13nr021 - Agaricus
tagcgtggtcgcggccgaggtacgtaaaggcgaaagtgcgaccacaagtgaatgtgaccgaacagacgtctaccgaggcgtatagctggttcctctacgggttctagccgcaaccacaaactgatttgggaaaccgaaagacatttacggatagcccttttagatggatggggtttgataacccatcactttattgcgcattatagcaagcgaggcttaaagggaatgggagccatttatatgtatggtgcagcaacttgaatacacatcgcacagtgcaaaaagaccgattggtagtaatattacgcctcccaaaggtacctgcccgggcggccgctcgaa

SEQ ID NO: 135 - DNA, 358 bp, unknown
  SSH13nr086 - Agaricus
ttcgagcggccgcccgggcaggtacggatcttatccgctcctgtcctccagtcggcaaaactggtcaagcgctaatagatgatgctatgttcttttttcaccctgaacattgcattaagccctacgacggttcggtttcccgtattagtcgatgggcgcatcaccgtcgcaagccttccttacaacactctcctgactactaagatcctaacagacacgtgttgacaggagccttcttccaattgagttaggaattagactccgatgatctggcttacagttcataatcaatctaccatacaacaaaaactacgatttccacatacattggtagggtacctcggccgcgaccacgcta

SEQ ID NO: 136 - DNA, 331 bp, unknown
  tomato indicator gene 1Fnr092 - downregulated sequence
acgcggggaccacacaactttcttcttctgctcatcaattagcaattaatccaaaaccattatggctgccaaaaattcagagatgaagtttgctatcttcttcgttgttcttttgacgaccactttagttgatatgtctggaatttcgaaaatgcaagtgatggctcttcgagacatacccccacaagaaacattgctgaaaatgaagctacttcccacaaatattttgggactttgtaacgaaccttgcagctcaaactctgattgcatcggaattaccctttgccaattttgtaaggagaagacggaccagtatggtttaacataccgt

SEQ ID NO: 137 - DNA, 435 bp, unknown
  tomato indicator gene 1Fnr176 - downregulated sequence
cagacaacactatacgcgcagagacagataaatagatagatagatcgatcgatagatcattgatattgttcaaggcaacaggttgcatgtacgcggggagctcaaaaatcaaaatcctatcccctctgctccttcaccaaaaatggagtccctccttcttacctcttcacctgatatcgggaagttgctcagctcctcttcctcctcttcttgtaaatgttatccttcagtttcagcaagtgggcacccttctttcttcctatattttccccacaagaacaaccgaattccctctgttattgtttcagcatcaaacaagaagaagaagaaatctgactcccacagctttattcctaaacctgatgaagccactggtccttttcccgaagctgtgctccttatagagaggaaggtccaagaagatggtatacttgt

SEQ ID NO: 138 - DNA, 255 bp, unknown
  tomato indicator gene 3Fnr355 - downregulated sequence
accatacccgcagcagcaaccttcgcagccatcttcttccacttcaactttagcaaatacaaccaaaacaaattgttcaaaaacctaacaacctaaatattcgaatcatctattgccctcgatagcattaagagtgaatcgacggcaccggtagaggctgagacacggcggagtgatacctccggtaggggggaagaggaatggtgaaagagggaaaaaaaaaaaaaaaaaaaaaaaaatttccctgccgggggg

SEQ ID NO: 139 - DNA, 419 bp, unknown
  tomato indicator gene 7Rnr558 - downregulated sequence
ctgttttacccactccagctcccccgaatagtccgatttttcctccacggcgataaggggctaaaagatctactactttaattcctgtttcaaaaatagataattttgtatccaactgtataaaggcgggcgcagatctatgaataggagacgttgtacgcggggataactaataatattactgattaaatatctcataaaaataagaaaaatgaaaatgatgaaattttttgtgcttgttgtaactattttagcattgttgctaagtgtagcaaatgcacaacaatgtggaagtcaagctggtggagctttgtgtgccaatgggttatgttgtagtcaatatggttattgtggcactactcctgattactgtggacagggatgccagagtcaatgcaactaaattatgttttgtgtgt

SEQ ID NO: 140 - DNA, 80 bp, unknown
  tomato indicator gene 9Fnr046 - downregulated sequence
cgcggggatccctaactaataatctgactgattatctctctcatataaataaaataaatgagaataatgaaattgtgcgt

SEQ ID NO: 141 - DNA, 524 bp, unknown
  tomato indicator gene 9Fnr216 - downregulated sequence
cgcggggatataaaaggatagtggacatcaaaaggttcatattgaaccaaaaaaaagagagaagaagcaatatggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcatgcaggtgtggccaccaattaacatgaagaagtacgagacactctcataccttcctgatttgtctgacgagcaattgcttagtgaaattgagtactcttctagctaattgtttgttgcctattgctgatgtttcatgtgctattcctatcacaactaaagcttgtgatgccttcatttcattaccaccaagtggtggttcatcgttttcgagattttaagcttatgagttctgaaacgcaggcgtatttacttggttcttgaaaaagtgatttacatgtgt

SEQ ID NO: 142 - DNA, 909 bp, unknown
  tomato indicator gene 9Fnr244 - downregulated sequence
ctgtgagttaattccacaagaacaagaatatagtgtctggtcatgaacactatatatgtacgcggggcatccataactaataatattactgattaaatatctcataaaaataagaaaaatgaaaatgatgaaattttttgtgcttgttgtaactattttagcattgttgctaagtgtagcaaatgcacaacaatgtggaagtcaagctggtggagctttgtgtgccaatgggttatgttgtagtcaatatggctattgtggcactactcctgattactgtggacagggatgccagagtcaatgcaactaaattatgtcttgtgtgtacgcggggacacattctcacacacaacattttatatatcctcttttttttttcctaataacatcatgggtgtcactagctatacacatgaaaccacaacaccagttgcccctactaggttgttcaaagctttggttgttgattctgataatcttattcctaagttgatgccacaagttaaaaatattgaggctgagggagatggaagtatcaaaaagatgaactttgttgaaggttcaccaatcaagtacttgaagcacaagattcatgttgttgatgacaagaatttggtgaccaaatattcaatgattgaaggagatgttcttggagacaaacttgagtcaatttcctatgacctaaaatttgaagctcatggaaatggaggatgtgtttgcaagtctataactgagtatcacacaaaaggtgattatgtgttgaaggatgaagaacataatgaaggcaaaaaacaagccatggaacttttcaagattgttgaagcatacctcctcgagaatccttctgtctacgcttaagtgatgaaataagaatcaggtccacacgtgttatataacgtgtttgtgaca

SEQ ID NO: 143 - DNA, 268 bp, unknown
  tomato indicator gene Fnr323 - downregulated sequence
acgcggggacatccataactaataatattactgattaaatatctcataaaaataagaaaaatgaaaatgatgaaatttttcgtgcttgttgtaactattttagcattgttgctaagtgtagcaaatgcacaacaatgtggaagtcaagctggtggagctttgtgtgccaatgggttatgttgtagtcaatatggttattgtggcactactcctgattactgtggacagggatgccagagtcaatgcaactaaattatgttttgtgtgt

SEQ ID NO: 144 - DNA, 169 bp, unknown
  tomato indicator gene Fnr349 - downregulated sequence
acatacatgttcccactttattctagtaatacaaatttatatattttaaacacacaaccacatggatatatattaccttattattatcatagtcttcaagctagaacgtacttatagactggaggtggtggagacttgtatttgtaaacgggtggaggtggagacttgt

SEQ ID NO: 145 - DNA, 413 bp, unknown
  tomato indicator gene 1Fnr224 - upregulated sequence
acttctactggtggctcatttaaatgtgcccaattgctacttatccaccatccatttttcctgattagttgatccaaactttcctcattttccaaattagaaagactttcttccgctgttttagttgcctcctcaaatacaatattcgcctgtttttggtttcgaggggataacgtagagatatatttttgatgctcttcttcaattagtttttttgatctccaatatgcatctatctctttctttgttaatgacttatttgttcgtaatttcatagcttgaggatcagaaacaggagagtcccaacctgccataagagaacccataattatctctttttgttgctaattaacttcttaattaaacttaacacacagttgattaatttcactcaacttgttatatccccgcgt

SEQ ID NO: 146 - DNA, 340 bp, unknown
  tomato indicator gene 3Fnr100 - upregulated sequence
acaatagagcattggctagcaaacaacaacaaaaactaatgtctttgttgtattcatatagcctcatattcatattggtgggtcaatattcattaagatccaacaaaagtccaccacaaatcacttctttcttcttcttccattcttttctggtccaaaatttttgactttttttgagtctttactcacactagttagtagtaggatctgatacgagtgcaacaatatttctgaagagtctgagggatataggctagtagtattcaactgaggtatcagggcaacttccatcagagagaaattctttactgaatggaggaggaatgaatggtggtgaagt

SEQ ID NO: 147 - DNA, 122 bp, unknown
  tomato indicator gene 7Rnr298 - upregulated sequence
acggccttctgcgccgaaattgtggaatcagctgatgcttacttgtttgctggacctatttttaatgactatagctctgttgggtattctctgcttctcaaaagagagaaagctatcattgt

SEQ ID NO: 148 - DNA, 629 bp, unknown
  tomato indicator gene 7Rnr310 - upregulated sequence
acaggctgcagaagggagggtctacgtggatgttggtttctggggaggtttggttccagaaaatgcagaaaatacaagttctctggaaagacttctaaatgcaggggttctgggattgaagtcttttatggtgccatcaggcatcaatgactttcccatgacaactgcatcccacataaaggaggcactcccaactctggctcgatacaaaagacccctacttgttcatgccgaagtgctagtagatcttgatgaaaaagtggagctggaggatggggtagagaatgctagatcatactctacatatctcaagaccaggcctgcttcaatggaagaggctgctatcaatcagctcataacactgtcaaaggacgcgagggctggtggttctgctgaaggagctcatctccatattgttcatctatctgatgctagaacttcactaaacctcattaaggaagccaaacaaaggggtgatagcatcaccgttgaaacttgcccccattatctcgcttttgcagctgaagatattcctgatggagatactcggtttaagtgtgctccacccattcgtgatgcagccaataaagaaaaactatgggatgcgttactggatggagatattgaca

SEQ ID NO: 149 - DNA, 387 bp, unknown
  tomato  indicator gene 7Rnr355 - upregulated sequence
cccacatcaatggtaacgggcaaacaagctgaaggacgaatgccccccagagcagtgtataaagagagcttgcccactggtatccccattccctggcaaccaaggtccccaaggcccagaattcgttctccatcagtaacaacaataacttgaattttcttctcgggccaattttttaatacctcgtgaattttgcctttttctttcaagctgataaaaagaccttgaggacgcctgaagatgctaccatatttctggcatgcttcaccaacagttggagtgtaaactatcggaagaagctcctcaacattgtcaataagaagcttgtagaatagccgctcattcatttcctgaagatccatcatggccatgtatctttgaagtggt

SEQ ID NO: 150 - DNA, 255 bp, unknown
  tomato indicator gene 7Rnr478 - upregulated sequence
gcccgacgtcgcatgctcccggccgccatggcggccgcgggaattcgatttcgagcggccgcccgggcaggtacgcggggaaccaaaatttgtgttctataaaaagttttcatatttagtgatcactaaaaaaaaaatcaagaagatgtcgactactgtaggccaagccattcgttgcaaaactgctgtggcatgggaagctggtaagccattactgatggaggaagtagatgttgctcctccacaaaaaatgga

SEQ ID NO: 151 - DNA, 503 bp, unknown
  tomato indicator gene 7Rnr563 - upregulated sequence
ccaattgactcgaaaacagctacagaggacaatgttttaccagatggaacttttgttaaaaagggaacaagagtaagttatcatccctacgcaatggggagagcagaggaactatggggaagtgattggaacaagtttaaaccagaacggtggttggataaagatgaagttactggaaattggaattttgttactaaagacatgtatacatatcctgtttttcaggcggggccaaggatttgtttggggaaagaaatggcgtttttgcagatgaagagggtggttgctggtgttttacggcggttcaaggtggttccggtcacggaaaaaggggtggaaccggtgtttatttcatacctcacggcgaaaatgaaaggtggtttccatgtgacaattgaggaaagggaccatgagtagtgttgttatttttctacttgtcttaaactattttggatggataaaaaattacttttaattatgaatataataagaatatctggttc

SEQ ID NO: 152 - DNA, 482 bp, unknown
  tomato indicator gene Rnr567 - upregulated sequence
acgcggggatacataaaataaaaatatatatgttcataattgttagaagtggagatgggaaattgtttgagacatggagaatgcgtggcaccaatgacaagtttagatgtagaaaaagaaagattattatggattaatactggtaatagtaagattagagggacaaaaatcagaataacaaagaaggagctagaggacatgttaagaggattgaacatgcaagatgttacggtagatcaagttttatctacaatattgatcaattcaaggagccatggctatcatcatcggaatcaacgtcaatggcgtcccgttctccaaagaattcctgaggtttattgaagaagtaagaagattatcgtttaggttcagaagaaatgaatttaagatttaaattgtaaatgattgataagttagattgagaacatatgtaatagcagaactacttttttgtggtaatgaatgaatgctaattttaaagc

SEQ ID NO: 153 - DNA, 527 bp, unknown
  tomato indicator gene 9Fnr019 - upregulated sequence
acgcgggaagaatataaaatcagcaacaaaatcgaaaattcctccgtacacaacgacgaatacggtgacgacgatagagtaattgacggtagcagtggagctgagagtttacgataatcaaagacgggagctttaatttcagaaaccctagctcatcctaatgcagaatcatccgtacaaattgaaattgatggccaatccgaattcgattcagagtttgctccctcttatcaactttctgaagtaccagtggaatatgaactgccctcgtttggtttctcggaaaaaatcaaggatagaattagtgttaccaaacctggaactttgaatgctcaagcacgaacagaacatcaacgacgtgttcctgatgcagcatcctcattggagctgtcctctttatctgttgcacagtccatttcatcagtgccaagtgcaactctagcagaaagacgatcagcagcagcagtaaattgtattacaggagaagtggtttaacagagttctgacgcccaggttctaactcttgt

SEQ ID NO: 154 - DNA, 388 bp, unknown
  tomato indicator gene 9Fnr248 - upregulated sequence
acgcggggggtgtacgcgctggtgcattaggattactgttgcaatctgttgttctagggtttatgtcacttggggttgaatttttagggaagaagatcggtggtgctaagagactatgggggattttgaacttcgttttagctatttgcttagctatgactattttgataaccaaaatggccgagaaatctcgccggcacgacgccgccggtacactcatgggcccgacgcctggtgttaaaatcggtgccttgcttctctttgccgcccttggtattcctcttgcggtaacttttagtattccatttgctttggcatctatattttctagtaatgctggttcaggacaaggtttgtcactaggagtgctcaatcttgcaattgttgt

SEQ ID NO: 155 - DNA, 633 bp, unknown
  tomato indicator gene 3Fnr244 - constant expression
cgcgggggaggaggaagaaatgggaggaggagaagaagatcaacagaaattgaagaacacagcagcagcagcgtatggctacgagaatgatccgagatgggcagattattggtccaatatactcattcctcctcacatggcttctcgttctgatgtcgtcgaccatttcaagcgcaagttttatcaacgatacatagaccctggtctagtggttgagccaatggctaccagcagttcgtcccaaccagcaaaaccatcagttgctcaaccatcatcatcatcgtccacaccttctaataaccagccgtcacagcgtaactctggtcaagctagtagaacttcaggcacgccaactactccaacttcaaatccgacatcacttcgctgggaccgaaatactatacaattttcagtgaatgcttgggtttttgttgttgcagtcctctcaatatttccgcttacacctgcaaatctgtcgaacagagcatatcgactgtccttcttgggaactgcatgttcttcgctatattcattatactctgtatatgggaaaccgagggcttggaatttacaagcagtgncagtttggttccagtcagttattgtgacgaaggatttcatctccttcattta

SEQ ID NO: 156 - DNA, 216 bp, unknown
  tomato indicator gene Fnr268 - constant expression
atgttacagcccgtctgacctacaagaatgtccctacatggcatagagatctttgtagggtttgtgaaaacatccccattgttctctgtggaaacaaagttgatgtcaagaacaggcaagttaaggctaagcaagttaccttccacaggaagaaaaatttgcaatactatgagatctcagcaaagagtaactagcactttgagaagccttttttgt

SEQ ID NO: 157 - DNA, 408 bp, unknown
  tomato indicator gene L08255 - constant expression
atggaggaggagaaacaccaccaccaccacctgttccaccacaaggacaaggcggaggagggccccgtcgactacgaaaaagaaatcaaacaccataaacatctcgagcaaatcggtaaacttggcactgttgctgccggtgcctacgccttgcatgagaaacatgaggcaaagaaagatccagaacatgcacacaaacacaagatagaggaagagatagcagcagctgctgcagttggggcaggtggatttgcattccatgagcatcatgagaaaaaagatgccaagaaagaagaaaaagcaggtggatttgcattccatgagcatcatgagaaaaaagatgccaagaaagaagaaaaaaaaaagctgaggggggacaccaccatctcttctaaattgttattttag

SEQ ID NO: 158 - DNA, 488 bp, unknown
  pear ACS1-6
aagtgacctttgatcccaaccacttagtgctcaccgccggtgcaacttcagccaatgagacctttattttctgccttgctgaccccggcgaagccgttcttattcctaccccatactacccaggatttgatagagacctcaagtggcgaactggagtcgagattgtacccattcactgcacaagctccaatggcttccaaattactgaaaccgctctggaagaagcctaccaagaagccgaaaaacgcaatctcagagtcaaaggcgtcttggtcacgaacccatcaaatccattgggcaccacaatgaccagaaacgaactctacctcctcctttccttcgttgaagacaaaggcatccacctcattagcgatgaaatttactccggcacggcttttagcaccccatcctttataagcgtcatggaagttctcaaagacagaaactgtgatgagaattccgaagtttggcagcgagttcacgttgtc

SEQ ID NO: 159 - DNA, 456 bp, unknown
  pear ACS2
aactggggttcaaagggatcaaaaggagtgtctggctttagagaaaatgccttatttcaagactaccatggccttttgtctttcagaaaggcaatggcaagttttatggaacaaattagaggaggaagagccaaatttgaccctgctagggtagtcttaacggcgggtgcaactgcagcaaatgagctattgactttcgtcatagctgatcccggtgatgctttgcttgttccaaccccatattatccagggtttgatagagatttgaggtggaggactggtgtgaacattgtgccaattcactgtgaaagctcaaacaacttccagattactcctcaagctttagaagctgcatacaaagaggcagaagccaagaacatgagagtgagaggggtactattcacaaatccatcaaacccacttggtgcaacaatccaaaggacagtccttgaagag

SEQ ID NO: 160 - DNA, 641 bp, unknown
  pear ACS6
ctgctcaaaccttctggatgatgatgaaaagctcagccttagattgctctgccaacgcttcttaggacttttcacttgtgcttgttcttgagctttctttccttgccctacaaatgccctaatccttttgagtgcaacttccactgtgtcatcatccatgttggcgaagcaaaccctaaaccaaccaggctcgacacatttaaatgaagagcccggagaaacgttgagcttcacttcattaacgattacacgccacaacaccatttcgccatcaaacgtttgatctttcaatagcctccttaagtccatccaacagaaaaggccggcattgctcttcaagcagttgattcccacttcctcaagcccatcagtgaagaccccgtgcctctttgcgagcctttttgcgcttgtggtgaggaacttctcgacaaaatcttcgtccgaaagcatggacgcaagcatgtgctgagtttgggatgagaccagcccaaaacttgacatttttcggccaatgttcaccacgtcatcgttgtaggagtaaacgatgccgactctcaatcccgggaaccccatgtccttggacaaactgaagacaatgtggattaggttggggttgcaattcatgttttgtataacctcgg

SEQ ID NO: 161 - DNA, 465 bp, unknown
  pear ACS5-4
tatccaggatttgatcgagatttggggtggcgaacgggagtgcaactgataccagttgcctgtgacagctccaacaatttcaaagtcaccagagcagctttggaagctgcctatgagaaagctcagaaggcaaacatcagagtaaagggcttgctcattaccaacccctcaaatcccttgggcactgtccttgacagagacaccctcagaagtctagtgacattcatcaacgaaaagaaaatccacctagtgtgcgatgaaatctacgctgccaccgtgttcagccagccaagtttcataagcatagccgagatcatagaggaagacattgaatgcaaccgcaaccttattcacattgtgtacagtctttctaaggacatggggttccctggcttcagagttggcattgtatattcctacaatgatgatgtcgttaattgtgcacgaaagatgtcgagttttgga

SEQ ID NO: 162 - DNA, 197 bp, unknown
  pear GAPDH-7
caggttcggaattgttgagggtctcatgaccacagtgcattccatcactggtgagctttgactcgttgagttttgaagttgtaggtttttttataacatgaattgaatttgaaactaacctaagtttatgtttaccttgttattcagccacccaaaagactgttgaggtccatcacagaaggactggagaggtggac

SEQ ID NO: 163 - DNA, 2476 bp, unknown
  pear beta galactosidase
atgggagttggaattcaaacaatgtggagcattctgctattgttttcctgcattttttctgcagcttcggcttctgtgagttacgaccacaaggctataataattaatgggcagaaaaggattttaatttctggctccattcactatcccagaagcactcctgagatgtggccggatttaattcagaaggccaaagatggaggcttggatgttatacagacctatgtgttttggaatggccacgaaccttctccgggaaaatattatttcgaggacagatatgatttggtcaagttcatcaagctggtgcaacaagcaggcctatttgttaatctccggattggcccttatgtttgcgctgaatggaacttcgggggattcccagtttggctgaaatatgtccctggaatcgcttttcgaacggacaatgagcctttcaaggcggcaatgcaaaaatttacagagaagattgtcagcatgatgaaggcagagaagctgtttcaaagtcaaggaggtcctataattctctctcagatagaaaatgaatttggacctgtggaatgggaaattggtgcacctggaaaagcttacaccaaatgggcagctcagatggctgtaggtctagacactggagttccatggattatgtgcaagcaagaggatgcccccgatcccgttattgacacttgcaatggtttctactgtgagaatttcaagccaaataaggactataagcccaaaatgtggacagaagtctggactggttggtatacagaattcggtggggcagttcccactagacctgcagaagatgtggcattttcagttgctaggttcatacaaagcggcggttcgtttttgaactattacatgtaccacggaggaacgaattttggccgaacagccggaggtcccttcatggccactagctatgactatgacgcccccttagacgaatatggactaccccgggaaccaaagtggggacatttgagagatctgcacaaagccattaaaccatgtgagtctgctttagtgtccgttgatccttcagtgactaaactcggaagtaatcaagaggctcatgtattcaaatcagagtcggattgcgctgcattcctcgcaaattatgacgcaaaatactctgttaaagtgagctttggaggcgggcagtatgacctgccgccatggtccatcagcattcttccggactgcaaaaccgaagtttacaacactgcaaaggttggttcgcaaagctcgcaagttcagatgacaccagtacatagtggatttccttggcagtcattcatcgaagaaaccacctcttctgatgagaccgatacaacttacatggacgggttgtatgagcaaataaatatcactagggatactacagactacttgtggtacatgacagatatcacaataggttctgatgaagcatttctaaagaacggaaagtccccgcttcttacaatctcttcagcaggtcatgccttgaatgttttcatcaatggtcagctctcaggaaccgtgtatgggtcgttggagaatcctaaattatcattcagtcaaaacgtgaacctgagatctggcatcaacaagcttgcattgcttagcatttccgttggtctgccgaatgttggtactcactttgagacatggaacgcgggagttcttggcccgatcacattgaaaggtctgaattcaggaacatgggacatgtcagggtggaaatggacatacaagactggtctgaaaggtgaagctttaggcctccatactgttactgggagttcttctgttgaatgggtagaaggaccatcgatggctaaaaaacaaccccttacgtggcacaaggctacttttaatgcaccaccaggtgatgctccattagctttagatatgggaagcatgggaaaaggtcagatatggataaatggacagagcgtgggacgccactggcctggatacattgcacgcggcagctgtggcgattgttcttatgccggaacttatgatgataagaaatgcagaactcattgcggcgagccctctcagagatggtaccacattcctcgatcgtggttgaccccgactggaaatcttttggtggtgttcgaagaatggggcggtgatccgtcagggatttcgttggttgaaagaggtacagccctcgacgcgaagaagctctaggttgaggctgtctgcagctaaagatcgagcagatacgtagattactaaatacgtgaagtggttgtgtacatagacaatctattaattgtcgaaaaaaaatatagctccacatgatatacgaagggttacatacaaagtttgtagtcagtagatttgcgcaagcattttccattgtaagtttgtaacaacttatggaaaagatttccttttcctttacaagaataaatggaaaactaatagagactactttatccttgtctttctaaaaaaaaaaaaaaaa

SEQ ID NO: 164 - DNA, 1764 bp, unknown
  pear polygalacturonase 1
cttgaacacaccaatccttacatttctagctacaattctaagtttccattttccaacatcccatcacattgttcaaaaatattatcagcctcgagttagggtttattatccttcgtgaccctccttttagtatttggttctttttgaaagacgatttgctaggtgtttctaggcctcgtgccatcttgtatctcaccaagagaatatagtaccagccttttacaccaaattaaatagtaaaaagaaagcatcaatggctttaaaaacacagttgttgtggtcatttgttgttgtttttgttgtttccttcagtacaacttcatgttctggtagtagtttccaggaggtcaacgcgcttcatagttacgttgaccatgttgatgatagggtgtccggctacaattctagggcttatccttcatacatggacaccattgaaggtttaaagttcatggaattgatcaggccaagaactcagctctccagttcaaggaagctcaacacaatcaccggtgggatagcaacatcatcagctccggccaaaaccattagcgtcgacgattttggagctaaagggaatggtgctgatgacacacaggcatttgtgaaggcatggaaggcagcttgttcttccagtggagcaatagttcttgtggtaccacagaagaaatatcttgttaggccaattgatttctcaggcccatgcaaatctcaacttacagtgcagatttatggaaccatagaagcatcagaagaccgatcaatctacaaagacatagaccactggctcatctttgacaatgtccaaaacttgctagttgttggtcctggaaccatcaatggcagtggaaacatctggtggaaaaactcatgcaaaagaaaacctcagcccccttgcggtacacacgcccccacggctgtgaccttcaacaggtgcaataacttggtggtgaagaatctgaagatccaagacgcacaacaaatgcatgtcagattccaaaactgcatcaacgtccaagcttcccgtctcacggtaaccgcaccggaggatagccctaatacggacggaattcatgtgacaaatacccagaacatcactatctcgagctcggttataggaacaggtgatgactgtatttctattgtaagtgggtcccaaagagttcaagccacagacattacttgtgggccaggccatggaatcagtattggtagcttgggagaagacggctcaaaagatcatgtttcaggagtgtgtgtgaatggagctaagctttcaggaacctccaatggactccggatcaagacgtggcagggaggctcaggcagtgcaaccaacattgttttccagaatgtgcaaatgaacaatgtcaccaaccccatcatcatcgaccagaactactgtgaccacaaaaccaaagattgcaaacaacagaaatcggcggttcaagtgaaaaatgtgttataccaaaacataagaggaacgagtgcttccggcgacgcgataacgttgaactgcagccaaagtgttccttgtcgggggatcgcgctgcaaagtgttcgactgcagaatggaagagctgaatgcaacaatgttcagcctgcttacaaaggagttgcctcccctagatgttaaaacctagggttcataattatgggcattgtgaaatagattatgcaattcttgtaccaatcagcacataaataattgtttgtttgtcatttttatgtttaaaaaaaaaaaaaaaaaaaaaaaaaaaaaaa

SEQ ID NO: 165 - DNA, 1673 bp, unknown
  pear polygalacturonase 2
cctttcaactactcgttctaaagtaattaagacaagtagcctctttatttctcctacatctccatctcactcctttttcaaatcagaaaatcctaaaaccagccagcacaaatggcaaaccccaaaagcctctcatatccagcagctgcagtttttgcgttgttgatgatggctataagcattactaatgtggatgctgcagccgtcactttcagtgtgagcagtttaggagccaaagcagatggcagtactgactccaccaaggccttcctctctgcgtggtccaatgcttgtgcctccgtcaaccctgctgtcatatatgtccccgcagggaggttcttgcttggcaatgccgtgttctctgggccatgcaagaacaacgccatcaccttccgcattgccggcactctcgtcgccccgtctgattaccgggtcattggaaatgccggtaactggcttctctttcagcatgtcaatggggtcacgatttccggtggagttctcgacggtcagggcaccggattgtgggattgcaagtcctcgggcaagagttgccccagcggagcaactacactgagcttttcgaactccaacaacgttgtggtgagtggattaatatcactaaacagccaaatgttccacattgtcgtcaacggctgccaaaatgtgaaaatgcaaggtgtcaaggttaacgcggccggcaacagccccaacaccgatggcatccatgtccaaatgtcatctggagtcaccattctcgactccaaaatttcaaccggtgacgactgtgtctcagttggccccggcactaccaatttgtggattgaaaacgtcgcatgtggacccggccacggaatcagcattgggagtttagggaaggaccaacaagaagccggtgtacaaaatgttacagttaaaacagttacattcactggtactgaaaacggcgtcagaattaagtcttgggggagacctagcactggatttgctaggagcattcttttccaacatattgtgatgaccaacgttcaaaatccaatcgttattgatcaaaattactgccctaatgacaaaggttgccctggccaagcttctggagttaaggtcagcgatgtgacgtatcaagacattcatggtacatcggcgacggaagtggcggtgaaattcgattgtagttccatgtatccttgcaacgggatcagactgcaagatgtgaagctcacttacaataaccaagcagctgaagcttcctgcatccatgcaggcggaacaactgccggtacggttcagccgacaagttgtttctaactcgagttgtagttttttccatctactcctcctcactcggagtctcgtagtactagttgggataaaaaagaagggactagtcatactataaactatatatatatatatatatataagaattaaagaatatttctagagtagtaggtctaggtctagctctagctctacgtagttgatgtattgagatgtattttgcttgagcctgccgtgttggcagcctattgggcttccttagagcctggcgctgcatcatccaaacccacttcatggagagattctcttttgcattgggtgctttgtattatggaatgttgtaacttgaaagtgataaatgcaatatgaattaaaagtaaaaaaaaaaaaaa

SEQ ID NO: 166 - DNA, 1190 bp, unknown
  pear actin
ggtgtcatggttggtatgggtcagaaggatgcctatgtaggggatgaggctcaatcaaagagaggtatcttgaccttgaaatatccaattgagcacggtattgttagcaattgggatgacatggagaagatctggcatcacacattctacaatgagctccgtgtggctccagaagagcatccagtcctcctgacagaagcaccccttaaccccaaggccaatcgtgaaaaaatgactcaaattatgtttgagacattcaacaccccggctatgtacgttgcaattcaggctgtcctttccctatatgctagtggacgtacaactggtattgttctcgactcaggagatggtgtgagctacacagttccaatttatgaagggtatgccctcccacatgccatccttcgtttggacctggcaggtcgtgatcttacagatgctctcatgaaaattttgactgaacgtggttactctttcaccacaactgctgagcgggaaattgtgagggacatgaaggagaagttagcctatattgctcttgactatgaacaagagctagagacatcaaagaccagctcgtctgtggagaagagttacgagctacctgatggacaggtaatcactatcggagctgagagattccggtgccctgaagtcctcttccagccatccatgattggaatggaagctcccggtatccatgaaaccacatacaactctatcatgaagtgtgatgttgatattaggaaggatctgtatggaaatattgtcctcagtggaggtcccactatgttccctggaattcgtgacaggatgacgaaagagattacagccttggctccaagtagcatgaagattaaggtcgtagcaccacctgagaggaaatacagagtctggattggaggctccattttagcgtccctcagcaccttccaacagatgtggattgcaaaggcagagtacgatgagtctggcccctcaattgttcacaggaaatgcttctaatcgcgcaaacaaagcaagcggggaacaaagaatctacacaacagattttgtactacagaaaggcttcttctattcaagcggttgcggaatttctttttgtattcattttcgaatttcattcatgtcgatttctctgtgtttgttatgacgggacatgagcatggaggcgagcaaattatgattaa

SEQ ID NO: 167 - DNA, 1384 bp, unknown
  pear beta xylosidase
atttcgtgtgaaatgtgcaggttagtaagcaggacttggaagacacatataacgtgcccttcaaagcctgtgtggtggatgggaatgttgctagtgttatgtgctcctacaaccaggtcaacggaaagcctacttgtgctgaccctgatctcctcaaaggcacaatccgtggtcaatggaaactcaatgggtgagtcccattgatttgaattatcacttgaattgcttatgtataattttcttaatttatatttgtagcctctaattaattactgattaagcatttggtttatgtgtccaaaataggtatatcgtctcggattgtgattcagttggtgtttattatgataaccaacattacaccaaaacaccagaagcagcagctgcatacgcaattaaagcaggtcattagcgatttaaccattttatatgagtttaatgttatctagtgcgtctcaaaagaggaagttaagaaaatctaagcccaattttgtaataagcaggtttggatttggattgtgggccattccttggcatccacacagaggcagccatgaggactggccaggtaaacgagatcgacattaactatgcgttggctaacacaatcacggtccagatgagattgggaatgtttgatggtgaaccgtcaactcaacgatatggaaacttaggcctagcagatgtgtgcaaaccatccagcaatgagctagcccttgaagctgccagacaaggcattgttctgcttgaaaaccgtgggaattcgctacccctctcaaccatacgccaccgtactgtggcagtaatcgggcccaattctgatgttacagaaacaatgattggaaattatgctggtaagtctactaaaaatgtaaacctgtacatatatatatattttgtcactctagtccaatttcttagtaaagctttaaaatttgatccattggatatttttgtaattgtatgtaggagttgcatgtggttacactacgccactacaaggaattgcaaggtacacaaggaccatacaccaagctgggtgcacagatgttcattgcaatggaaatcaactaattggtgctgctgaggttgcagctagacaagctgatgcaaccgtcttagtaataggccttgaccaatcaattgaagcagagttcagagaccggactaaccttctcttgcccggacaccaacaagagctggtgtctagggtggccagggcttctagggggccgaccattctggtcataatgtcaggcggccctattgatgtgatgttcgcaaagaatgaccctcgcattggagcgattatttgggttgggtatccaggccaagctggaggaaccgccattgctgatgttctattcggcactacaaacccaag

SEQ ID NO: 168 - DNA, 1153 bp, unknown
  pear expansin 2
ctcaatacttcctcctcctcctctctctccttagtttctctctctaaaaacgcaaacaatggcttttacttctcactccaccattgctcttctgttctttgtcctcaatctatgtcttcaaggtacttttggtgactatggaggtggatgggagggcggccatgccacattttatggtggtggtgatgcctctggcacaatgggaggtgcttgtggatatgggaacttgtacagccaagggtacggaaccaacactgcagcactgagcacagctctcttcaacaatggcttgagctgcgggtcttgctatgagatgaaatgtggcagtgaccccaaatggtgcctccccggcagcatcatcgtcaccgccaccaacttctgccctcccaactttgcgcaggccaacgacaatggtggctggtgcaaccctcctctccagcactttgatttggctgagcctgctttcttgaaaattgcccaatacagagctggaattgtccccatctccttcagaagggtttcgtgtgtgaagaagggaggaataaggttcacaatcaacggccactcttacttcaacttggttttgatcacgaacgtcggaggagcaggagacgtccactctgtgtcgatcaagggttccaagacagggtggcaacccctgtcaagaaattggggccagaactggcagagcaactcttacctcaacggccagagcctctccttccaggtcaccaccagcgacggcagaactctcaccgccaacaatgttgcgccgggaaactggcagttcggacaaacatttgagggcagtcaattctgagactcctccggcggtaaaacagttaaaaagtctggtgaatggtgaatggtgaatggtgaagggtgaattgtatatggtaatttgtacgtggatgggaagaggagagtttggggggtagttttaggagaggcaactgattgctgaggtggctaactggcacccgctagtcctatatatataaataataataataatatttatttatatatagtgaggagtttgtgagtttatagttaatttattaaaaatttgttaatcataagtagagggacatgtaagttctcgtatgaaaggaacacaagtactatcatttccaaaaaaaaaaaaaaaaaa

SEQ ID NO: 169 - DNA, 1118 bp, unknown
  pear expansin 3
gccctacacaaaaactaaaactcctctctttcttttccctattgaaatcaaaacccaccaaaaagccacaaaaatggcagctcatgcattgtcttttgctcctatagccctctctgttgttctctttaatctacatctgcatggtgtatttgctgtttatggtagctgggaaggcgctcatgccacattttacggtggcggtgatgcttctggcacaatgggaggagcatgtggttatgggaatttgtacagccaggggtatggaaccaacactgcagctttgagcacagcattgttcaacaatggcttaagctgtgggtcttgttatgaaatgagatgcgacaatgacccgagatggtgccgtcctggatccatcattgtaactgctacaaacttttgccctcctaactttgctcagtccaacgacaatggcggatggtgcaatcctcctctccagcatttcgatttggctgagcctgctttcttgcaaattgcccaataccgtgctggaatcgtgccggtttccttcagaagagtaccttgtgtgaagaaaggaggaataagattcaccatcaacggccactcctacttcaacctggttttgatcaccaacgtggctggggcaggagacgtccattcagtttcaatcaaggggtccagaacagggtggcaacccatgtcaagaaactggggtcaaaactggcagagcaactcttacctcaatggccaagccctctccttccaagtcaccaccagtgacggtagaaccgtcacgagctacaacgtcgcgcctggtaattggcagtttggtcagacattctccgggggtcaattttagagatattcctctacattattggtaaaaatttgtatatctatctgtcatttttttcccgtaaacttttctgagtgtaaaagcaaagagtagttgtgaagtggaggtttgctgaggtgagctaaaaaaacacccgctgggcctttcacatttgagttttcctggagaaatgatattcacctcattcaggttgtaaccaatttctcagttgtacttgtaaccttaatgatatatatatttataaaaaacgagaaagctttatcaaaaaaaaaaaaaaaaaaaa

SEQ ID NO: 170 - DNA, 1355 bp, unknown
  pear expansin 5
atttcctctcccctgttttttcaagcctcattcgtttccaatccaaatgaaaatggcttcttcatctgggtttttcatggcgggtcttcttgcaatgttggtggcgtctgcgcatgcttacggtggaggcgggtgggttaatgctcgcgccactttctacggcggcggtgacgcttctggcacaatgggtggagcttgtggttatggcaacctctacagccaggggtacggaaccaacacggccgcgctgagcactgctctgttcaacaatgggctggggtgcggatcttgctatgagattaggtgtgtgaacgaccccaaatggtgtctgccgggctccattgtggtcaccgccaccaatttctgcccgccgaacaacgccctccccaacaatgccggcggctggtgcaaccctccccagcaccacttcgacctctctcagcctgtcttccagcacattgcccaatacaaagccggagttgtgcctgtgtcctacagaagggtaccctgcatgagaaggggaggcatcagattcacaatcaacggacactcctacttcaacctggtccttatcacaaacgttggtggtgccggtgatgtgcactctgtttccgtcaaagggtccagaactggctggcaagcaatgtcccgaaactgggggcaaaactggcaatccaacagctacctcaacggccaagctctctctttcaaggtcacaacaagtgacggcaggactgttgtgtcctacaatgctgcccctgctggatggtcctttgggcagacctacagcggtgcccaactccgctagggcacccccacgccacctcctaatttaccctccatatttagccatttcctaggtcattaaatatatactgaagtataattaagattagtgtgactcttaagtatactaggatattagttagtattctgagtcactgagtcaaggtctttggcctctgcggtcaaaagtgagtagggcttattttataagggctctcccatcaagttttaatttttgaattatatgtgtcttaggtgtagggtaccctactatctttgggaccctaactccttgggtattgtttctttttacttctgcagtatttgtaaaatcagaagttggcagaggtggtggactttgaccacccgccattagtgttttgaaatattactagcatcttgggcttgttttggaagggcattattgtaattcagctgaggctgatggttaattcagtgttgtccgtggttgttatatttgaatgttgttgaaggactaaatgtaaattttcctttctctactaagtaatggattttctaatttactatttaaaaaaaaaaaaaaaaaa

SEQ ID NO: 171 - DNA, 1272 bp, unknown
  pear expansin 6
catcactctctctcctctctctgctttctcactcccctttctctctctccggcaatggcctcccttcgcgtgctctacattgctttcatgctctcactcttcatggcggccaacgctagaattccaggagtttacaccggtggctcatgggagggcgcccacgccaccttctacggtggcaacgacgcctctggaaccatgggtggcgcttgcgggtacggaaacctctacagccaaggctacggcgtgaacacggcggcactgagcactgctctgttcaacaatggccttagctgcggcgcctgcttcgagattaagtgcggcgacgaccccaggtggtgccacccaggcaacccctccatcttagtcaccgccaccaacttctgccctcctaacttcgctcagcccagcgacgacggcgggtggtgcaaccctccccgcacccacttcgacctcgccatgcccatgttcctcaagatcgccgagtacaaggccggcatcgtccccgtctcttaccgccgagttccgtgcagaaagcaaggcggagtgagattcacaattaacggtttccgttacttcaacctggttctgatcaccaacgtcgcgggcgcaggggatatcgtgagggtgagcgtaaaaggcgggaacactgggtggatgccgatgagtcgcaactggggacaaaactggcaatccaacgcagacctggtgggccagaccctgtcgtttcgagtcacgggcagtgacaggcgcacatccacctcccacaacgtggcacccgctgattggcagttcggacaaactttcaccggcaagaatttccgggtctaaaattaagaagggaaaaaaaagtttatccactatctttaattttccttttgggtttttaactttttttttaaattatcaaagtttaatttcccgccatctgattttccttaattttcccgggaaaatttggaagcggtgggagtataaaagtaaagtgttagatgatatggggttaaaagttaaaattgggtggtaagataggtcgaaaagcgacttcttttgcaagtgtggtgtgcggcaactttttacttttggtgctttttttttttaggtttgagtgggaggctggtaaaaatttaggtgatccggccaaatagtgcgtgtaaaaggagtcgaagcggctgcaaagaaccaacatgcagcctgcagctctacccatatcttttctagaattttatatgatatatatttcagttagtataaaaaaaaaaaaaaaaaaaa

SEQ ID NO: 172 - DNA, 560 bp, unknown
  rose OProseR1663 (Unknown function)
angattaccccattctatttangggtgacactatagaatactcaagctatgcatccaacgcgttgggagctctcccatatggtcgacctgcaggcggccgcgaattcactagtgatttcgagcggccgcccgggcaggtggcagcggtgctgctggtggttgcttgcattaatgtagaggtctccgatgcgcagaccatttgcaatgtgtctgtgaataacttgatgtcctgtaagccggccgtaacaaaacctaacccttccaggccgaccaagacctgctgctcggtgctgtcgcacgccgacttgaagtgcctctgctcctacaggaactcgaacctgttgccttctctggggattgaccctaaccttgccatgcagctccctgccaagtgcaagcttcctcaccctgnccaattgctagagaggcccttaatctacagctacactgtcagatgtggctagctagctgaagttaaataaaagctctatatgtatgtatgattaagatgggattaatcttggagtgaaatgcttaattaatttccaaaaaaaaaaaaa

SEQ ID NO: 173 - DNA, 810 bp, unknown
  rose OProseR0948  - HHG4 nucleoid DNA binding proteint cnd41
antnanttacngcgaattcnctngngacctgnncgcntctacataaagggaacaaaatgctggagctccaccgcggntggcggccgctctagaactagtggatcccccgggctgcaggaattcggcacgaggcgccagtctgctctctactcacatcagccacaggtaatacacctgggttgctcaagcggcacatcaacttgcatatatgccatacaatacggagataactccttctccgtgggatactttagcaaagataggttgacactaacgtcaactgactatttcgacgggtttctcttcggttgtggccaaaacaatcaaggcctttttggcgggtctgcagggttattgggcttgggccgcaacaaaatctccctcgatcgaacagagcgcgcaaaagtacagccgcttcttctcctactgcctaccctcaacttccagcgccactggttacctcagcttcggaaaaggcggcggaccttccaacgccgtcaagttcacgccgctatccactgtatctgaaggtgggtccttttacggcctggatgtcgttggaatcaatgtcggcggacgtcgggtatcgattcccgcttcagttttttcgtcctcagggnacgatcatcgactcagggacggtgataacgcggcttccggcaacagcgtacagtgctttgagggatgcgtttaagcaaggaatgaaaagctatccacaagctgaggagctttcgatattggacacgtgctacgacctgagcgggagcagcacagtgtcgtatccccaaaatagcgttcgctttcagcggt

SEQ ID NO: 174 - DNA, 478 bp, unknown
  rose OPRoseR0049 (actin)
tcggcacgaggggaaatcactgctcttgctccaagcagcatgaagatcaaggttgtggctccaccagagaggaagtacagtgtctggatcggagggtccattcttgcttccctcagtaccttccaacagatgtggatttctaagggagagtatgatgagtctggtccatccattgtccacaggaagtgcttctgagttctataaatgttttggtggtgagttattttttcttcaatttagttggattttcgtgtcaagtgtcatgtggttgggatgtttgtagtggagagtgattgggatctttttttttccgtccttaattccctttttgttctatatttgacggttctttttctttcttttttcttgggaacattaatattactagctttattgtatctgaatgatagtgtcagtttgtggtcataaactgctatggaaattatatttaaataaaaaaaaaaaaaaaaaaaaaaac

(19) United States
(12) Patent Application Publication
(10) Pub. No.: US 2013/0005607 A1
(43) Pub. Date: Jan. 3, 2013

(54) QUALITY CONTROL OF AGRICULTURAL PRODUCTS BASED ON GENE EXPRESSION

(75) Inventors: Monique Francisca VAN WORDRAGEN, Arnhem (NL); Rudolf Aart De Maagd, Wageningen (NL); Jurriaan Johannes Mes, Wageningen (NL); Peter Albert Balk, Zetten (NL)

(73) Assignee: Nsure Holding B.V., PD Wageningen (NL)

(21) Appl. No.: 13/610,093

(22) Filed: Sep. 11, 2012

Related U.S. Application Data
(63) Continuation of application No. 13/110,790, filed on May 18, 2011.
Application No. 13/110,790 is a continuation of application No. 12/376,757, filed on Feb. 6, 2009, now abandoned.

(30) Foreign Application Priority Data
Aug. 7, 2006 (EP) 06118502.1

(51) Int. Cl. C40B 30/04; C40B 40/06; C07H 21/04; C12Q 1/68

(52) U.S. Cl. 506/9; 435/6.12; 506/16; 536/24.3; 536/24.33

(57) ABSTRACT

The invention relates to the field of quality testing of fresh plant-based and mushroom based products. Methods, carriers and kits for determining the quality stage are provided.

### FIELD OF THE INVENTION

The present invention relates to the field of quality testing of fresh plant-based and mushroom-based products, such as food or feed products and ornamental products. Provided are methods for quality testing and quality prediction and diagnostic kits for quality screening and selection of high quality products. In particular, relative or absolute mRNA expression levels of defined sets of gene transcripts are determined, whereby a specific stage or category of a quality trait is determined and an advice for subsequent distribution or processing chains is given. Thus this invention describes a new support tool for stakeholders in agro-production, agro-distribution and agro-processing.

### BACKGROUND OF THE INVENTION

Fresh plant and mushroom products that are generated in agricultural production chains differ in intrinsic quality (phenotype) in a batch-dependent way. Partly this is due to differences in growth conditions, but even after harvest the products are actively metabolizing and responding to environmental triggers, such as temperature, light, humidity, etc.

The batch quality has a strong influence on the type of application that the product can have in downstream distribution and/or processing chains. Batch quality is the main parameter in decisions concerning (international) market choice. In addition high quality batches can be assigned A-status, which will increase the added value that can be obtained. At the moment these batch-to-batch differences in quality are only marginally determined and consequently hardly exploited. Present quality tests usually involve measurements of a physiological parameter such as color, firmness or pH that is always secondary in nature and gives no information on the nature or status of the biological process that is causing the effect.

Another problem associated with present quality tests is that no prediction of future quality can be made, because the tests only allow the present status to be determined.

The present inventors found that genomics technology offers a complete new spectrum of possibilities to assess the quality of fresh agricultural products during all stages (from production, to harvest, to processor or consumer) of the diverse production chains in which they are used. At present in plant production, genomics technology is only used for generating scientific knowledge and for breeding purposes. However, the present inventors found that the high information content of genomics data, makes it eminently suited for use in quality diagnostics. They have shown that genomics-based agro-diagnostics even based diagnostics even allows prediction of future quality and can thus be used as support tool, for decisions concerning applications, treatments or destinations for specific batches.

The herein provided genomics based diagnostic methods and kits facilitate the implementation of precisely controlled agricultural production and distribution chains, and allow for batch differentiation at auctions, before storage and for processing industry. Robust quality assays are provided herein, which were developed based on a combination of expertise in molecular biology, post-harvest physiology, chain knowledge and quality dynamics modeling.

### DETAILED DESCRIPTION OF THE INVENTION

### General Definitions

The term “gene” means a DNA fragment comprising a region (transcribed region), which is transcribed into an RNA molecule (e.g., an mRNA, or RNA transcript) in a cell, operably linked to suitable regulatory regions (e.g., a promoter). A gene may thus comprise several operably linked fragments, such as a promoter, a 5′ leader sequence, a coding region and a 3′nontranslated sequence comprising a polyadenylation site.

“Indicator genes” refers herein to genes whose expression level is indicative of a certain quality stage of a fresh agricultural product.

“Expression of a gene” refers to the process wherein a DNA region which is operably linked to appropriate regulatory regions, particularly a promoter, is transcribed into an RNA molecule.

“Upregulation” of gene expression refers to an amount of mRNA transcript levels of at least about 2 times the level of the reference sample, preferably at least about 3×, 4×, 5×, 10×, 20×, 30× or more.

“Downregulation” of gene expression refers to an amount of mRNA transcript levels of at least about 2 times lower than the level of the reference sample, preferably at least about 3×, 4×, 5×, 10×, 15× lower.

“Constant” refers to an essentially equivalent mRNA transcript level as in the reference sample. Generally, housekeeping genes (such as glyceraldehydes-3-phosphate dehydrogenase, albumin, actins, tubulins, 18S or 28S rRNA) have a constant transcript level.

“Relative” mRNA expression levels refer to the change in expression level of one or more indicator genes relative to that in another sample, preferably compared after “normalization” of the expression levels using e.g., housekeeping genes. The fold change (upregulation or downregulation) can be measured using for example quantitative real-time PCR. The fold change can be calculated by determining the ratio of an indicator mRNA in one sample relative to the other. Mathematical methods such as the 2(-Delta Delta C(T)) method (Livak and Schmittgen, Methods 2001, 25:402-408) or other mathematical methods, such as described in Pfaffl (2001, Nucleic Acid Research 29:2002-2007) or Peirson et al. (2003, Nucleic Acid Research 31:2-7) may be used.

“Absolute” mRNA expression levels refer to the absolute quantity of mRNA in a sample, which requires an internal or external calibration curve and is generally more time consuming to establish than relative quantification approaches.

The term “training sample” or “training batch” refers herein to a reference batch of the same type of plant material (e.g., same tissue type and cultivar), having a predetermined quality status (i.e., the expression profile of indicator genes of the training batch is correlated with the quality stage or predicted/future quality stage). The expression profile of the indicator genes in a “test batch” can then be analyzed and thereby correlated with the quality stage (or predicted/future quality stage) of one of the training batches.

The term “substantially identical”, “substantial identity” or “essentially similar” or “essential similarity” means that two peptide or two nucleotide sequences, when optimally aligned, such as by the programs GAP or BESTFIT using default parameters, share at least a certain percent sequence identity. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). It is clear that when RNA sequences are said to be essentially similar or have a certain degree of sequence identity with DNA sequences, thymine (T) in the DNA sequence is considered equal to uracil (U) in the RNA sequence. Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA. Or using in EmbossWIN (version 2.10.0) the program “needle”, using the same GAP parameters as described above. For comparing sequence identity between sequences of dissimilar lengths, it is preferred that local alignment algorithms are used, such as the Smith Waterman algorithm (Smith T F (1981) J. Mol. Biol 147:195-7), used e.g., in the EmbossWIN program “water”. Default parameters are gap opening penalty 10.0 and gap extension penalty 0.5, using Blosum62 for proteins and DNAFULL matrices for nucleic acids.

“Stringent hybridization conditions” can also be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g., 100 nt) are for example those which include at least one wash in 0.2×SSC at 63° C. for 20 min, or equivalent conditions. Stringent conditions for DNA-DNA hybridization (Southern blots using a probe of e.g., 100 nt) are for example those which include at least one wash (usually 2) in 0.2×SSC at a temperature of at least 50° C., usually about 55° C., for 20 min, or equivalent conditions.

The term “comprising” is to be interpreted as specifying the presence of the stated parts, steps or components, but does not exclude the presence of one or more additional parts, steps or components. A nucleic acid sequence comprising region X, may thus comprise additional regions, i.e., region X may be embedded in a larger nucleic acid region.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

The term “plant” refers to any organism of which the cells, or some of the cells contain chloroplasts. It may refer to the whole plant (e.g., the whole seedling) or to parts of a plant, such as cells, tissue or organs (e.g., pollen, seeds, gametes, roots, leaves, flowers, flower buds, anthers, fruit, etc.) obtainable from the plant, as well as derivatives of any of these and progeny derived from such a plant by selfing or crossing. “Plant cell(s)” include protoplasts, gametes, suspension cultures, microspores, pollen grains, etc., either in isolation or within a tissue, organ or organism.

“Mushroom” or “fungus” refers to members of the kingdom Fungi, including parts thereof, such as hyphae, fruiting bodies, spores, etc., as well as progeny or derivatives thereof. Generally throughout the description, reference to plants above will equally apply to mushrooms and it is understood that, even if mushrooms are not mentioned, they are encompassed in the embodiments.

The term “batch” refers to a collection of harvested plant or mushroom products that share a considerable part of their history in the production and or distribution chain. For example the term “batch” is used to describe a group of plant or mushroom products grown in the same greenhouse in the same period and harvested at the same time.

The term “quality trait” refers to a specific physiological characteristic of a plant or mushroom product that is important for determining the economic value. For example the term may be used to refer to color or taste or firmness or tenability of a product.

The term “quality stage” or “quality trait stage” refers to a predefined moment in the development of a quality trait, described by a specific set of physiological of morphological characteristics. For example, the term may be used to refer to a specific level of firmness of fruit, defined by the level of resistance to penetration by a metal rod. Or it may refer to a specific level of ripeness of tomato, determined by the color of the fruit as compared to a standard color-card. “Predicted or future quality (trait) stage” is the quality stage which is predicted to develop in the batch after time, as determined by the expression profile of a set of indicator genes. This is encompassed by the broader term “quality stage.”

The term “fresh” refers to plant products that have not (yet) been processed, or only minimally processed (e.g., cut or sliced and/or packaged) after harvest and which are still actively metabolizing and responsive to the environment.

“PCR primers” include both degenerate primers and non-degenerate primers (i.e., of identical nucleic acid sequence as the target sequence to which they hybridize).

“Oligonucleotides” refer to nucleic acid fragments suitable for use as PCR primers or hybridization probes, e.g., coupled to a carrier in a nucleic acid microarray.

“DNA Microarray” or “DNA chip” is a series of known DNA sequences (oligonucleotides or oligonucleotide probes) attached in a regular pattern on a solid surface, such as a glass slide, and to which a composition consisting of or comprising target sequences are hybridized for identification and/or quantification.

A genomics-based method, exemplified by six specific examples, is provided that can be used for measuring (and predicting) specific quality characteristics (or quality stage) of a fresh product. The tests are based on the combined expression profiles of a carefully selected set of indicator genes. In living organisms, each developmental step and every interaction with the environment is orchestrated by DNA encoded genes. The history and actual condition of a plant, animal or microorganism is accurately reflected in the activity profile of its genes. The indicators were selected by combining gene expression analysis (using microarrays) and thorough physiological analyses with knowledge of distribution chain logistics. The information was used to select those genes that are most strongly correlated to the trait(s) of interest. The selected set of indicator genes was translated into a reliable and robust assay for use in practice.

Quality assays and kits are provided for
(1) the determination/prediction of fungal incidence or susceptibility to the development of fungal disease symptoms in Rosaideae, especially of Botrytis in cut roses (genus Rosa),
(2) the determination of cold tolerance in tree seedlings and the stage at which tree seedlings are cold-hardened sufficiently to be lifted,
(3) the determination of the ripening stage of fruit, especially pears after harvest,
(4) the determination of sensory stages or the deterioration (sensory decay) of fruit, especially apples after harvest,
(5) the determination of quality stage (degree of browning) and especially prediction of brown discoloration in edible mushrooms, especially harvested basidiomycetes such as Agaricus species, and
(6) the determination and prediction of firmness development (in particular of loss of firmness during post-harvest storage) in Solanaceous fruits, especially tomatoes (genus Solanum).

In its broadest term, the method for determining the quality stage and/or predicting quality traits according to the invention comprises:
(a) providing a nucleic acid sample (comprising RNA or corresponding cDNA) of a plant or plant part, or a plurality of plants (batch), or of an edible mushroom (or batch),
(b) analyzing the sample by determining the level of a set of indicator mRNA transcripts in the sample, which are indicative of the present stage and/or of the future status of a quality trait of the plant or plant part, or the batch of plants, or mushroom(s) or mushroom batch, and optionally
(c) identifying and selecting the plant (or mushroom) or the batch, which comprises a certain level of the indicator mRNA transcripts (i.e., a certain relative or absolute amount of mRNA or corresponding cDNA) for further use.

Optionally a step (b′) is inserted between steps (b) and (c), or replaces step (c), whereby said step comprises (b′) feeding the result obtained in (b) into a quality determination model or quality dynamics model that builds on a database of previously analyzed samples and that positions the sample at hand in the quality spectrum of interest and translating the outcome of the model into a practical advice for stakeholders in agro-production and agro-distribution, such as step (c) above.

In one embodiment steps (a) and (b) are repeated at regular time intervals, until the mRNA transcript levels are such that the plants, plant parts or batch is at a certain quality stage for step (b′) or (c).

For simplicity herein below reference to plants and plant parts or plant batches is understood to apply equally to edible mushrooms or mushroom parts or mushroom batches. In certain embodiments the plants or plant parts (or batches) are harvested parts, such as severed plant parts (e.g., cut flowers), harvested fruit (e.g., apples, pears, strawberries, etc.). The quality stage of the harvested product is determined once or more times. A harvested product may also refer to harvested plants or plant parts which have been further processed, such as sliced, diced, etc. and packaged into batches, but which are preferably still regarded as “fresh” (as defined above). In other embodiments, the quality stage is determined for the living, developing plant, such as plant seedlings.

In step (a), the plant or mushroom tissue, from which the nucleic acid sample is to be obtained, is collected and optionally processed and/or stored. Many methods for extracting nucleic acids from plant or mushroom material are known in the art. In a preferred embodiment, a tissue homogenate is made and some of the homogenate is placed onto FTA cards (Whatman FTA® Technology). The FTA cards capture the nucleic acids present in the homogenate and contain agents which protect the nucleic acids from degradation and damage. The homogenate on the FTA cards is allowed to dry (e.g., at least one hour, preferably at room temperature and preferably without assisting the drying period by heating or other means). The FTA cards comprising the tissue homogenate can then be stored (preferably in a desiccated environment) until the nucleic acids are captured therefrom for downstream processing.

In one embodiment steps (a) and (b) of the method are carried out at regular time intervals (e.g., once a week, once every two weeks, once a month, etc), so that a change in the level of mRNA transcripts (or the corresponding cDNAs) of the indicator genes can be determined, relative to the earlier level of transcripts. The relative change in mRNA transcript abundance (up-regulation down-regulation, no change in mRNA levels) is then used to select plants, plant parts or batches in step (c) and/or the change in transcript abundance is entered into the model in step (b′). Alternatively, the relative or absolute mRNA level of the indicator genes is compared to the level of the indicator RNAs in a suitable control sample. Such a control may for example consist of one or more nucleic acid samples of known quality stages (e.g., training batches or batches obtained at earlier time points) so that the indicator mRNA abundance is compared relative to that of the control sample. It is understood that the control expression data does not need to be produced at the same time as the sample data, but can have been produced previously, such as one or more training batches.

The nucleic acid sample of step (a) may be provided for several individual plants (or parts), or preferably for batches of several plants (or parts). cDNA samples of batches may be made by either first pooling tissue from several individuals and then obtaining the nucleic acid from the pooled tissue sample or by directly pooling the nucleic acid obtained from individual plants. Preferably, the nucleic acid sample in step (a) comprises or consists of total RNA, total mRNA or total cDNA. For example, the total mRNA is isolated (e.g., using polyA⁺ selection) and is used to make corresponding cDNA by reverse transcription.

The mRNA level (or corresponding cDNA level) of a set of defined indicator genes can be detected and quantified using various methods generally known in the art, such as (but not limited to) quantitative PCR methods, preferably quantitative RT-PCR, or nucleic acid hybridization based methods (for example microarray hybridization). Quantitative PCR (qPCR) may be carried out by conventional techniques and equipment, well known to the skilled person, described for instance in S. A. Bustin (Ed.), et al., A-Z of Quantitative PCR, IUL Biotechnology Series, #5, 2005. Preferably, labeled primers or oligonucleotides are used to quantify the amount of reaction product. Other techniques capable of quantifying relative and absolute amounts of mRNA in a sample, such as NASBA (Nucleic Acid Sequence Based Amplification), may also be suitably applied. A convenient system for quantification is the immunolabeling of the primers, followed by an immuno-lateral flow system (NALFIA) on a pre-made strip (Kozwich et al., 2000, Appl Envir Microbiol 66:2711-17; Koets et al., 2003, In: Proc Euro Food Chem XII—Strategies for Safe Food, September 2003, Brugge, Belgium, pp. 121-124; van Amerongen et al., 2005 In: Rapid methods for biological and chemical contaminants in food and feed. Eds. A. van Amerongen et al., Wageningen Academic Publishers, Wageningen, Netherlands, pages 105-126).

As a positive control for the RNA isolation, reverse transcriptase reaction, amplification reaction and detection step, amplification and detection of a constitutively expressed housekeeping gene may be included in the assay, such as ribosomal (18S or 25S) rRNAs, actin, tubulin or GAPDH. Primers may be labeled with direct labels such as FITC (fluorescein), Texas Red, Rhodamine and others or with tags such as biotin, lexA or digoxigenin which may be visualized by a secondary reaction with a labeled streptavidin molecule (for instance with carbon or a fluorescent label) or a labeled antibody (labeled with fluorescent molecules, enzymes, carbon, heavy metals, radioactive isotopes or with any other label).

In another embodiment, comparative hybridization is performed on mRNA or cDNA populations obtained from a plant or sample thereof, to a set of indicator gene sequences, which may optionally be tagged or labeled for detection purposes, or may be attached to a solid carrier such as a DNA array or microarray. Suitable methods for microarray detection and quantification are well described in the art and may for instance be found in: Applications of DNA Microarrays in Biology. R. B. Stoughton (2005) Annu Rev Biochem 74:53-82, or in Bowtell and Sambrook, DNA Microarrays: A Molecular Cloning Manual, Cold Spring Harbor Laboratory Press, 2003, pp. 625-7. To construct a DNA microarray, nucleic acid molecules (e.g., single stranded oligonucleotides according to the invention) are attached to a solid support at known locations or “addresses”. The arrayed nucleic acid molecules are complementary to the indicator nucleotide sequences according to the invention, and the location of each nucleic acid on the chip is known. Such DNA chips or microarrays, have been generally described in the art, for example, U.S. Pat. No. 5,143,854, U.S. Pat. No. 5,445,934, U.S. Pat. No. 5,744,305, U.S. Pat. No. 5,677,195, U.S. Pat. No. 6,040,193, U.S. Pat. No. 5,424,186, U.S. Pat. No. 6,329,143, and U.S. Pat. No. 6,309,831 and Fodor et al. (1991) Science 251:767-77, each incorporated by reference. See also technology providers, such as Affymetrix Inc. (www.affymetrix.com). These arrays may, for example, be produced using mechanical synthesis methods or light-directed synthesis methods that incorporate a combination of photolithographic methods and solid phase synthesis methods. Also methods for generating □abelled polynucleotides and for hybridizing them to DNA microarrays are well known in the art. See, for example, US 2002/0144307 and Ausubel et al., eds. (1994) Current Protocols in Molecular Biology, Current Protocols, John Wiley & Sons, Inc., New York; 1994 Supplement).

Herein, for each quality trait, a specific “set of indicator genes” is provided, whose expression level correlates with and is indicative of the present and/or future quality stage of the plant, plant part or batch. A “set of indicator genes” refers, therefore, to a defined number of genes whose expression level (mRNA abundance, or corresponding cDNA abundance) is being determined. A distinction can be made between the “main set”, which refers to a larger number of defined genes, and “sub-sets”, which refer to smaller numbers selected from the main set. Thus, either the main set of indicator transcripts may be detected or, preferably, a subset is detected. For example, the upregulation of one indicator mRNA transcript and the down regulation of another indicator mRNA transcript may already be sufficient to determine the quality of the plant or plant part (or batch). Thus, although in some Examples hereinbelow up to 30 indicator genes (and indicator transcripts) are provided, any subset thereof, such as 20, 10, 5, 4, 3, or 2 may already be sufficient. It is clear, that the robustness of the method is inversely related to the number of indicator transcripts being detected.

When referring to “indicator genes,” not only the specific nucleic acid sequences (mRNA or cDNA) of those genes (as depicted in the Sequence Listing) are referred to, but also “variants” of these sequences and fragments of the indicator genes or of the variants. A “variant” refers herein to a nucleic acid sequence which are “essentially identical” to the indicator genes provided, i.e., they comprises at least about 70, 75, 80, 85, 90, 95 98, 99% or more, nucleic acid sequence identity to the sequences provided herein (determined using pairwise alignment with the Needleman and Wunsch algorithm, as defined).

As mentioned, also fragments (e.g., oligonucleotides) of indicator genes (or of the variants of indicator genes) are encompassed and may be detected, or may be used for detection of the indicator transcript in a sample or batch. Fragments comprise any contiguous stretch of at least 8, 10, 12, 14, 15, 18, 20, 22, 25, 30, 40, 50, 100, 200, 500, 800, 900, 1000 or more nucleotides of an indicator gene or a variant thereof. Such fragments may be used as PCR primers or probes for detecting indicator genes by selectively hybridizing to the indicator mRNA or cDNA.

Variants may be isolated from natural sources, using for example stringent hybridization conditions or can be easily generated using methods known in the art, such as but not limited to nucleotide substitutions or deletions, de novo chemical synthesis of nucleic acid molecules or mutagenesis- or gene-shuffling techniques, etc.

Also provided are kits for carrying out the methods and nucleic acid carriers comprising sets of indicator genes, and/or variants and/or fragments of indicator genes, e.g., oligonucleotides of the indicator genes or of variants thereof. The kits may optionally also contain material and instructions for tissue/batch sampling, such as FTA cards and instructions for use or FTA cards onto which tissue homogenates have already been applied. Obviously, other material for sampling include other carriers for sample material (e.g., containers such as Eppendorf tubes or microtiter plates) and reagents, such as solvents, buffers, etc.

Nucleic acid carriers may for example be arrays and microarrays or DNA chips, comprising nucleotides on a glass, plastics, nitrocellulose or nylon sheets, silicon or any other solid surface, which are well known in the art and for instance described in Bowtell and Sambrook, 2003 (supra) and in Ausubel et al., Current protocols in Molecular Biology, Wiley Interscience, 2004. A carrier according to the current invention comprises at least two (or more, such as at least 3, 4, 5, 10, 15, 20, 25, 30, or more) (oligo-)nucleotide probes capable of selectively hybridizing with two indicator genes (mRNA or cDNA) present in a sample.

A kit for determining and/or predicting the quality stage of a sample comprises elements for use in the methods of the invention. Such a kit may comprise a carrier to receive therein one or more containers, such as tubes or vials. The kit may further comprise unlabeled or labeled (oligo)nucleotide sequences of the invention, e.g., to be used as primers, probes, which may be contained in one or more of the containers, or present on a carrier. The (oligo)nucleotides may be present in lyophilized form, or in an appropriate buffer. One or more enzymes or reagents for use in isolation of nucleic acids, purification, restriction, ligation and/or amplification reactions may be contained in one or more of the containers. The enzymes or reagents may be present alone or in admixture, and in lyophilized form or in appropriate buffers. The kit may also contain any other component necessary for carrying out the present invention, such as manuals, buffers, enzymes (such as preferably reverse transcriptase and a thermostable polymerase), pipettes, plates, nucleic acids (preferably labeled probes), nucleoside triphosphates, filter paper, gel materials, transfer materials, electrophoresis materials and visualization materials (preferably dyes, labeled antibodies or -enzymes) autoradiography supplies. Such other components for the kits of the invention are known per se. The kit may also comprise tissue samples and/or nucleic acid samples, such as suitable control samples.

### Assays and Kits for the Determination/Prediction of Botrytis Incidence or Susceptibility to Botrytis Infection in Rosoideae

Harvested flowers can suffer from Botrytis disease (grey mold, especially Botrytis cinerea) during its vase-life/post-harvest. Batches of roses can vary enormously in the percentage of flowers that show disease symptoms during vase-life. Using a standard visual screening method, it has been shown that there is no clear correlation between observed disease spots directly after harvest and the percentage infected flowers after a post-harvest chain simulation. It is a major problem for roses grown in African countries because transportation is expensive. Part of the biological variation in susceptibility is genetic, some cultivars are less susceptible to the fungus than others. However, non-genetic or phenotypic variation is of equal or even larger importance. Within-cultivar variation, e.g., caused by different growing or post-harvest conditions, is the main reason for quality miss-estimations.

So far no tests were available for determining the likelihood that roses will develop Botrytis symptoms after harvest. A visually screen does not give a conclusive prediction of Botrytis incidence. Detection of the pathogen itself is not sufficient because almost all roses contain spores of the fungus, but whether or not this will result in serious infection/disease symptoms is determined by several parameters, such as humidity, spore dose, temperature and, most of all, susceptibility (or sensitivity) of the plant to Botrytis infection and development caused by genetic differences (cultivar) and growth conditions.

Herein a method is provided which uses a set of 36 indicator genes to predict the susceptibility/resistance of Rosideae, especially roses, to Botrytis. Based on the expression level of the indicator genes conclusions can be drawn about the predicted quality class of a batch of roses (i.e., the predicted severity of Botrytis symptom development in the batch). Botrytis symptom development is assessed after 7 days at 21° C., 60% relative humidity, and under a light regime of 10 h light/14 h dark. The quality class labeled ‘good’ refers to batches of flowers with less then 10% of the flowers showing disease symptoms after this 7 days vase-life. Quality class labeled ‘moderate’ is used for batches having between 10-30% of the flowers showing Botrytis disease symptoms. Batches labeled as ‘bad’ refer to batches of which 30% or more of the flowers show disease symptoms. The expression level of the indicator genes can be used to predict in which class a ‘test’ batch falls.

In one aspect of the invention a method is provided for detecting the susceptibility/resistance of Rosideae (preferably Rosa, especially Rosa hybrida) to Botrytis infection and to the predicted development of Botrytis disease symptoms. The mRNA levels of the indicator genes, thus, serve as an indicator of the quality of the plants/plant tissue with respect to Botrytis resistance/susceptibility and thus with respect to the predicted severity of Botrytis symptoms after 7 days vase-life.

In one embodiment a method for determining the Botrytis susceptibility of plants or plant tissue of the family Rosideae, especially of the genera Rosa, Rubus and Fragaria, is provided.

The method provided herein uses a set of 36 indicator genes whose expression profile can be used as measurement of the likelihood that the plant tissue will develop no, mild, or severe Botrytis symptoms (i.e., belongs to the quality class labeled as good, moderate or bad, as described above). Based on the relative or absolute expression level of the described indicator genes conclusions can be drawn about the quality of plants or plant parts regarding Botrytis disease susceptibility/resistance.

As shown in the Examples, comparison of expression levels of a set of 36 genes in various batches of roses provided an indication of the susceptibility of a plant or batch to Botrytis infection and development subsequently during 1 week, under conditions similar to indoor vase-life. Thus, discrimination between batches which are of bad quality (susceptible and likely to develop severe Botrytis symptoms) and good quality (resistant and likely to develop no Botrytis symptoms) and moderate quality is possible.

The method for determining the Botrytis susceptibility of plants or plant parts (especially cut flowers) of the family Rosaideae, or in other words, for predicting the severity of Bortytis symptoms that will develop subsequently, comprises the following steps:
(a) providing a nucleic acid sample (comprising mRNA or cDNA) of a plant tissue (or a plurality of plant tissues; batch or batches),
(b) analyzing the sample by determining the level of a set of indicator mRNA transcripts in the sample, which are indicative of the Botrytis susceptibility/resistance of the plant or batch(es), and optionally
(c) identifying and selecting the plant or plant parts or batch(es) which comprises a certain level of the indicator mRNA transcripts, relative to suitable controls, for further use, e.g., good quality batches can be transported or sold, while bad quality batches can be destroyed.

Optionally, between step (b) and (c), or replacing step (c), the following step may be present:
(b′) feeding the resulting data obtained in (b) into a ripening model that relates expression of the indicator genes to ripening stage.
   This can be done using computer programs. An analogous step (b′) can be applied in any of the other embodiments of the invention.

Thus, plants or plant parts which comprise an “indicator mRNA profile” which is indicative of the Botrytis susceptibility/resistance level (i.e., the predicted severity of Botrytis symptoms which will develop) can be differentiated and handled differently.

Preferably, the method is carried out once (or several times, e.g., at regular time intervals, such as once every two days, once a week, etc.) after harvest, in order to sort plants or batches into different groups based on prediction of Botrytis susceptibility.

Any tissue of the plant may be used in the method, for example leaf, flower, stem, root, twigs, fruit, seeds, embryos, pollen, whole seedlings, etc., although preferably, the petals of the flowers are used to prepare the nucleic acid sample. For roses, especially the outer petals are preferred. To have a good coverage of the potency of the whole batch, 20 outer petals are preferably sampled randomly from the batch. Definition of a batch is a product, sampled on the same day from the same greenhouse that have been treated the same from harvest until sampling. Thus, first suitable tissue is sampled for nucleic acid extraction. In the present method, it is preferred that in step (a) nucleic acid samples are prepared by harvesting petal samples of a plant, grinding and mixing sample material and extracting the total RNA or total mRNA from the sample. The sample can be prepared using known nucleic acid extraction methods, e.g., total RNA or mRNA purification methods and kits provided in the art (e.g., RNAeasy kits of Qiagen, kits of SIGMA, Clonetech, etc.). The mRNA may be reverse transcribed into cDNA, using known methods. Expression levels of the genes are preferably analyzed relative to the levels of a training set of batches (same materials and same cultivar) with known occurrence of Botrytis infection in the flowers in a vase-life test. Using a training set of at least about 30, more preferably at least about 45 samples, preferably at least about 10, more preferably at least about 15 from each of the three quality classes, the genes expression of new ‘test’ batches are studied relative to the gene expression of the training set batches in order to predict in which quality class the new ‘test’ batches fit best.

In step (b), the nucleic acid sample is analyzed for the presence and the level (abundance or relative level) of indicator RNA transcripts (mRNA) in the sample. When referring to indicator RNA in a sample, it is clear that this also encompasses indicator cDNA obtainable from said mRNA. Preferably Real time RT-PCR using primers which amplify the indicator transcripts (or a subset thereof) is used as described in the Examples.

In one embodiment, the mRNA (or cDNA) sequences, which are detected in a sample, and which are indicative of the Botrytis susceptibility/resistance of the tissue are SEQ ID NO: 77-109 and/or SEQ ID NO: 172-174, or variants thereof, or fragments of any of these (the main set of indicator genes). Thus, any method may be used to detect the relative or absolute amounts of SEQ ID NO: 77-109 and/or SEQ ID NO: 172-174, variants of SEQ ID NO: 77-109 and/or SEQ ID NO: 172-174, or fragments of these in the sample(s). For example, PCR primer pairs which amplify fragments of each of SEQ ID NO: 77-109 and/or SEQ ID NO: 172-174 may be used in quantitative RT-PCR reactions. Alternatively, the nucleic acid sample may be labeled and hybridized to a nucleic acid carrier comprising oligonucleotides of each of SEQ ID NO: 77-109 and/or SEQ ID NO: 172-174 (and/or variants thereof), whereby the level of these transcripts in the sample is determined. Expression levels may be normalized against housekeeping gene expression levels, such as those of SEQ ID NO: 110-112.

In another embodiment a subset of indicator genes is detected in the sample, and the transcript level is compared to the transcript level of the same subset of indicator genes in a suitable control. A subset may comprise any subset of SEQ ID NO: 77-109 and/or SEQ ID NO: 172-174 (or variants thereof), such as the detection of 20, 15, 10 or less of the sequences.

The expression profile of SEQ ID NO: 77-109 and/or SEQ ID NO: 172-174, and/or variants thereof, predicts the severity of Botrytis symptoms which develop later on, after about one week at room temperature (about 21° C.). Thus, when the expression levels of the indicator sequences is analyzed and the expression of the indicator genes is such that it fits the expression levels of the batches of the training set labeled as ‘good’ (measured using exactly the same method, using the same protocol and software programs, such as e.g., Predicted Analysis of Microarray or PAM), it is very likely that the new tested plant material also will have relative low occurrence (less then 10%) of Botrytis diseased flowers during post-harvest vase-life, as was found for the batches in quality class ‘good’ of the train set.

When the expression levels of the indicator sequences is analyzed and the expression of the indicator genes is such that it fits the expression levels of the batches of the training set labeled as ‘moderate’ (measured using exactly the same method, using the same protocol and software programs, e.g., Predicted Analysis of Microarray), it is very likely that the new tested plant material also will have relative moderate occurrence (between 10-30%) of Botrytis diseased flowers during post-harvest vase-life, as was found for the batches in quality class ‘moderate’ of the train set.

When the expression levels of the indicator sequences is analyzed and the expression of the indicator genes is such that it fits the expression levels of the batches of the train set labeled as ‘bad’ (measured using exactly the same method, using the same protocol and software programs, such as e.g., Predicted Analysis of Microarray), it is very likely that the new tested plant material also will have relative high occurrence (more then 30%) of Botrytis diseased flowers during post-harvest vase-life, as was found for the batches in quality class ‘bad’ of the train set.

In a preferred embodiment the “minimal set” of indicator mRNAs comprises at least 2, 3, 4, 5, 6, 7, 8, 9, 10, 15, 20 or more mRNAs selected from SEQ ID NO: 77-109 and/or SEQ ID NO: 172-174 (or variants or fragments thereof).

As already mentioned, it is understood that also “variants” of SEQ ID NO: 77-109 and/or SEQ ID NO: 172-174 may be detected in a sample, such as nucleic acid sequences essentially similar to any of SEQ ID NO: 77-109 and/or SEQ ID NO: 172-174, i.e., comprising at least 70, 75, 80, 85, 90, 95, 98, 99% or more nucleic acid sequence identity to any of SEQ ID NO: 77-109 and/or SEQ ID NO: 172-174. Such variants may for example be present in different species or different varieties.

The actual method used for determining the level of the set of indicator mRNA transcripts is not important. Any gene expression profiling method may be used, such as RT-PCR, microarrays or chips, Northern blot analysis, cDNA-AFLP, etc. See elsewhere herein. For example, PCR primer pairs specific for each of SEQ ID NO: 77-109 and/or SEQ ID NO: 172-174 (or variants thereof) may be designed using known methods. Alternatively, nucleic acid probes, which hybridize to SEQ ID NO: 77-109 and/or SEQ ID NO: 172-174 (or variants thereof) may be made for use in the detection. Any fragment of at least about 10, 12, 14, 15, 20, 22, 30, 50, 100, 200, 300, 500 or more consecutive nucleotides of SEQ ID NO: 77-109 and/or SEQ ID NO: 172-174, or the complement strand, or of a variant of SEQ ID NO: 77-109 and/or SEQ ID NO: 172-174, may be suitable for detection of the full length transcript in a sample. Equally, any fragment of a “variant” of any one of SEQ ID NO: 77-109 and/or SEQ ID NO: 172-174 (as defined above) may be used.

In one embodiment a carrier is provided comprising nucleic acid molecules SEQ ID NO: 77-109 and/or SEQ ID NO: 172-174, variants of SEQ ID NO: 77-109 and/or SEQ ID NO: 172-174 and/or most preferably fragments (oligonucleotides) of any of these or of a subset of any of these. The carrier may, for example, be contacted under hybridizing conditions with the (labeled) nucleic acid sample of the sample of step (a), allowing detection of the level of each of the indicator transcripts present in the sample. The carrier may also comprise housekeeping nucleic acids, such as for example single stranded or double stranded oligonucleotides of SEQ ID NO: 110 to 112, or variants of these.

In a further embodiment, kits, oligonucleotides (e.g., PCR primers, nucleic acid probes) and antibodies are provided, for determining the Botrytis sensitivity/resistance of harvested plant tissue. Such kits comprise instructions for use and one or more reagents for use in the method. Optionally, tissue samples or nucleic acid samples suitable as controls may be included. Thus, such a kit may comprise a carrier to receive therein one or more containers, such as tubes or vials. The kit may further comprise unlabeled or labelled oligonucleotide sequences of the invention (SEQ ID NO: 77-109 and/or SEQ ID NO: 172-174, or variants thereof, or parts thereof, such as degenerate primers or probes and optionally also oligonucleotides of housekeeping genes, e.g., those of SEQ ID NO: 110-112 or others), e.g., to be used as primers, probes, which may be contained in one or more of the containers, or present on a carrier. The oligonucleotides may be present in lyophilized form, or in an appropriate buffer. One or more enzymes or reagents for use in isolation of nucleic acids, purification, restriction, ligation and/or amplification reactions may be contained in one or more of the containers. The enzymes or reagents may be present alone or in admixture, and in lyophilised form or in appropriate buffers. The kit may also contain any other component necessary for carrying out the present invention, such as manuals, buffers, enzymes (such as preferably reverse transcriptase and a thermostable polymerase), pipettes, plates, nucleic acids (preferably labelled probes), nucleoside triphosphates, filter paper, gel materials, transfer materials, electrophoresis materials and visualization materials (preferably dyes, labelled antibodies or -enzymes) autoradiography supplies.

### Assays and Kits for the Determination of Cold Tolerance in Tree Seedlings of the Family Fagaceae, Especially Beech Seedlings

In one embodiment of the invention a method for determining cold tolerance (or frost tolerance) of Fagaceae seedlings, especially in beech seedlings is provided.

Tree seedlings grown in nurseries have to be lifted and transferred to cold storage in autumn. However, lifting at a suboptimal moment, when the seedling is not yet fully cold-hardened, causes reduced vitality of the plants after storage. The only available test method for seedling hardiness is to date electrolyte leakage measurements. However, these measurements are not very accurate and are highly time consuming, taking at least 4 days. This is difficult to fit into nursery logistic schedules. In addition seedlings often have to be transferred to a test lab. During transport the physiology of the plant can be influenced.

The method provided herein uses a set of 29 indicator genes whose expression profile can be used as measurement for the cold tolerance level of Fagaceae seedlings, preferably beech seedlings. Based on the relative or absolute expression level of the described indicator genes conclusions can be drawn about the level of cold tolerance that is reached in tree seedlings. It was found that, as soon as the expression of the 29 cold tolerance related genes stabilizes, cold tolerance has reached the maximal level (see Examples).

The method for determining cold tolerance of Fagaceae seedlings comprises the following steps:
(a) providing a nucleic acid sample (comprising mRNA or cDNA) of a batch of Fagaceae seedlings (e.g., a representative sample of buds),
(b) analyzing the sample by determining the level of a set of indicator mRNA transcripts in the sample, which are indicative of the cold tolerance stage of the Fagaceae seedlings, and optionally
(c) identifying and selecting the Fagaceae seedlings which comprises a certain level of the indicator mRNA transcripts, relative to suitable controls, for further use, e.g., for transfer to cold storage. Thus, seedlings which comprise an “indicator mRNA profile” which is indicative of cold-tolerance are identified.

Optionally, between step (b) and (c), or replacing step (c), the following step may be present:
(b′) feeding the resulting data obtained in (b) into a ripening model that relates expression of the indicator genes to ripening stage.
   This can be done using computer programs. An analogous step (b′) can be applied in any of the other embodiments of the invention.

The method can be applied to any tree seedlings of the family “Fagaceae”, including for example varieties of Fagus sylvatica L., other species from the genus Fagus, such as Fagus crenata (Japanese Beech), Fagus engleriana (Chinese Beech), Fagus grandifolia (American Beech), Fagus hayatae (Taiwan Beech), Fagus japonica (Japanese Blue Beech), Fagus longipetiolata (South Chinese Beech), Fagus lucida (Shining Beech), Fagus □abelled (Mexican Beech or Haya), Fagus orientalis (Oriental Beech), and other genera of the family Fagaceae, such as Castanea (chestnuts) and Quercus (oaks) species. In a preferred embodiment seedlings of the genus Fagus, more preferably of the species Fagus sylvatica are used. The seedlings may be of various ages, e.g., one or two years old. They may have been grown in the field or in a controlled environment. Preferably, nucleic acids of a batch of seedlings refers to nucleic acids obtained from a batch of seedlings grown at the same location and under the same growth conditions.

The method can be used to identify and select those tree seedlings which are ready to be transferred to cold storage, without reducing the viability of the seedlings during or after cold storage. Cold storage refers to storage of seedlings for several weeks or months in controlled environments at temperatures of −2 to +4° C. Thus, the optimal developmental stage of the plants for transfer into cold storage can be assessed. Preferably, the RNA profile of the indicator genes, or of a subset thereof, is analyzed more then once, i.e., at one or more time intervals. This allows the expression level of the indicator genes to be compared relative to the earlier level(s). For example, once a month, once every 3, 2, or 1 week, the mRNA profiling method may be repeated until the mRNA profile is found which indicates that the plants are now cold-hardened and ready to be transferred to cold storage. Alternatively, expression levels of a test batch are compared to the expression levels of one or more training batches (for example a batch of cold-sensitive seedlings).

Any tissue of the plant may be used in the method, for example leaf, flower, stem, root, twigs, fruit, seeds, embryos, pollen, whole seedlings, etc., although preferably, the buds of the tree seedlings are used to prepare the nucleic acid sample. Most preferably apical buds are used. Thus, first suitable tissue is sampled for nucleic acid extraction. In the present method, it is preferred that in step (a) a nucleic acid sample is prepared by harvesting bud-tissue of a representative number of plants and extracting the total RNA or total mRNA from the pooled sample. The sample can be prepared using known nucleic acid extraction methods, e.g., total RNA or mRNA purification methods and kits provided in the art (e.g., RNAeasy kits of Qiagen, kits of BIORAD, Clontech, Dynal etc.). The mRNA may be reverse transcribed into cDNA, using known methods.

In step (b), the nucleic acid sample is analyzed for the presence and the level (abundance or relative level) of indicator RNA transcripts (mRNA) in the sample. When referring to indicator RNA in a sample, it is clear that this also encompasses indicator cDNA obtainable from said mRNA.

In one embodiment, the mRNA (or cDNA) sequences indicative of cold tolerance which are detected in the sample are SEQ ID NO: 1-29, or variants thereof, or fragments of any of these (the main set of indicator genes). Thus, any method may be used to detect the relative or absolute amounts of SEQ ID NO: 1-29, variants of SEQ ID NO: 1-29, or fragments of these in the sample(s). For example, PCR primer pairs which amplify fragments of each of SEQ ID NO: 1-29 may be used in quantitative RT-PCR reactions. Alternatively, the nucleic acid sample may be labeled and hybridized to a nucleic acid carrier comprising oligonucleotides of each of SEQ ID NO: 1-29, whereby the level of these transcripts in the sample is determined.

In another embodiment a subset of indicator genes is detected in the sample, and the transcript level is compared to the transcript level of the same subset in a suitable control.

SEQ ID NO: 1-15, and variants thereof, are upregulated in cold-tolerant seedlings compared to cold sensitive seedlings (referred to as “upregulated transcripts” indicative of cold tolerance). SEQ ID NO: 16-27, and variants thereof, are downregulated in cold tolerant seedlings compared to cold sensitive seedlings (referred to as “downregulated transcripts” indicative of cold tolerance). Further, SEQ ID NO: 28 and 29, and variants thereof, are about equal in their expression level in cold tolerant compared to cold sensitive seedling. Most preferably, the mRNA or cDNA level of a set of at least 2, 3, 4, 5, 6, 7, 8, 9, 10, 15, 20, 25 or more of any one of SEQ ID NO: 1-29, or variants or fragments thereof, is determined in the sample in step (b). The expression level of the indicator transcripts is preferably compared to the level of transcript of a suitable control, e.g., either the same plant analyzed at an earlier stage, or another suitable control sample, such as the sample of a cold-sensitive beech seedling.

In a preferred embodiment the expression level of at least one “upregulated transcript” and at least one “downregulated transcript” are detected. Optionally, also the expression level of a “constant” transcript, e.g., SEQ ID NO: 28 and/or 29, may be detected. Thus, the “minimal set” of indicator mRNAs comprises at least two mRNAs, one selected from SEQ ID NO: 1-15 (or variants or fragments thereof) and one selected from SEQ ID NO: 16-27 (or variants or fragments thereof).

As already mentioned, it is understood that also “variants” of SEQ ID NO: 1-29 may be detected in a sample, such as nucleic acid sequences essentially similar to any of SEQ ID NO: 1-29, i.e., comprising at least 70, 75, 80, 85, 90, 95, 98, 99% or more nucleic acid sequence identity to any of SEQ ID NO: 1-29. Such variants may for example be present in different tree species or different varieties.

The actual method used for determining the level of the set of indicator mRNA transcripts is not important. Any gene expression profiling method may be used, such as RT-PCR, microarrays or chips, Northern blot analysis, cDNA-AFLP, etc. See elsewhere herein. For example, PCR primer pairs for each of SEQ ID NO: 1-29 may be designed using known methods. Suitable primer pairs are, for example, the PCR primer pairs provided in the Examples and depicted in SEQ ID NO: 30-41. In one embodiment of the invention two or more of these primer pairs are used in the method. Alternatively, nucleic acid probes, which hybridize to SEQ ID NO: 1-29 may be made for use in the detection. Any fragment of 15, 20, 22, 30, 50, 100, 200, 300, 500 or more consecutive nucleotides of SEQ ID NO: 1-29, or the complement strand, or of a variant of SEQ ID NO: 1-29, may be suitable for detection of the full length transcript in a sample. Equally, any fragment of a “variant” of any one of SEQ ID NO: 1-29 (as defined above) may be used.

In one embodiment a carrier is provided comprising nucleic acid molecules SEQ ID NO: 1-29, variants of SEQ ID NO: 1-29 and/or most preferably fragments (oligonucleotides) of any of these or of a subset of any of these. The carrier may, for example, be contacted under hybridizing conditions with the (labeled) nucleic acid sample of the sample of step (a), allowing detection of the level of each of the indicator transcripts present in the sample.

If the expression profile of the indicator mRNAs of the seedling corresponds to the profile of cold-tolerant tree seedlings, the plant can be identified and selected for further use. Preferably, the seedlings can be transferred to cold storage, as this is now safe to do (without risking reduced viability). Therefore, the method provides a way of determining whether or not seedlings can be transferred to cold storage without loss of viability during storage.

In a further embodiment, kits, oligonucleotides (e.g., PCR primers, nucleic acid probes) and antibodies are provided, for determining the cold-tolerance of tree seedlings. Such kits comprise instructions for use and one or more reagents for use in the method. Optionally, tissue samples or nucleic acid samples suitable as controls may be included. Thus, such a kit may comprise a carrier to receive therein one or more containers, such as tubes or vials. The kit may further comprise unlabeled or □abelled oligonucleotide sequences of the invention (SEQ ID NO: 1-29, or variants thereof, or parts thereof, such as degenerate primers or probes), e.g., to be used as primers, probes, which may be contained in one or more of the containers, or present on a carrier. The oligonucleotides may be present in lyophilized form, or in an appropriate buffer. One or more enzymes or reagents for use in isolation of nucleic acids, purification, restriction, ligation and/or amplification reactions may be contained in one or more of the containers. The enzymes or reagents may be present alone or in admixture, and in lyophilised form or in appropriate buffers. The kit may also contain any other component necessary for carrying out the present invention, such as manuals, buffers, enzymes (such as preferably reverse transcriptase and a thermostable polymerase), pipettes, plates, nucleic acids (preferably labelled probes), nucleoside triphosphates, filter paper, gel materials, transfer materials, electrophoresis materials and visualization materials (preferably dyes, labelled antibodies or -enzymes) autoradiography supplies.

### Assays and Kits for the Determination of the Ripening Stage of Fruit of the Family Maloideae, Especially Pears

Harvested fruit, such as pears, are often stored for several months in cold storage before they are transferred to retail. Storage disorders occur regularly and are usually related to developmental stage at the time of harvest. A proper monitoring of the ripening process would allow selecting batches that are likely to maintain high quality during storage and would prevent the economic losses associated with storage disorders.

At present no reliable measurement for discriminating between various stages of ripening of fruit, such as pears, is available. Firmness measurements are sometimes used, but they have proven to lack the accuracy, that is needed for a good indicator of developmental stage.

In one embodiment a method for determining the ripening stage of fruit of the family Maloideae, especially of the genus Pyrus or Malus, is provided.

The method provided herein uses a set of at least 2, 3, 4, 5 or more indicator genes whose expression profile can be used to discriminate between different (relative) ripening stages of fruit of the family Maloideae, preferably pear. Based on the relative or absolute expression level of the described indicator genes conclusions can be drawn about the ripening stage of the fruit that is reached when the fruit are still attached to the plant or post-harvest.

As shown in the Examples, comparison of expression levels of a set of genes in various batches of pears provided information about relative ripening stages. The present method is much more informative than firmness measurements (see Examples). Thus, discrimination between batches is possible in cases where firmness measurements fail.

The method for determining the ripening stage of fruit of the family Maloideae comprises the following steps:
(a) providing a nucleic acid sample (comprising mRNA or cDNA) from fruit or fruit tissue (or a plurality of fruit or fruit tissues; batch),
(b) analyzing the sample by determining the level of a set of indicator mRNA transcripts in the sample, which are indicative of the ripening stage of the fruit, and optionally
(c) identifying and selecting the fruit which comprises a certain level of the indicator mRNA transcripts, relative to suitable controls, for further use, e.g., for harvest and/or for (cold)storage, processing or sale.

Thus, fruit which comprise an “indicator mRNA profile” which is indicative that the fruit is at a ripening stage which allows harvest and/or cold-storage of the fruit, without quality loss during cold storage, are identified. Also the ripening stage during storage can be followed using the method, allowing the discrimination between batches, which are at different ripening stages. Similarly, storage conditions can be optimized, by testing the effect of various parameters (temperature, humidity, etc.) on the ripening process of fruit.

The method is especially suitable for relative discrimination between batches from the same season. With this method it is possible to discriminate between batches, in situations where known methods (such as firmness measurement) fail. As absolute expression levels will vary from season to season, training batches are preferably developed each season for different ripening stages. In these training batches the ripening stage is roughly correlated to the expression level of the indicator genes. The indicator mRNA level in a “test” batch is then compared relative to that of the training batches and can thereby be assigned a ripening stage. Thereby, relatively more ripe and/or relatively less ripe batches can be differentiated and optionally selected for further use.

The expression level of several indicator genes (SEQ ID NO: 43-46 and/or SEQ ID NO: 158-161 and/or SEQ ID NO: 163-165, and/or SEQ ID NO: 167-171 and variants thereof) increases progressively with ripening, while three genes (SEQ ID NO: 42, SEQ ID NO: 162 and SEQ ID NO: 166 and variants thereof) remain constant. Thus, a relative higher expression level of any of SEQ ID NO: 43-46 and/or SEQ ID NO: 158-161 and/or SEQ ID NO: 163-165, and/or SEQ ID NO: 167-171 (and/or variants thereof) in a batch (e.g., at least about 5×, 10×, 20×, 50×, 100× higher mRNA levels) indicates a more advanced ripening stage of the batch.

The method can be applied to determine the ripening stage of fruit of the family Maloideae. In a preferred embodiment fruit of the genus Pyrus or Malus, preferably of the species Pyrus communis L. (e.g., cv. Conference), but may also be applied on any other cultivar of the species, or in other genera from the subfamily of Maloideae.

The method can be used to identify and select those fruit which are ready to be harvested and transferred to cold storage, without reducing the quality during or after cold storage. Cold storage refers to storage of seedlings for several weeks or months in controlled environments at temperatures of −2 to +4° C. Thus, the optimal developmental stage of the plants for harvest and/or transfer into cold storage can be assessed. Alternatively, the ripening stages of different batches, e.g., already in storage, can be compared and unripe or ripe batches can be selected for further use.

Preferably, the RNA profile of the indicator genes, or of a subset thereof, is analyzed more then once, i.e., at one or more time intervals. This allows the expression level of the indicator genes to be compared relative to the earlier level(s). The ripening progress of a batch can thereby be followed over time, either prior to harvest and/or after harvest. For example, once a month, once every 3, 2, or 1 week, or once every few days (e.g., at 2 day, 3 day, 4 day or 5 day intervals) the mRNA profiling method may be repeated until the mRNA profile is found which indicates that the fruit are now ready to be harvested and/or ready to be transferred to cold storage.

Any tissue of the plant may be used in the method, for example leaf, flower, stem, root, twigs, fruit, seeds, embryos, pollen, whole seedlings, etc., although preferably, the mesocarp tissue of fruit is used to prepare the nucleic acid sample. Most preferably the control tissue is taken from unripe fruit well before harvest and the expression level of the indicator genes may be compared relative to the level in this unripe batch. For example, the unripe fruit or batch may have an average firmness of at least 6 Newton (measured by penetrometer analysis). Thus, first suitable tissue is sampled for nucleic acid extraction. In the present method, it is preferred that in step (a) nucleic acid samples are prepared by harvesting fruit samples of a plant and extracting the total RNA or total mRNA from the sample. The sample can be prepared using known nucleic acid extraction methods, e.g., total RNA or mRNA purification methods and kits provided in the art (e.g., RNAeasy kits of Qiagen, kits of BIORAD, Clonetech, Dynal etc.). The mRNA may be reverse transcribed into cDNA, using known methods.

In step (b), the nucleic acid sample is analyzed for the presence and the level (abundance or relative level) of indicator RNA transcripts (mRNA) in the sample. When referring to indicator RNA in a sample, it is clear that this also encompasses indicator cDNA obtainable from said mRNA.

In one embodiment, the mRNA (or cDNA) sequences indicative of the fruit ripening stage detected in the sample are SEQ ID NO: 42-46 and/or SEQ ID NO: 158-171, or variants thereof, or fragments of any of these. Thus, any method may be used to detect the relative or absolute amounts of one or more of SEQ ID NO: 42-46 and/or SEQ ID NO: 158-171, variants of SEQ ID NO: 42-46 and/or SEQ ID NO: 158-171, or fragments of these in the sample(s). For example, PCR primer pairs which amplify fragments of each of SEQ ID NO: 42-46 and/or SEQ ID NO: 158-171 may be used in quantitative RT-PCR reactions. Alternatively, the nucleic acid sample may be labeled and hybridized to a nucleic acid carrier comprising oligonucleotides of each of SEQ ID NO: 42-46 and/or SEQ ID NO: 158-171, whereby the level of these transcripts in the sample is determined.

SEQ ID NO: 43-46, SEQ ID NO: 158-161, SEQ ID NO: 163-165 and SEQ ID NO: 167-171, and variants thereof, are upregulated during fruit ripening compared to unripe fruit (referred to as “upregulated transcripts” indicative of fruit ripening). Further, SEQ ID NO: 42, SEQ ID NO: 162 and SEQ ID NO: 166, and variants thereof, are about equal in their expression level in ripe fruit tissue compared to unripe tissue (referred to as “constant transcript” indicative of fruit ripening). Most preferably, the mRNA or cDNA level of a set of at least 2, 3, 4 or 5 of any one of SEQ ID NO: 42-46 and/or SEQ ID NO: 158-171, or variants or fragments thereof, is determined in the sample in step (b). The expression level of the indicator transcripts is preferably compared to the level of transcript of a suitable control, e.g., either the same fruit batch analyzed at an earlier stage, or another suitable control sample, such as the sample of an unripe fruit, and/or training batches.

As already mentioned, it is understood that also “variants” of SEQ ID NO: 42-46 and/or of SEQ ID NO: 158-171 may be detected in a sample, such as nucleic acid sequences essentially similar to any of SEQ ID NO: 42-46 and/or of SEQ ID NO: 158-171, i.e., comprising at least 70, 75, 80, 85, 90, 95, 98, 99% or more nucleic acid sequence identity to any of SEQ ID NO: 42-46 and/or of SEQ ID NO: 158-171. Such variants may for example be present in different species or different varieties.

The actual method used for determining the level of the set of indicator mRNA transcripts is not important. Any gene expression profiling method may be used, such as RT-PCR, microarrays or chips, Northern blot analysis, cDNA-AFLP, etc. See elsewhere herein. For example, PCR primer pairs for each of SEQ ID NO: 42-46 and/or of SEQ ID NO: 158-171 may be designed using known methods. Suitable primer pairs are, for example, the PCR primer pairs provided in the Examples and depicted in SEQ ID NO: 47-56 and/or of SEQ ID NO: 158-171. In one embodiment of the invention two or more of these primer pairs are used in the method. Alternatively, nucleic acid probes, which hybridize to SEQ ID NO: 42-46 and/or of SEQ ID NO: 158-171 may be made for use in the detection. Any fragment of at least about 15, 20, 22, 30, 50, 100, 200, 300, 500 or more consecutive nucleotides of SEQ ID NO: 42-46 and/or of SEQ ID NO: 158-171, or the complement strand, or of a variant of SEQ ID NO: 42-46 and/or of SEQ ID NO: 158-171, may be suitable for detection of the full length transcript in a sample. Equally, any fragment of a “variant” of any one of SEQ ID NO: 42-46 and/or of SEQ ID NO: 158-171 (as defined above) may be used.

In one embodiment a carrier is provided comprising nucleic acid molecules SEQ ID NO: 42-46 and/or of SEQ ID NO: 158-171, variants of SEQ ID NO: 42-46 and/or of SEQ ID NO: 158-171 and/or most preferably fragments (oligonucleotides) of any of these or of a subset of any of these. The carrier may, for example, be contacted under hybridizing conditions with the (labeled) nucleic acid sample of the sample of step (a), allowing detection of the level of each of the indicator transcripts present in the sample.

In practice the expression profile of the indicator mRNAs of the fruit can be used determine the optimal moment for harvest, depending on choices for downstream chains, e.g., ready- to-eat delivery to local retail, export or long-term storage without risking storage disorders developing.

In a further embodiment, kits, oligonucleotides (e.g., PCR primers, nucleic acid probes) and antibodies are provided, for determining the ripening stage of fruit. Such kits comprise instructions for use and one or more reagents for use in the method. Optionally, tissue samples or nucleic acid samples suitable as controls may be included. Thus, such a kit may comprise a carrier to receive therein one or more containers, such as tubes or vials. The kit may further comprise unlabeled or labelled oligonucleotide sequences of the invention (SEQ ID NO: 42-46 and/or of SEQ ID NO: 158-171, or variants thereof, or parts thereof, such as degenerate primers or probes), e.g., to be used as primers, probes, which may be contained in one or more of the containers, or present on a carrier. The oligonucleotides may be present in lyophilized form, or in an appropriate buffer. One or more enzymes or reagents for use in isolation of nucleic acids, purification, restriction, ligation and/or amplification reactions may be contained in one or more of the containers. The enzymes or reagents may be present alone or in admixture, and in lyophilised form or in appropriate buffers. The kit may also contain any other component necessary for carrying out the present invention, such as manuals, buffers, enzymes (such as preferably reverse transcriptase and a thermostable polymerase), pipettes, plates, nucleic acids (preferably labelled probes), nucleoside triphosphates, filter paper, gel materials, transfer materials, electrophoresis materials and visualization materials (preferably dyes, labelled antibodies or -enzymes) autoradiography supplies.

### Assays and Kits for the Determination of Sensory Decay of Fruit of the Family Maloideae, Preferably of the Genus Malus or Pyrus, Especially Apples

Fruit, such as apples are stored for up to 9 months before they are transferred to retail and consumers. During storage quality decay may occur, of which the severity is related to the physiological status of the apples at the start of the storage period. A commonly occurring storage disorder is “mealiness”. This characteristic is cultivar (genotype) dependent but there are also large batch differences (phenotype). Soft apples are B quality and often have to be discarded.

Mealiness of apples is to date measured using a penetrometer, which registers firmness. In practice fruit samples are taken during storage and tested for sensory aspects by human taste, or using firmness measurements. All these measurements detect secondary effects and can not be used for early warning.

In one aspect of the invention a method is provided for detecting early changes in relative expression levels of indicator genes, which serve as an early warning for sensory decay in fruit, especially apples after harvest. Using method which rely on firmness (softening) or sensory analysis by humans (assessing mealiness, flavor, odor, juiciness, etc.) one can only detect deterioration of fruit quality once it is already quite advanced (e.g., three weeks after placement into suboptimal storage conditions). In the present method much earlier signs of quality loss, especially sensory quality loss, can be determined (already one week after placement into suboptimal storage conditions).

In one embodiment a method for detecting sensory decay in fruit of the family Maloideae, especially of the genus Pyrus or Malus, is provided.

The method provided herein uses a set of 20 indicator genes whose expression profile can be used as measurement for the (relative) sensory decay of fruit of the family Maloideae, preferably apple. Based on the relative or absolute expression level of the described indicator genes conclusions can be drawn about the stage of sensory decay of the fruit that is reached post-harvest.

As shown in the Examples, comparison of expression levels of a set of 20 genes (or variants thereof, or subsets thereof) in various batches of apples provided an early warning of sensory decay. Thus, discrimination between batches which are starting to develop sensory decay and between good quality batches is possible.

The method for detecting signs of sensory decay of fruit of the family Maloideae comprises the following steps:
(a) providing a nucleic acid sample (comprising mRNA or cDNA) of a fruit or fruit tissue (or a plurality of fruit or fruit tissues; batch),
(b) analyzing the sample by determining the level of a set of indicator mRNA transcripts in the sample, which are indicative of the sensory decay stage of the fruit, and optionally
(c) identifying and selecting the fruit which comprises a certain level of the indicator mRNA transcripts, relative to suitable controls (e.g., training batches, or batches of known sensory decay stages such as a sample taken at harvest time when no decay has taken place yet), for further use, e.g., for removal from storage and immediate processing or sale.

Thus, fruit which comprise an “indicator mRNA profile” which is indicative that the fruit (or batch) has already initiated sensory decay allows decaying fruit or batches comprising decaying fruit to be differentiated and removed from storage. Also the sensory decay during storage can be followed using the method, allowing the discrimination between batches, which are at different sensory decay stages. Similarly, storage conditions can be optimized, by testing the effect of various parameters (temperature, humidity, etc.) on the sensory decay process of fruit.

The method can be applied to determine the sensory decay stage of fruit of the family Maloideae. In a preferred embodiment fruit of the genus Pyrus or Malus, preferably of the species Malus domestica (e.g., cv. Cox orange).

Preferably, the RNA profile of the indicator genes, or of a subset thereof, is analyzed more then once, i.e., at one or more time intervals. This allows the expression level of the indicator genes to be compared relative to the earlier level(s). For example, once a month, once every 3, 2, or 1 week, or several times a week, the mRNA profiling method may be repeated until the mRNA profile is found which indicates that the fruit or batch shows early signs of sensory decay.

Any tissue of the plant may be used in the method, for example leaf, flower, stem, root, twigs, fruit, seeds, embryos, pollen, whole seedlings, etc., although preferably, the mesocarp tissue of fruit is used to prepare the nucleic acid sample. Most preferably the control tissue is taken from fruit at harvest time, when no sensory decay has occurred. SEQ ID NO: 57-66 (and variants thereof) are upregulated, while SEQ ID NO: 67-76 (and variants thereof) are downregulated relative to a sample taken at harvest time, indicating sensory decay of the batch. Thus, first suitable tissue is sampled for nucleic acid extraction. In the present method, it is preferred that in step (a) nucleic acid samples are prepared by harvesting fruit samples of a plant and extracting the total RNA or total mRNA from a pooled sample. The sample can be prepared using known nucleic acid extraction methods, e.g., total RNA or mRNA purification methods and kits provided in the art (e.g., RNAeasy kits of Qiagen, kits of SIGMA, Clonetech, etc.). The mRNA may be reverse transcribed into cDNA, using known methods.

In step (b), the nucleic acid sample is analyzed for the presence and the level (abundance or relative level) of indicator RNA transcripts (mRNA) in the sample. When referring to indicator RNA in a sample, it is clear that this also encompasses indicator cDNA obtainable from said mRNA.

In one embodiment, the mRNA (or cDNA) sequences, which are detected in a sample, and which are indicative of the sensory decay are SEQ ID NO: 57-76, or variants thereof, or fragments of any of these (the main set of indicator genes). Thus, any method may be used to detect the relative or absolute amounts of SEQ ID NO: 57-76, variants of SEQ ID NO: 57-76, or fragments of these in the sample(s). For example, PCR primer pairs which amplify fragments of each of SEQ ID NO: 57-76 may be used in quantitative RT-PCR reactions. Alternatively, the nucleic acid sample may be labeled and hybridized to a nucleic acid carrier comprising oligonucleotides of each of SEQ ID NO: 57-76 (and/or variants thereof), whereby the level of these transcripts in the sample is determined.

In another embodiment a subset of indicator genes is detected in the sample, and the transcript level is compared to the transcript level of the same subset in a suitable control.

SEQ ID NO: 57-66, and variants thereof, are upregulated when sensory decay is initiated, compared to non-decaying fruit (referred to as “upregulated transcripts” indicative of sensory decay). Further, SEQ ID NO: 67-76, and variants thereof, are downregulated when sensory decay is initiated, compared to non-decaying fruit (referred to as “down-regulated transcripts” indicative of sensory decay). Most preferably, the mRNA or cDNA level of a set of at least 2, 3, 4, 5, 6, 7, 8, 9, 10, 15, 18 or more (e.g., 20) of any one of SEQ ID NO: 57-76, and/or variants or fragments thereof, is determined in the sample in step (b). The expression level of the indicator transcripts is preferably compared to the level of transcript of a suitable control, e.g., either the same fruit analyzed at an earlier stage (non decaying), or another suitable control sample, such as the sample of an non-decaying fruit and/or training batches of known decay stages. The method is, thus, especially suitable for discriminating between various fruit batches after harvest, such as non-decaying, slightly decaying, very decaying batches, etc.

In a preferred embodiment the expression level of at least one “upregulated transcript” and one “downregulated transcript” are detected. Thus, the “minimal set” of indicator mRNAs comprises at least two mRNAs, one selected from SEQ ID NO: 57-66 (or variants or fragments thereof) and one selected from SEQ ID NO: 67-76 (or variants or fragments thereof).

As already mentioned, it is understood that also “variants” of SEQ ID NO: 57-76 may be detected in a sample, such as nucleic acid sequences essentially similar to any of SEQ ID NO: 57-76, i.e., comprising at least 70, 75, 80, 85, 90, 95, 98, 99% or more nucleic acid sequence identity to any of SEQ ID NO: 57-76. Such variants may for example be present in different species or different varieties.

The actual method used for determining the level of the set of indicator mRNA transcripts is not important. Any gene expression profiling method may be used, such as RT-PCR, microarrays or chips, Northern blot analysis, cDNA-AFLP, etc. See elsewhere herein. For example, PCR primer pairs for each of SEQ ID NO: 57-76 may be designed using known methods. Suitable primer pairs are, for example, the PCR primer pairs provided in the Examples and depicted in SEQ ID NO: 57-76. In one embodiment of the invention two or more of these primer pairs are used in the method. Alternatively, nucleic acid probes, which hybridize to SEQ ID NO: 57-76 may be made for use in the detection. Any fragment of at least about 10, 12, 15, 20, 22, 30, 50, 100, 200, 300, 500 or more consecutive nucleotides of SEQ ID NO: 57-76, or the complement strand, or of a variant of SEQ ID NO: 57-76, may be suitable for detection of the full length transcript in a sample. Equally, any fragment of a “variant” of any one of SEQ ID NO: 57-76 (as defined above) may be used.

In one embodiment a carrier is provided comprising nucleic acid molecules SEQ ID NO: 57-76, variants of SEQ ID NO: 57-76 and/or most preferably fragments (oligonucleotides) of any of these or of a subset of any of these. The carrier may, for example, be contacted under hybridizing conditions with the (labeled) nucleic acid sample of the sample of step (a), allowing detection of the level of each of the indicator transcripts present in the sample.

If the expression profile of the indicator mRNAs of the fruit corresponds to the profile of fruit which have initiated sensory decay, or which are non-decaying, or at an advanced stage of decay, the plant, fruit or batch can be identified and selected for further use (or for being discarded). Preferably, the fruit or batch of fruit can be selected and removed from non-decaying fruit or batches.

In a further embodiment, kits, oligonucleotides (e.g., PCR primers, nucleic acid probes) and antibodies are provided, for determining the stage of sensory decay of fruit. Such kits comprise instructions for use and one or more reagents for use in the method. Optionally, tissue samples or nucleic acid samples suitable as controls may be included. Thus, such a kit may comprise a carrier to receive therein one or more containers, such as tubes or vials. The kit may further comprise unlabeled or □abelled oligonucleotide sequences of the invention (SEQ ID NO: 57-76, or variants thereof, or parts thereof, such as primers or probes), e.g., to be used as primers, probes, which may be contained in one or more of the containers, or present on a carrier. The oligonucleotides may be present in lyophilized form, or in an appropriate buffer. One or more enzymes or reagents for use in isolation of nucleic acids, purification, restriction, ligation and/or amplification reactions may be contained in one or more of the containers. The enzymes or reagents may be present alone or in admixture, and in lyophilised form or in appropriate buffers. The kit may also contain any other component necessary for carrying out the present invention, such as manuals, buffers, enzymes (such as preferably reverse transcriptase and a thermostable polymerase), pipettes, plates, nucleic acids (preferably labelled probes), nucleoside triphosphates, filter paper, gel materials, transfer materials, electrophoresis materials and visualization materials (preferably dyes, labelled antibodies or -enzymes) autoradiography supplies.

### Assays and Kits for the Prediction of Brown Discoloration in Edible Mushrooms

Edible mushrooms, such as Agaricus bisporus, are consumed worldwide, both as fresh product or processed in pots, canned, frozen etc. For many mushrooms, and especially white mushrooms, product quality is generally judged visually, based on colour. Fresh Agaricus bisporus has a white cap and stalk, but the colour of the cap or the gills can unexpectedly change to a light or darker brown colour, lowering the product's quality. Thus, a test to predict the quality, days before severe browning will occur, would be of great potential value for the mushroom industry.

So far, no such tests are available. A visually screen by growers or inspectors does not give a conclusive prediction. Also computer image analysis has been tried but also these do not give solid predictions about how fast the mushroom product will decay.

The present inventors were able to develop an assay to indicate the freshness stage of mushrooms and the product quality stage prior to visual sign, such as browning of the cap. Twenty-three indicator genes depicted in SEQ ID NO: 113-135 (of which 7 sequences, depicted in SEQ ID NO: 113-119, where previously published by other investigators), were selected, whose expression correlates with browning of the tissue. Thus, a specific expression profile of these indicator genes in a sample indicates, relative to other batches, what the time span is in which the batch is predicted to start browning.

Thus, in one aspect of the invention a method is provided for detecting early changes in relative expression levels of indicator genes, which serve as an early warning for browning in edible mushrooms. Using methods which rely on visual symptoms of browning one can only detect deterioration of mushroom quality once it is already visible. In the present method much earlier signs of quality loss can be determined.

In one embodiment a method for detecting the quality stage (browning stage) of edible mushrooms, especially of edible homobasidiomycetes, such as edible species of the families Agaricaceae, Tricholomataceae, Lepista, Pleurotaceae, Cantharellaceae, and Boletaceae, is provided. Most preferably, the method is used in Agaricus species, especially Agaricus bisporus, and in shiitake (Lentinus edodes), Pleurotus ostreatus (Oyster mushroom), Lepista nuda (synonyms Clitocybe nuda, Tricholoma nudum en Rhodopaxillus nudus) which are close relatives of Agaricus bisporus, as well as Cantharellus cibarius and Boletus edulis.

The method provided herein a set of 23 indicator genes whose expression profile can be used as measurement for the (relative) browning stage of fresh mushrooms and fresh mushroom based products. Based on the relative or absolute expression level of the described indicator genes conclusions can be drawn about the stage of browning and freshness of the mushroom that is reached post-harvest.

As shown in the Examples, comparison of expression levels of a set of 23 genes correlates with browning stages, prior to visible browning being seen. Thus, discrimination between batches which are starting to develop browning (although not yet visible) and between good quality batches is possible.

The method for detecting signs of quality loss (initiation of browning) of edible mushrooms, especially homobasidiomycetes, comprises the following steps:
(a) providing a nucleic acid sample (comprising mRNA or cDNA) of a mushroom or mushroom tissue (or a plurality of mushrooms or mushroom tissues; batch or batches),
(b) analyzing the sample by determining the level of a set of indicator mRNA transcripts in the sample, which are indicative of the quality stage of the mushrooms, and optionally
(c) identifying and selecting the mushrooms which comprises a certain level of the indicator mRNA transcripts, relative to suitable controls, for further use, e.g., for immediate processing or sale.

Thus, mushrooms which comprise an “indicator mRNA profile” which is indicative that the mushrooms (or batch) has already initiated browning allows mushrooms or batches comprising a more advanced browning stage to be differentiated and removed from mushrooms which show no signs of quality loss. Also the browning stage during storage can be followed using the method, allowing the discrimination between batches, which are at different browning stages. Similarly, mushroom production and storage conditions can be optimized, by testing the effect of various parameters (temperature, humidity, compost etc.) on the browning process of mushrooms.

The method can be applied to determine the browning stage of mushrooms of the above families. Most preferably, it is applied to white mushrooms, especially Agaricus bisporus.

Any tissue of the mushroom may be used in the method, for example the cap, stem, or any other part of the fruiting body. Preferably cap tissue is used to prepare the nucleic acid sample. To have a good coverage of the potency of the whole batch, preferably at least about 10-20 fruiting bodies are sampled randomly from the batch. Definition of a batch is a product, sampled on the same day from the same climate room and that have been treated the same from harvest until sampling. Thus, first suitable tissue is sampled for nucleic acid extraction. In the present method, it is preferred that in step (a) nucleic acid samples are prepared by harvesting mushroom samples, grind and mix sample material and extracting the total RNA or total mRNA from the sample. The sample can be prepared using known nucleic acid extraction methods, e.g., total RNA or mRNA purification methods and kits provided in the art (e.g., RNAeasy kits of Qiagen, kits of SIGMA, Clonetech, etc.). The mRNA may be reverse transcribed into cDNA, using known methods. Preferably expression levels of the indicator genes are analyzed relative to the expression level of the indicator genes in a training set of batches having a known browning stage (see herein below). Having a training set of at least about 30, preferably at least about 45 samples, e.g., at least about 10 or at least about 15 from three ‘browning stage’ classes, the genes expression of new ‘test’ batches is determined relative to the gene expression of the indicator genes in the training set batches, to predict in which quality class the new ‘test’ batches fit best.

In step (b), the nucleic acid sample is analyzed for the presence and the level (abundance or relative level) of indicator RNA transcripts (mRNA) in the sample. When referring to indicator RNA in a sample, it is clear that this also encompasses indicator cDNA obtainable from said mRNA.

In one embodiment, the mRNA (or cDNA) sequences, which are detected in a sample, and which are indicative of the browning (or predicted browning) are SEQ ID NO: 113-135, or variants thereof, or fragments of any of these (the main set of indicator genes). Thus, any method may be used to detect the relative or absolute amounts of SEQ ID NO: 113-135, variants of SEQ ID NO: 113-135, or fragments of these in the sample(s). For example, PCR primer pairs which amplify fragments of each of SEQ ID NO: 113-135 may be used in quantitative RT-PCR reactions. Alternatively, the nucleic acid sample may be labeled and hybridized to a nucleic acid carrier comprising oligonucleotides of each of SEQ ID NO: 113-135, whereby the level of these transcripts in the sample is determined.

In another embodiment a subset of indicator genes is detected in the sample, and the transcript level is compared to the transcript level of the same subset in a suitable control. Most preferably, the mRNA or cDNA level of a set of at least 2, 3, 4, 5, 8, 10, 15, 18 or more (e.g., 20) of any one of SEQ ID NO: 113-135, and/or variants or fragments thereof, is determined in the sample in step (b). The expression level of the indicator transcripts is preferably compared to the level of transcript of a suitable control, e.g., either the same mushroom analyzed at an earlier stage, or another suitable control sample, such as the sample of a fresh, white mushroom.

As already mentioned, it is understood that also “variants” of SEQ ID NO: 113-135 may be detected in a sample, such as nucleic acid sequences essentially similar to any of SEQ ID NO: 113-135, i.e., comprising at least 70, 75, 80, 85, 90, 95, 98, 99% or more nucleic acid sequence identity to any of SEQ ID NO: 113-135. Preferably, the putative linker sequence present at the 5′ end (as shown in the Sequence Listing) is removed prior to sequence alignment. Such variants may for example be present in different species or different varieties of edible mushrooms.

The actual method used for determining the level of the set of indicator mRNA transcripts is not important. Any gene expression profiling method may be used, such as RT-PCR, microarrays or chips, Northern blot analysis, cDNA-AFLP, etc. See elsewhere herein. For example, PCR primer pairs for each of SEQ ID NO: 113-135 may be designed using known methods. Alternatively, nucleic acid probes, which hybridize to SEQ ID NO: 113-135 may be made for use in the detection. Any fragment of at least about 10, 12, 14, 15, 20, 22, 30, 50, 100, 200, 300, 500 or more consecutive nucleotides of SEQ ID NO: 113-135, or the complement strand, or of a variant of SEQ ID NO: 113-135, may be suitable for detection of the full length transcript in a sample. Equally, any fragment of a “variant” of any one of SEQ ID NO: 113-135 (as defined above) may be used.

In one embodiment a carrier is provided comprising nucleic acid molecules SEQ ID NO: 113-135, variants of SEQ ID NO: 113-135 and/or most preferably fragments (oligonucleotides) of any of these or of a subset of any of these. The carrier may, for example, be contacted under hybridizing conditions with the (labeled) nucleic acid sample of the sample of step (a), allowing detection of the level of each of the indicator transcripts present in the sample.

If the expression profile of the indicator mRNAs of the mushrooms corresponds to the profile of mushrooms in a training set which is known to have good postharvest storability potency (days until visual browning appears), the new ‘test’ mushrooms are likely also to have good storability capacity (e.g., at least about 5, 6, 7 or more days at 2° C.) without visible signs of browning developing.

When the expression levels of the indicator sequences is analyzed and the expression of the indicator genes is such that it fits the expression levels of the batches of a training set labeled as ‘moderate storability’ (measured using exactly the same method, using the same protocol and software programs, such as e.g., Predicted Analysis of Microarray or PAM), it is very likely that the new ‘test’ mushroom material also will have relative moderate post-harvest storability potency (e.g., it can be stored at least about 2-5 days at 2° C. without developing visible browning).

When the expression levels of the indicator sequences is analyzed and the expression of the indicator genes is such that it fits the expression levels of the batches of a training set labeled as ‘bad’ (measured using exactly the same method, using the same protocol and software programs, such as e.g., Predicted Analysis of Microarray), it is very likely that the new ‘test’ mushroom material also will have relative low storability potency and can only be stored only for a very short time without browning (e.g., it can be stored for two days or for less than 2 days at 2° C. without developing visible browning).

These batch quality indications can be used to select batches for specific markets, like far away countries with long logistic track (good quality), markets that require high quality mushrooms, local markets or discount markets, value packages (moderate quality) or processing industry (low quality).

In a further embodiment, kits, oligonucleotides (e.g., PCR primers, nucleic acid probes) and antibodies are provided, for determining the stage of browning. Such kits comprise instructions for use and one or more reagents for use in the method. Optionally, tissue samples or nucleic acid samples suitable as controls may be included. Thus, such a kit may comprise a carrier to receive therein one or more containers, such as tubes or vials. The kit may further comprise unlabeled or labelled oligonucleotide sequences of the invention (SEQ ID NO: 113-135, or variants thereof, or parts thereof, such as degenerate primers or probes), e.g., to be used as primers, probes, which may be contained in one or more of the containers, or present on a carrier. The oligonucleotides may be present in lyophilized form, or in an appropriate buffer. One or more enzymes or reagents for use in isolation of nucleic acids, purification, restriction, ligation and/or amplification reactions may be contained in one or more of the containers. The enzymes or reagents may be present alone or in admixture, and in lyophilised form or in appropriate buffers. The kit may also contain any other component necessary for carrying out the present invention, such as manuals, buffers, enzymes (such as preferably reverse transcriptase and a thermostable polymerase), pipettes, plates, nucleic acids (preferably labelled probes), nucleoside triphosphates, filter paper, gel materials, transfer materials, electrophoresis materials and visualization materials (preferably dyes, labelled antibodies or -enzymes) autoradiography supplies.

Assays and Kits for the Determination/Prediction of Post-harvest Loss of Firmness in Solanaceous Fruit, such as Tomatoes

Post-harvest quality loss in fleshy fruits, such as tomatoes, can be separated into various components. One of the most prominent component is loss of firmness. The biological variation with respect to this characteristic between cultivars and between batches of the same cultivar is large. Genetic variation has been explored to a large extent by breeders and has resulted in tomato cultivars that produce fruit with long tenability. However, even in the best-performing tomato cultivars the intra-cultivar variation can still result in batches of fast-softening tomatoes. FIG. 5.1 shows the biological variation with respect to firmness in tomatoes with the same genetic background (cultivar Aromata) but cultured by different growers and in different seasons. In addition, extended firmness is often associated with a decrease in flavor and aroma components, resulting in the recent trend towards softer, less tenable fruits, such as tomatoes. Both the use of cultivars with shorter shelf-lives and the non-genetic, environmentally induced biological variation enhance the need for reliable quality monitoring tools for use in trade and distribution of fresh fruits, such as tomatoes.

Tests available at present can be used for monitoring actual firmness, but do not allow predicting future firmness. The future firmness is the most important factor in deciding on distribution chains for harvested fleshy fruit batches, e.g., tomato batches.

Herein a method is provided which uses a set of 19 indicator genes and optionally 3 control genes to predict the post-harvest firmness development of fruits from Solanaceous species, especially tomatoes. Based on the expression level of the indicator genes conclusions can be drawn about the predicted quality class of a batch of fruit, e.g., a batch of tomatoes. For tomatoes, firmness development is generally assessed every 2 or 3 days during a 4 week period. During this period the batches are stored in climate-controlled rooms at 18° C. and 75% relative humidity. These conditions were chosen in the Examples to induce a decrease in quality at a moderate speed that would allow for accurate measurements of loss of firmness and gene expression levels over time.

“Firmness” of harvested fruit can be assessed using physical means and can be determined for example on a scale of 2 to 8 (Sensoric values, with 2 being very soft, 5 being firm and 8 being extra hard; see Examples) and/or Instron values on a scale of 0 to −1.3 mm (whereby 0 mm is extra hard, −0.6 mm is firm and −1.3 mm is very soft; see Examples). The critical sensoric firmness value of about 5 (corresponding to the Instron value of about −0.6 mm) has been found to be the lowest firmness level that is still acceptable to consumers. The sensoric value of 5 is therefore referred to as the “critical firmness value” herein, at least for tomatoes. The critical firmness value may be different for other fruits, but can be established by the skilled person.

The quality class labeled ‘good’ refers to batches of tomatoes that have a shelf life of about 28 days or more before they drop below the critical firmness value of 5. Quality class labeled ‘average’ is used for batches having between about 15 and 28 days shelf life. Batches labeled as ‘bad’ refer to batches that drop below the critical firmness value of 5 within about 15 days after harvest.

In one aspect of the invention a method is provided for determining and predicting the post-harvest firmness development (or ‘loss of firmness’; development of ‘softness’) of Solanaceous fruits, preferably tomatoes (Solanum lycopersicum), but also of other Solanaceous fruit, such as peppers (Capsicum annuum; Capsicum frutescens, etc.) and aubergines (Solanum melongena). The mRNA levels of a set of indicator genes, thus, serve as an indicator of the quality of the fruit with respect to firmness loss and one can determine early on whether a batch of fruit has a long or short shelf life and a slow or rapid loss of firmness respectively.

Thus, in one embodiment a method for determining the firmness development of fruits of the family Solanaceae, especially of the genera Capsicum and Solanum, is provided.

The method provided herein uses a set of 19 indicator genes whose expression profile can be used as measurement of the likelihood that the fruits will loose firmness faster than average. Based on the relative or absolute expression level of the described indicator genes conclusions can be drawn about the quality of plants or plant parts regarding their predicted firmness decrease during post-harvest storage.

As shown in the Examples, comparison of expression levels of a set of 19 genes in various batches of tomatoes provided an indication of the future firmness decrease of a fruit or batch, under conditions similar to storage conditions in practice. Thus, early discrimination between batches which are of “poor” quality (likely to show rapid decrease in firmness) and “good” quality (likely to show slow decrease in firmness) is possible.

The method for determining (predicting) the future firmness loss of fruits (especially tomato fruits) of the family Solanaceae comprises the following steps:
(a) providing a nucleic acid sample (comprising mRNA or cDNA) of a plant tissue (or a plurality of plant tissues; batch),
(b) analyzing the sample by determining the level of a set of indicator mRNA transcripts in the sample, which are indicative of the firmness development of the fruit or batch, and optionally
(c) identifying and selecting the plant or plant parts or batch which comprises a certain level of the indicator mRNA transcripts, relative to suitable controls, for further use, e.g., good quality batches can be transported or sold or stored for longer (as they soften slowly and have a longer shelf life), while bad quality batches can be destroyed or sold immediately (as they soften faster and have a shorter shelf life).

Thus, plants or plant parts which comprise an “indicator mRNA profile” which is indicative of the post-harvest firmness development can be differentiated and handled differently.

Preferably, the method is carried out once (or several times, e.g., at regular time intervals, such as once every two days, once a week, etc.) after harvest, in order to sort plants or batches into different groups based on prediction of firmness development.

Any tissue of the fruit may be used in the method, for example pericarp, mesocarp, stem etc., although preferably, the mesocarp is used to prepare the nucleic acid sample. To have a good coverage of the potency of the whole batch, preferably at least about 15, more preferably at least about 20 individual fruits are sampled randomly from the batch. Definition of a batch is a product, sampled at the same day from the same greenhouse that have been treated the same from harvest until sampling. Thus, first suitable tissue is sampled for nucleic acid extraction. In the present method, it is preferred that in step (a) nucleic acid samples are prepared by harvesting mesocarp samples of a fruit, grind and mix sample material and extracting the total RNA or total mRNA from the sample. The sample can be prepared using known nucleic acid extraction methods, e.g., total RNA or mRNA purification methods and kits provided in the art (e.g., RNAeasy kits of Qiagen, kits of SIGMA, Clonetech, etc.). The mRNA may be reverse transcribed into cDNA, using known methods. Expression levels of the indicator genes are preferably analyzed relative to a training set of batches (preferably same plant materials and same cultivar) with known development of firmness over time in a shelf-life test. Having a training set of at least about 25 or 30, preferably at least about 40 or 45 samples, i.e., at least about 10, 12, or more, e.g. 15 samples from each quality class (for example 15 samples from a ‘good’ class and 15 samples from a ‘bad’ class, as described above), the gene expression of new ‘test’ batch(es) is then analyzed relative to the indicator gene expression of the training set batches to predict in which quality class the new ‘test’ batch(es) fit(s) best.

In step (b), the nucleic acid sample is analyzed for the presence and the level (abundance or relative level) of indicator RNA transcripts (mRNA) in the sample. When referring to indicator RNA in a sample, it is clear that this also encompasses indicator cDNA obtainable from said mRNA. Preferably Real time RT-PCR using primers which amplify the indicator transcripts (or a subset thereof) is used as described in the Examples.

In one embodiment, the mRNA (or cDNA) sequences, which are detected in a sample, and which are indicative of the firmness development of the tissue are selected from or consist of SEQ ID NO: 136-154, optionally including one or more of SEQ ID NO: 155-157, or variants or fragments of any of these. SEQ ID NO: 136-154 (or variants or fragments thereof) is herein referred to as the “main set” of indicator genes. Thus, any method may be used to detect the relative or absolute amounts of SEQ ID NO: 136-154 or variants or fragments of these in the sample(s). For example PCT primer pairs which amplify fragments of each of SEQ ID NO: 136-154 may be used in qPCR reactions. Alternatively, the nucleic acid sample may be labeled and hybridized to a nucleic acid carrier comprising oligonucleotides of each of SEQ ID NO: 136-154 (and optionally 155-157; and/or variants of any of these) whereby the level of these transcripts in the sample is determined. Expression levels may be normalized against the expression levels of genes having a “constant” expression during fruit storage, such as e.g., those of SEQ ID NO: 155-157.

In another embodiment a subset of indicator genes is detected in the sample, and the transcript level is compared to the transcript level of the same subset of indicator genes in a suitable control. A subset may comprise any subset of SEQ ID NO: 136-154 (or variants thereof), such as the detection of 20, 15, 10, 6, 5, 4, 3 or less (e.g., 3) of the sequences.

SEQ ID NO: 136-144, and variants thereof, are down-regulated in poor quality batches (referred to as “down-regulated transcripts indicative of rapid loss of firmness and shorter shelf-life), i.e., batches which are predicted to develop a rapid loss of firmness during storage. Further, SEQ ID NO: 145-154 are up-regulated in poor quality batches (referred to as “up-regulated transcripts indicative of rapid loss of firmness and shorter shelf-life).

In a preferred embodiment the expression level of at least one, preferably at least 1, 2, 3, 4 or 5 of the “down-regulated” transcripts and at least one, preferably at least 1, 2, 3, 4 or 5 of the “up-regulated” transcripts is determined. Therefore, a “minimal set” of indicator mRNA transcripts preferably comprises at least 2 transcripts, one from the up-regulated set and one from the down-regulated set.

The expression profile of SEQ ID NO: 136-154, and/or variants thereof, predicts the speed with which firmness decreases in the 4 to 6 weeks after harvest. Thus, when the expression levels of the indicator sequences is analyzed and the expression of the indicator genes is such that it fits the expression levels of the batches of the training set labeled as ‘good’ (measured using exactly the same method, using the same protocol and using software programs like Predicted Analysis of Microarray), it is very likely that the new tested plant material also will have slow decrease of firmness (drops below the value of 5 after 28 days or more) as was found for the batches in quality class ‘good’ of the train set.

When the expression levels of the indicator sequences is analyzed and the expression of the indicator genes is such that it fits the expression levels of the batches of the train set labeled as ‘moderate’ (measured using exactly the same method, using the same protocol and using software programs like Predicted Analysis of Microarray), it is very likely that the new tested plant material also will have relative moderate development of firmness loss (drops below critical value of 5 between 15 and 28 days) during post-harvest shelf-life as was found for the batches in quality class ‘moderate’ of the train set.

When the expression levels of the indicator sequences is analyzed and the expression of the indicator genes is such that it fits the expression levels of the batches of the train set labeled as ‘bad’ (measured using exactly the same method, using the same protocol and using software programs like Predicted Analysis of Microarray), it is very likely that the new tested plant material also will have relative fast development of firmness loss (drops below critical value of 5 within 15 days) during post-harvest shelf-life as was found for the batches in quality class ‘bad’ of the train set.

In a preferred embodiment the “minimal set” of indicator mRNAs comprises at least 2, 3, 4, 5, 6, 7, 8, 9, 10, 15, 20 or more mRNAs selected from SEQ ID NO: 136-154 (or variants or fragments thereof). Preferably, a “minimal set” comprises at least one “upregulated” and at least one “down-regulated” transcript, as described above.

As already mentioned, it is understood that also “variants” of SEQ ID NO: 136-154 may be detected in a sample, such as nucleic acid sequences essentially similar to any of SEQ ID NO: 136-154, i.e., comprising at least 70, 75, 80, 85, 90, 95, 98, 99% or more nucleic acid sequence identity to any of SEQ ID NO: 136-154. Such variants may for example be present in different species or different varieties.

The actual method used for determining the level of the set of indicator mRNA transcripts is not important. Any gene expression profiling method may be used, such as RT-PCR, microarrays or chips, Northern blot analysis, cDNA-AFLP, etc. See elsewhere herein. For example, PCR primer pairs for each of SEQ ID NO: 136-154 or variants thereof, optionally also to one or more of SEQ ID NO: 155-157 or variants thereof, may be designed using known methods. In one embodiment of the invention two or more of these primer pairs are used in the method. Alternatively, nucleic acid probes, which hybridize to SEQ ID NO: 136-154, or variants thereof, may be made for use in the detection. Any fragment of at least about 10, 12, 14, 15, 20, 22, 30, 50, 100, 200, 300, 500 or more consecutive nucleotides of SEQ ID NO: 136-154, or the complement strand, or of a variant of SEQ ID NO: 136-154, may be suitable for detection of the full length transcript in a sample. Equally, any fragment of a “variant” of any one of SEQ ID NO: 136-154 (as defined above) may be used.

In one embodiment a carrier is provided comprising nucleic acid molecules selected from SEQ ID NO: 136-154, variants of SEQ ID NO: 136-154 and/or most preferably fragments (oligonucleotides) of any of these or of a subset of any of these. The carrier may, for example, be contacted under hybridizing conditions with the (labeled) nucleic acid sample of the sample of step (a), allowing detection of the level of each of the indicator transcripts present in the sample.

If the expression profile of the indicator mRNAs of the fruit corresponds to the profile of fruit which are prone to rapid firmness loss, the fruit or batch can be identified and selected for further use. Preferably, the fruit or batch of fruit can be selected and removed from non-decaying fruit or batches or treated differently or be destined for shorter post-harvest distribution chains.

In a further embodiment, kits, oligonucleotides (e.g., PCR primers, nucleic acid probes) and antibodies are provided, for determining the firmness loss of harvested fruits. Such kits comprise instructions for use and one or more reagents for use in the method. Optionally, tissue samples or nucleic acid samples suitable as controls may be included. Thus, such a kit may comprise a carrier to receive therein one or more containers, such as tubes or vials. The kit may further comprise unlabeled or labelled oligonucleotide sequences of the invention (SEQ ID NO: 136-154, or variants thereof, or parts thereof, such as degenerate primers or probes), e.g., to be used as primers, probes, which may be contained in one or more of the containers, or present on a carrier. The oligonucleotides may be present in lyophilized form, or in an appropriate buffer. One or more enzymes or reagents for use in isolation of nucleic acids, purification, restriction, ligation and/or amplification reactions may be contained in one or more of the containers. The enzymes or reagents may be present alone or in a mixture, and in lyophilised form or in appropriate buffers. The kit may also contain any other component necessary for carrying out the present invention, such as manuals, buffers, enzymes (such as preferably reverse transcriptase and a thermostable polymerase), pipettes, plates, nucleic acids (preferably labelled probes), nucleoside triphosphates, filter paper, gel materials, transfer materials, electrophoresis materials and visualization materials (preferably dyes, labelled antibodies or -enzymes) autoradiography supplies.

### DESCRIPTIONS OF THE DRAWINGS

FIG. 1.1: Frost tolerance determined by electrolyte leakage (see Example 1)

FIG. 1.2 A and B: indicator gene expression (see Example 1)

FIG. 2.1: Firmness of pears from two orchards harvested at two days intervals in September 2003.

FIG. 2.2: Relative expression levels (normalized against the actin gene) of selected genes measured during a part of the period described in FIG. 2.1 and in one orchard.

FIG. 2.3: Expression level of three indicator genes in pears, namely ACC oxidase (SEQ ID NO: 43), Galactosidase (SEQ ID NO: 163) and SAM synthase (SEQ ID NO: 42), as well as firmness levels (kg/cm²) and starch levels (using a color chart with a scale of 1-10).

FIG. 3.1: PCA plot of sensory characteristics of batches of apples stored at two different temperatures for various periods. Data from two trials are displayed (FI and FII).

FIG. 3.2: Firmness data from trial FII. Significant differences between apples stored at 4 and 18° C. can only be measured after two weeks of storage

FIG. 3.3: PCA plot of the various batches of apples based on results from expression analysis of 900 genes.

FIG. 4.1: Clearness, whiteness, % browning and diameter of the white button mushrooms selected for microarray analysis.

FIG. 4.2: Hierarchical clustering of the selected genes and physiological post-harvest data (whiteness, lightness and browning day 7).

FIG. 4.3: Pedigree of Homobasidiomycete, to which Agaricus bisporus belongs.

FIG. 5.1: Biological variation in shelf life in batches tomato fruit from cultivar Aromata harvested in April, June, August and September 2003, April and September 2004 and May 2005. At each harvest date tomatoes were obtained from various growers. Shelf life period is defined as the number of days needed before the average firmness value for the batch drops below the arbitrary critical value of 5. This is the value below which batches can no longer be sold to retailers.

FIG. 5.2: Typical post-harvest development of firmness (diamonds) and colour squares) over time. Firmness and colour are indicated with arbitrary units that refer to standards used in Dutch practice.

FIG. 5.3: Predictive analysis of tomato samples. For each sample the firmness development was determined and they were classified as having low (triangle), average (square) or good (diamond) accordingly. Subsequently gene expression profiles of the same samples taken at the start of the shelf life period, when all samples had equivalent firmness, were used to perform a prediction of quality. The figure shows that most samples are placed in the right class. When a yellow triangle is placed at 0.9 height in the right section of the graph, this should be interpreted as a likelihood of 95% that the sample has low quality.

FIG. 6.1: PAM analysis of 18 different batches of Rose, classified good (percentage of flowers with visible Botrytis infection after 7 days vase life lower than 20%) and bad. Prediction based on expression data of 22 genes (indicated in Table 8) shows that in most cases cross validation gives a reliable result (probability higher than 0.8).

FIG. 6.2: Expression ratio of ORoseR0626/ORoseR0277 (SEQ ID NO:106 to SEQ ID NO:102 mRNA ratio) in batches of Rose from four different cultivars that develop various degrees of visible Botrytis infection after 7 days vase life.

### SEQUENCES

SEQ ID NO 1-29: upregulated, downregulated and constant beech seedling sequences;
SEQ ID NO 30-41: PCR primer pairs for amplification of:
   transcript SEQ ID NO:9 (primers of SEQ ID NO:30 and 31);
   transcript SEQ ID NO:11 (primers of SEQ ID NO:32 and 33);
   transcript SEQ ID NO:1 (primers of SEQ ID NO:34 and 35);
   transcript SEQ ID NO:20 (primers of SEQ ID NO:36 and 37);
   transcript SEQ ID NO:24 (primers of SEQ ID NO:38 and 39);
   transcript SEQ ID NO:28 (primers of SEQ ID NO:40 and 41);
SEQ ID NO 42-46: indicator-gene transcripts of pears;
SEQ ID NO 47-56: PCR primer pairs for amplification of pear indicator transcripts;
SEQ ID NO 57-76: upregulated and downregulated apple mRNA sequences;
SEQ ID NO 77-109: Rose indicator mRNA sequences;
SEQ ID NO 110-112: Rose housekeeping mRNA sequences;
SEQ ID NO 113-135: Agaricus indicator mRNA sequences. SEQ ID NO:120-135 contain putative SSH linker sequences at the 5′ end (indicated), which are preferably removed prior to sequence alignments or for detection purposes.
SEQ ID NO 136-154: Solanaceae indicator mRNA sequences from tomato. SEQ ID NO:136-144 are downregulated in low quality batches, while SEQ ID NO:145-154 are upregulated in low quality batches.
SEQ ID NO 155-157: additional Solanaceae indicator mRNA sequences, comprising a constant mRNA expression level during post-harvest storage.
SEQ ID NO:158-171: additional pear indicator mRNA sequences.
SEQ ID NO:172-174 additional rose indicator mRNA sequences

### EXAMPLES

### Example 1

### Quality Assay for Determining Cold Tolerance in Fagaceae, Exemplified by Fagus sylvatica L. Seedlings (Beech)

1.1 Indicator genes

A set of 29 indicator genes (SEQ ID NO:1-29) have been selected whose expression profile can be used as measurement for cold tolerance level of beech seedlings.

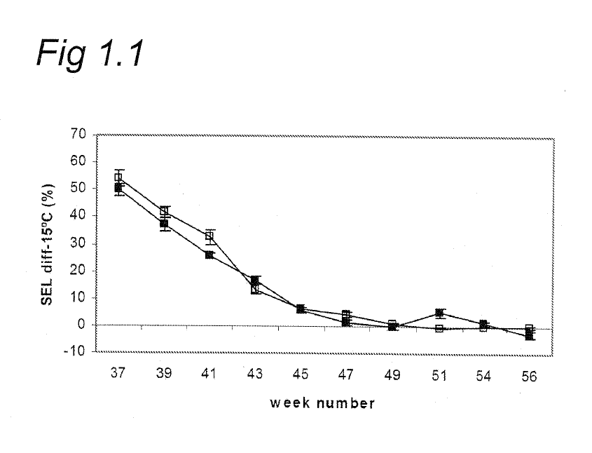

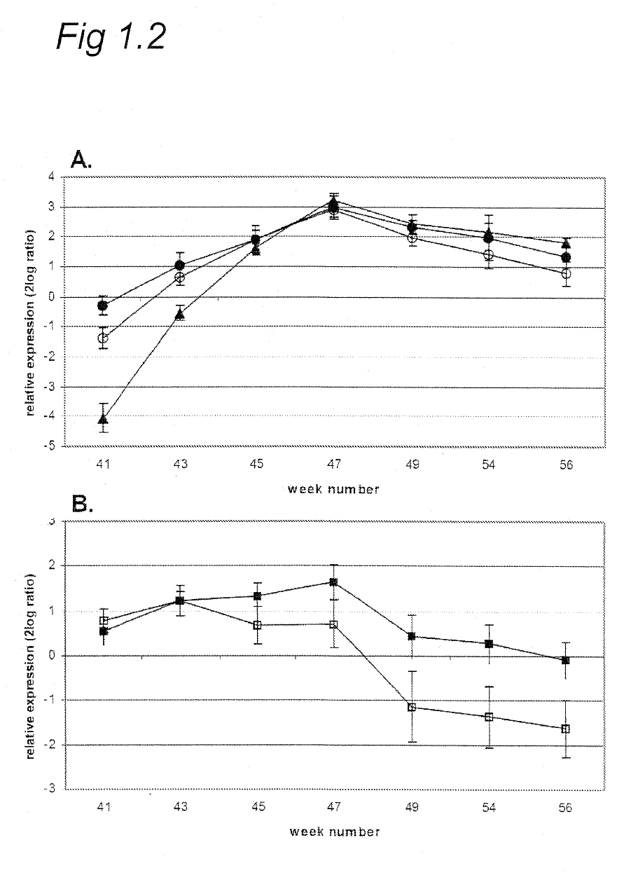

Based on the expression level of the described genes conclusions can be drawn about the level of frost tolerance that is reached in beech seedlings. As soon as the expression of the frost tolerance related genes stabilises at high levels, frost tolerance has reached the maximal level (FIGS. 1.1 and 1.2).

FIG. 1.1 shows typical frost tolerance pattern of two batches of one-year-old beech seedlings, planted at two different locations (Scotland, black squares and Denmark, open squares), season 2001/2002. Tolerance is defined as percentage of electrolyte leakage (SEL) as a result of freezing until −15° C. When SEL diff-values fall below 10%, seedlings are considered to be completely frost tolerant. In this case seedlings are frost tolerant from week 45 on.

FIG. 1.2 shows the expression patterns of groups of indicator genes selected after hybrizations using a microarray. Groups consist of genes that showed the same expression patterns in both batches described in FIG. 1.1. Selected indicator sequences are derived from both groups; upregulated genes (A.) and downregulated genes (B.).

Using indicator genes and a proper test setup, results can be generated within one day. Table 1 and Table 2 show the indicator gene expression data. Data with the code 704 is 1 year old beech seedling plant material from a field in Scotland (2001/2002). Data with the code 406 is 1 year old plant material from a field in Denmark (2001/2002). WK indicates the week.

TABLE 1

| | | | | | | | |
|---|---|---|---|---|---|---|---|
| Gene | 704wk37 | 740wk41 | 704wk43 | 704wk45 | 704wk47 | 704wk51 | 704wk04 |

Frost tolerance upregulated genes (SEQ ID NO: 1-15)

| | | | | | | | |
|---|---|---|---|---|---|---|---|
| b1nr013 | −0.742 | −1.0665 | 0.62 | 1.6715 | 1.287 | 1.1155 | −0.0135 |
| b1nr031 | −0.3855 | −1.4625 | −0.6785 | 0.5275 | 0.9095 | 0.601 | −0.421 |
| b1nr039 | −1.667 | −1.004 | 1.0465 | 1.7605 | 1.8405 | 0.5895 | 0.332 |
| b4nr049 | −1.595 | −0.909 | 0.9385 | 1.7255 | 1.597 | 0.5985 | 0.261 |
| b4nr081 | 0.685 | 0.119 | 1.196 | 2.598 | 1.953 | 1.817 | 1.065 |
| b4nr096 | 0.614 | 0.0415 | 1.274 | 2.6205 | 1.8345 | 1.9235 | 1.3495 |
| b5nr012 | 0.124 | −0.4045 | 0.964 | 1.8355 | 1.2145 | 1.0425 | 1.0295 |
| b5nr018 | −1.6505 | −1.276 | −0.119 | 1.3115 | 1.562 | 1.487 | 0.2135 |
| b5nr019 | −3.446 | −3.0855 | −0.184 | 1.608 | 1.98 | 2.0435 | 0.817 |
| b5nr052 | −0.5365 | −0.57 | 0.5625 | 1.423 | 1.233 | 1.363 | 0.6995 |
| b5nr078 | 0.15375 | 0.98125 | 1.862 | 1.9135 | 1.662 | 1.65 | 0.71425 |
| b6nr008 | −1.675 | −1.1715 | 0.7695 | 1.548 | 1.654 | 0.4545 | 0.123 |
| b6nr046 | −0.5985 | −0.022 | 0.7945 | 1.9615 | 1.8125 | 2.0745 | 0.7005 |
| b6nr057 | −0.411 | −0.046 | 0.308 | 2.114 | 1.749 | 2.441 | 1.6495 |
| b6nr061 | −0.1815 | 0.1395 | 0.486 | 1.8925 | 1.803 | 2.1965 | 1.255 |

Frost tolerance downregulated genes (SEQ ID NO: 16-27)

| | | | | | | | |
|---|---|---|---|---|---|---|---|
| b1nr005 | 0.9655 | 2.5035 | 1.0975 | 0.184 | −1.7615 | −1.613 | −1.671 |
| b1nr019 | 0.5855 | 1.247 | 0.476 | 0.08 | −0.2005 | −0.384 | −1.0045 |
| b1nr025 | 1.072 | 1.837 | 0.63 | 0.2925 | −1.241 | −1.138 | 0.4295 |
| b1nr082 | 0.7775 | 0.7205 | 0.132 | −0.308 | −0.2205 | −0.9535 | −0.742 |
| b2nr070 | 0.922 | 1.3905 | 0.9605 | 0.8755 | 0.2645 | 0.173 | −0.2945 |
| b2nr074 | 1.114 | 1.1255 | 0.9965 | 0.7465 | 0.0885 | −0.3255 | −0.293 |
| b3nr031 | 0.621 | 0.6195 | 0.563 | −0.7715 | −1.695 | −2.3195 | −1.1815 |
| b3nr056 | 0.7615 | 0.567 | 0.424 | 0.218 | −0.1285 | −0.8065 | −0.6275 |
| b3nr058 | 0.8255 | 1.15275 | 0.87925 | 0.365 | −0.5085 | −0.79375 | −0.6185 |
| b3nr083 | 1.319 | 1.0715 | 0.8255 | 0.948 | 0.427 | 0.4205 | 0.425 |
| b3nr095 | 0.99175 | 0.71275 | 0.67825 | 0.645 | 0.28625 | 0.097 | 0.2415 |
| b6nr003 | 0.8605 | 0.683 | 0.686 | 1.13 | 0.4305 | 0.4605 | −0.2845 |

Genes with stable expression (SEQ ID NO: 28 and 29)

| | | | | | | | |
|---|---|---|---|---|---|---|---|
| b3nr038 | 0.698 | 0.078 | −0.268 | −0.3045 | −0.202 | −0.296 | −0.644 |
| b4nr068 | 1.7015 | 1.719 | 1.5975 | 1.861 | 1.268 | 0.9425 | 1.2175 |

TABLE 2

| | | | | | | | |
|---|---|---|---|---|---|---|---|
| Clone | 406wk41 | 406wk43 | 406wk45 | 406wk47 | 406wk49 | 406wk02 | 406wk04 |

Frost tolerance upregulated genes (SEQ ID NO: 1-15)

| | | | | | | | |
|---|---|---|---|---|---|---|---|
| b1nr013 | −1.635 | 0.6145 | 1.7145 | 2.871 | 1.836 | 1.235 | 0.4545 |
| b1nr031 | −2.133 | 0.575 | 1.506 | 2.6795 | 2.3185 | 1.289 | 0.671 |
| b1nr039 | −1.64 | 0.8285 | 2.264 | 3.0345 | 2.181 | 1.2005 | 0.655 |
| b4nr049 | −1.369 | 0.762 | 2.074 | 2.7605 | 1.7175 | 0.829 | 0.429 |
| b4nr081 | −0.475 | 1.292 | 2.3985 | 3.267 | 2.533 | 1.9915 | 1.1565 |
| b4nr096 | −0.5885 | 1.198 | 2.4515 | 3.3535 | 2.61 | 2.089 | 1.1585 |
| b5nr012 | −1.3065 | 0.71 | 2.2365 | 3.1315 | 1.9625 | 2.17 | 0.9345 |
| b5nr018 | −2.0685 | −0.162 | 1.585 | 2.969 | 2.204 | 1.9435 | 1.5025 |
| b5nr019 | −3.786 | −0.381 | 1.753 | 3.338 | 2.658 | 2.4595 | 1.9485 |
| b5nr052 | −0.764 | 0.739 | 1.7565 | 2.709 | 1.9 | 1.7235 | 1.156 |
| b5nr078 | 0.2 | 1.44975 | 2.1715 | 3.18825 | 1.87475 | 0.59525 | 1.096 |
| b6nr008 | −1.4095 | 0.7345 | 2.234 | 2.8925 | 1.906 | 1 | 0.633 |
| b6nr046 | −0.752 | 0.636 | 1.568 | 2.896 | 2.0885 | 2.2565 | 1.3965 |
| b6nr057 | −0.5665 | 0.513 | 1.313 | 2.6415 | 2.703 | 2.8945 | 1.9005 |
| b6nr061 | −0.4095 | 0.656 | 1.5 | 2.628 | 2.486 | 2.697 | 1.7525 |

Frost tolerance downregulated genes (SEQ ID NO: 16-27)

| | | | | | | | |
|---|---|---|---|---|---|---|---|
| b1nr005 | 0.971 | 0.9845 | 0.236 | −0.389 | −3.047 | −2.796 | −2.786 |
| b1nr019 | 0.567 | 1.1315 | 0.6415 | 0.93 | −0.491 | −0.938 | −1.123 |
| b1nr025 | 1.3225 | 1.5505 | 0.749 | 0.856 | −0.837 | −1.0685 | −0.9395 |
| b1nr082 | 0.7995 | 1.2045 | 0.525 | 0.56 | −0.684 | −1.393 | −1.452 |
| b2nr070 | 0.587 | 1.0985 | 1.4655 | 1.4875 | 0.083 | 0.031 | −0.5615 |
| b2nr074 | 0.591 | 1.0015 | 1.278 | 1.4895 | −0.052 | −0.041 | −0.512 |
| b3nr031 | 0.6235 | 1.2575 | −0.008 | 0.104 | −2.7185 | −2.8945 | −3.132 |
| b3nr056 | 1.0265 | 1.042 | 0.899 | 0.8475 | −0.729 | −1.237 | −1.4325 |
| b3nr058 | 0.63725 | 1.2795 | 0.96925 | 1.0065 | −0.88975 | −1.13425 | −1.3605 |
| b3nr083 | 0.474 | 1.2695 | 1.3035 | 1.457 | 0.2995 | 0.391 | −0.259 |
| b3nr095 | 0.99433 | 1.35633 | 1.1205 | 1.2215 | −0.0895 | −0.024 | −0.463 |
| b6nr003 | −0.154 | 1.043 | 1.42 | 2.113 | 1.1115 | 0.5065 | 0.065 |

Genes with stable expression (SEQ ID NO: 28 and 29)

| | | | | | | | |
|---|---|---|---|---|---|---|---|
| b3nr038 | 0.6465 | 1.2055 | 0.8485 | 1.3285 | 0.449 | 0.463 | −0.022 |
| b4nr068 | 0.6065 | 1.4755 | 1.5845 | 2.119 | 0.7945 | 0.8845 | 0.2935 |

Some of the selected indicator genes have sequence homology to known sequences, as indicated in Table 3.

TABLE 3
Frost tolerance upregulated gene sequences

| | | | | |
|---|---|---|---|---|
| SEQ ID 1 | b1nr013 | Dehydrin | Prunus persica | 8.00E−11 |
| SEQ ID 2 | b1nr031 | Embryonic abundant protein AtEm1 | Arabidopsis | 5.00E−07 |
| SEQ ID 3 | b1nr039 | Unknown | | |
| SEQ ID 4 | b4nr049 | Unknown | | 3.2 |
| SEQ ID 5 | b4nr081 | Unknown | | 4.6 |
| SEQ ID 6 | b4nr096 | Unknown | | 0 |
| SEQ ID 7 | b5nr012 | embryonic abundant protein, 59K - | unknown origin | 7.00E−12 |
| | | soybean | | |
| SEQ ID 8 | b5nr018 | protein kinase family | [Arabidopsis thaliana] | 7.00E−27 |
| SEQ ID 9 | b5nr019 | ABA-inducible protein | [Fagus sylvatica] | 6.00E−32 |
| SEQ ID 10 | b5nr052 | PRL1 associated protein-related | [Arabidopsis thaliana] | 3.00E−39 |
| SEQ ID 11 | b5nr078 | LTCOR11 | [Lavatera thuringiaca] | 1.00E−24 |
| SEQ ID 12 | b6nr008 | Unknown | | 0.55 |
| SEQ ID 13 | b6nr046 | Unknown | | 0.024 |
| SEQ ID 14 | b6nr057 | early light-induced protein | [Arabidopsis thaliana] | 4.00E−32 |
| SEQ ID 15 | b6nr061 | probable light induced protein - | [Arabidopsis thaliana] | 2.00E−09 |
| | | Arabidopsis thaliana | | |

Frost tolerance downregulated gene sequences

| | | | | |
|---|---|---|---|---|
| SEQ ID 16 | b1nr005 | GDSL-motif lipase/hydrolase protein | Arabidopsis | 2.00E−47 |
| SEQ ID 17 | b1nr019 | arabinogalactan protein | Gossypium hirsutum | 2.00E−37 |
| SEQ ID 18 | b1nr025 | Unknown | Arabidopsis | 4.00E−23 |
| SEQ ID 19 | b1nr082 | allergenic isoflavone reductase-like | Betula pendula | 3.00E−83 |
| | | protein | | |
| SEQ ID 20 | b2nr070 | Unknown | | 0.029 |
| SEQ ID 21 | b2nr074 | Unknown | | 0.042 |
| SEQ ID 22 | b3nr031 | Unknown | | 0 |
| SEQ ID 23 | b3nr056 | expansin-related | [Arabidopsis thaliana] | 4.00E−55 |
| SEQ ID 24 | b3nr058 | alpha-tubulin | [Gossypium hirsutum] | 1.00E−62 |
| SEQ ID 25 | b3nr083 | Unknown | | 0.047 |
| SEQ ID 26 | b3nr095 | beta tubulin | [Arabidopsis thaliana] | 6.00E−67 |
| SEQ ID 27 | b6nr003 | Unknown | | 0.32 |

Constant

| | | | | |
|---|---|---|---|---|
| SEQ ID 28 | b3nr038 | protein kinase. putative | [Arabidopsis thaliana] | 4.00E−44 |
| SEQ ID 29 | b4nr068 | Unknown | | 0 |

### 1.2 Material and Methods

Expression levels can be determined in buds of tree seedlings using RT-PCR. or microarrays (described below) or any other gene expression profiling format. Results are most reliable when samples are related to a cold-sensitive sample taken in early autumn.

1.2.1 On Site. Robust Sampling

Use about 10-20 mg of plant tissue for the homogenate. Add 5 parts double distilled water to the tissue. Grind until it is apparent that some plant tissue is homogenized. The homogenate does not have to have a smooth consistency. Apply 25 microliter of plant homogenate to each circle on an FTA card (Whatman). Allow plant homogenate on FTA to dry for at least one hour at room temperature. Do not heat assist the drying period. Archive the sample in a desiccated environment.

1.2.2 RNA Isolation from Plant Homogenate on FTA Cards

Take a sample disc from the dried spot using and place it in an Eppendorf vial. Add 400 microliter RNA processing buffer (10 mM Tris-HCl, pH 8.0, 0.1 mM EDTA, 1 microliter RNAse inhibitor, 200 microgram/ml glycogen and 2 mM DTT, freshly prepared). Mix and incubate on ice for 15 minutes (mix every five minutes). Remove the disc. Precipitate the RNA with 1/10th volume of 3M sodium acetate pH 5.2 and two volumes of ice cold 100% isopropanol. Incubate for 1 hour ad −20 C. Spin down the RNA at top speed in an Eppendorf centrifuge. Wash the pellet with 75% ethanol. Air dry the pellet. Resuspend the pellet in a suitable volume of double distilled water. Use DNA free (AMBION) for removal of traces of DNA following the protocol of the manufacturer.

After that, the RNA preparation can be directly used for cDNA synthesis and subsequent PCR.

### 1.2.3 Microarray Hybridization

Total RNA, up till 20 microgram, purified with RNeasy (Qiagen, The Netherlands) and complemented with 1 nanogram luciferase polyA mRNA was used for each individual labeling. Reference RNA was labeled with Cy3 and sample RNA with Cy5 using the CyScribe First-Strand cDNA Labeling Kit (Amersham Biosciences). After checking the integrity of the labeled cDNA using agarose electrophoresis, sample and reference cDNA were mixed and used for hybridization of the microarray following the protocol supplied by the manufacturer of the slides. Cover slides and hybridization chambers from Agilent Technologies (Palo Alto) were used. Hybridization was allowed to continue overnight in an incubator where the slides were continuously rotating (Sheldon Manufacturing). Post hybridization washes were according to the Nexterion protocol.

### 1.2.4 RT-PCR

Total RNA was isolated according to the protocol described above. Preparations were DNAseI (AP Biotech) treated and purified using RNeasy (Qiagen, The Netherlands). Half a microgram of pure total RNA was used for cDNA synthesis using Anchored Oligo(dT)23 (SIGMA, The Netherlands) and M-MLV Reverse Transcriptase (Invitrogen, Life Technologies). Dilutions of this cDNA were used for Realtime PCR using the qPCR Mastermix Plus for SYBR Greenl (Eurogentec, Belgium). Product formation was measured using the iCycler system (BIORAD Laboratories, The Netherlands). Primer sets are described in SEQ ID NO:30-41. The signal obtained from the same batch of cDNA using primers homologous to Arabidopsis thaliana 18S rRNA was taken as a reference for normalization. Relative changes in expression were calculated using the Gene Expression Macro (Version 1.1) supplied by BIORAD.

### Example 2

### A Method for Determining the Ripening Stage of Pears, Exemplified by Pyrus communis L. cv Conference

### 2.1 Indicator Genes

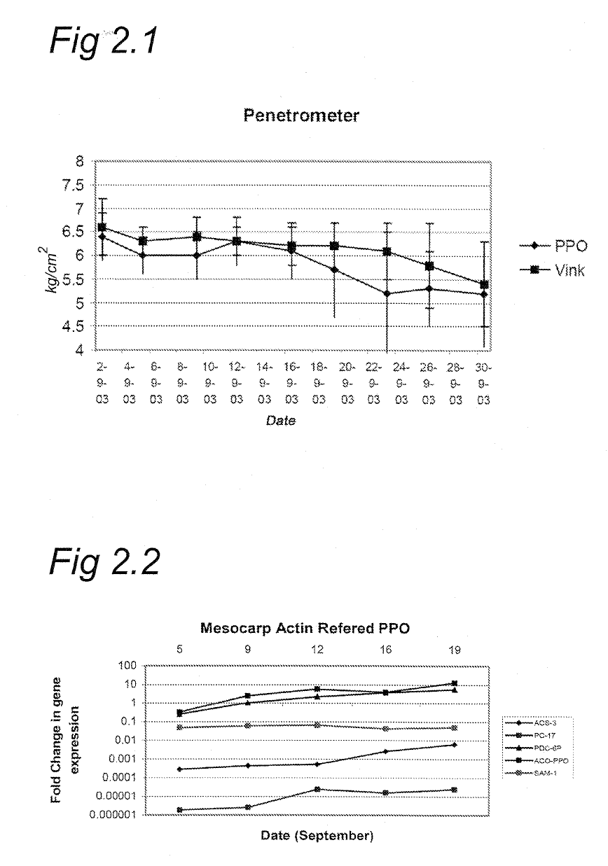

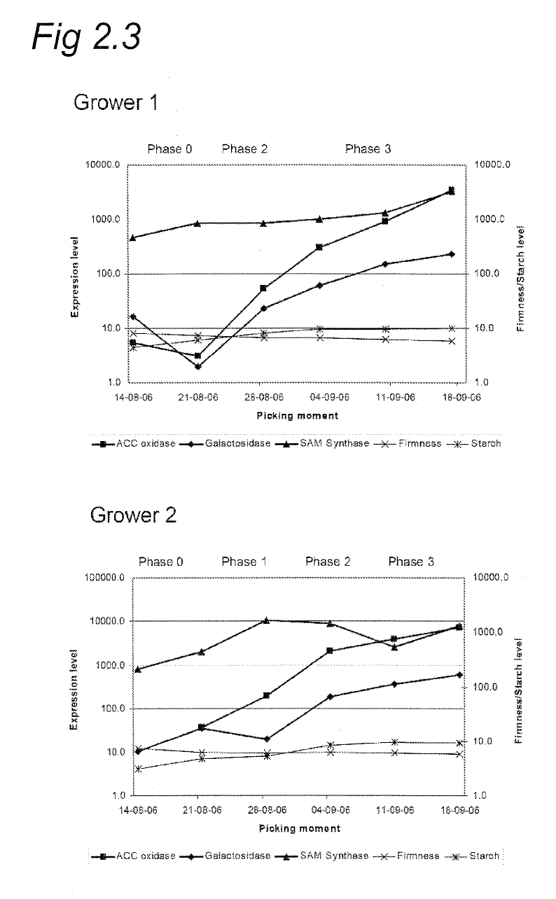

Comparison of expression levels of a set of 5 genes, SEQ ID NO:42-46, in various batches of pears gives information about relative ripening stages. This method is much more informative than firmness measurements (FIGS. 2.1 and 2.2). Discrimination between batches is possible in cases where firmness measurements fail. The data in FIGS. 2.1 and 2.2 show that during the test period the firmness hardly changes but expression of all genes, except SAM-1, increased 10 to 100 fold. The test can also be used to check the effect of storage conditions on the produce.

FIG. 2.3. shows a result of the validation of the test in practice. Based on the expression data of the indicated genes, ripening phases can be defined. This typical example shows two orchards, from two different growers, which exhibit clear differences in ripening up until 11^{th} of September. These differences in ripening behavior directly influences optimal picking date but may also have an effect on storage behavior.

Expression data of the indicator genes is shown in Table 4, below, and in FIG. 2.2.

TABLE 4

| | ACS-3 | PC-17 | PDC-6P | ACO-PPO | SAM-1 |
|---|---|---|---|---|---|
| Date (Sept.) | (SEQ ID 44) | (SEQ ID 46) | (SEQ ID 45) | (SEQ ID 43) | (SEQ ID 42) |
| 5 | 0.000281151 | 1.86E−06 | 0.247823794 | 0.321849928 | 0.047935697 |
| 9 | 0.000436517 | 2.58E−06 | 1.038177613 | 2.531683248 | 0.0596776 |
| 12 | 0.000523976 | 2.38E−05 | 2.220848325 | 5.763191619 | 0.064704058 |
| 16 | 0.002586697 | 1.58E−05 | 3.745937856 | 3.938307235 | 0.042266044 |
| 19 | 0.006349136 | 2.46E−05 | 5.587947537 | 12.75888406 | 0.051376208 |

TABLE 5 shows putative homology of indicator genes to known genes

| Sequence ID | Homology to |
|---|---|
| SEQ ID 42 | SAM synthase 1 |
| SEQ ID 43 | ACC oxidase (ACO-2) |
| SEQ ID 44 | ACC synthase (ACS3-4) |
| SEQ ID 45 | Pyruvate decarboxylase PDC-6P |
| SEQ ID 46 | (PC17) No significant homology |
| SEQ ID NO 158 | ACS1-6 |
| SEQ ID NO 159 | ACS2 (old name ACS3-6) |
| SEQ ID NO 160 | ACS6 (old name ACS4-8M) |
| SEQ ID NO 161 | ACS5-4 |
| SEQ ID NO 162 | GAPDH-7 |
| SEQ ID NO 163 | beta-galactosidase (AJ811694) |
| SEQ ID NO 164 | Polygalacturonase 1 (AJ504855.2) |
| SEQ ID NO 165 | Polygalacturonase 2 (AJ811693.1) |
| SEQ ID NO 166 | Actin (AF386514.1) |
| SEQ ID NO 167 | Beta xylosidase (AJ811690) |
| SEQ ID NO 168 | Expansin 2 (AB093029) |
| SEQ ID NO 169 | Expansin 3 (AB093030) |
| SEQ ID NO 170 | Expansin 5 (AB093032) |
| SEQ ID NO 171 | Expansin 6 (AB093033) |

### 2.2 Material and Methods

Expression levels can be determined in mesocarp of pear fruit using RT-PCR, or microarrays (described below) or any other gene expression profiling format. Results are most reliable when samples are related to an unripe sample taken well before harvest time.

### 2.2.1 On Site, Robust Sampling

Use about 10-20 mg of plant tissue for the homogenate. Add 5 parts double distilled water to the tissue. Grind until it is apparent that some plant tissue is homogenized. The homogenate does not have to have a smooth consistency. Apply 25 microliter of plant homogenate to each circle on an FTA card (Whatman). Allow plant homogenate on FTA to dry for at least one hour at room temperature. Do not heat assist the drying period. Archive the sample in a desiccated environment.

2.2.2 RNA Isolation from Plant Homogenate on FTA Cards

Take a sample disc from the dried spot using and place it in an Eppendorf vial Add 400 microliter RNA processing buffer (10 mM Tris-HC1, pH 8.0, 0.1 mM EDTA, 1 microliter RNAse inhibitor, 200 microgram/ml glycogen and 2 mM DTT, freshly prepared).

Mix and incubate on ice for 15 minutes (mix every five minutes). Remove the disc. Precipitate the RNA with 1/10th volume of 3M sodium acetate pH 5.2 and two volumes of ice cold 100% isopropanol. Incubate for 1 hour ad −20 C. Spin down the RNA at top speed in an Eppendorf centrifuge. Wash the pellet with 75% ethanol. Air dry the pellet. Resuspend the pellet in a suitable volume of double distilled water. Use DNA free (AMBION) for removal of traces of DNA following the protocol of the manufacturer.

After that, the RNA preparation can be directly used for cDNA synthesis and subsequent PCR.

### 2.2.3 Microarray Hybridization

Total RNA, up to 20 microgram, purified with RNeasy (Qiagen, The Netherlands) and complemented with 1 nanogram luciferase polyA mRNA was used for each individual labeling. Reference RNA was labeled with Cy3 and sample RNA with Cy5 using the CyScribe First-Strand cDNA Labeling Kit (Amersham Biosciences). After checking the integrity of the labeled cDNA using agarose electrophoresis, sample and reference cDNA were mixed and used for hybridization of the microarray following the protocol supplied by the manufacturer of the slides. Cover slides and hybridization chambers from Agilent Technologies (Palo Alto) were used. Hybridization was allowed to continue overnight in an incubator where the slides were continuously rotating (Sheldon Manufacturing). Post hybridization washes were according to the Nexterion protocol.

### 2.2.4 RT-PCR

Total RNA was isolated according to the protocol described above. Preparations were DNAseI (AP Biotech) treated and purified using RNeasy (Qiagen, The Netherlands). Half a microgram of pure total RNA was used for cDNA synthesis using Anchored Oligo(dT)23 (SIGMA, The Netherlands) and M-MLV Reverse Transcriptase (Invitrogen, Life Technologies). Dilutions of this cDNA were used for Realtime PCR using the qPCR Mastermix Plus for SYBR Greenl (Eurogentec, Belgium). Product formation was measured using the iCycler system (BIORAD Laboratories, The Netherlands). Primer sets are described in SEQ ID NO:47-56. The signal obtained from the same batch of cDNA using primers homologous to Arabidopsis thaliana 18S rRNA was taken as a reference for normalization. Relative changes in expression were calculated using the Gene Expression Macro (Version 1.1) supplied by BIORAD.

### Example 3

A Sensitive Method for Measuring Sensory Decay of Fruit, Exemplified by Apples

### 3.1 Indicator Genes

In an experimental approach in which transcriptional profiling (using microarrays) was combined with sensory analysis and physiological measurements a set of 20 genes was selected (SEQ ID NO:57-76) that can be used for early warning of quality decay. In the experiments quality loss was induced by storage at a temperature of 18° C., whereas normal storage temperature is 4° C.

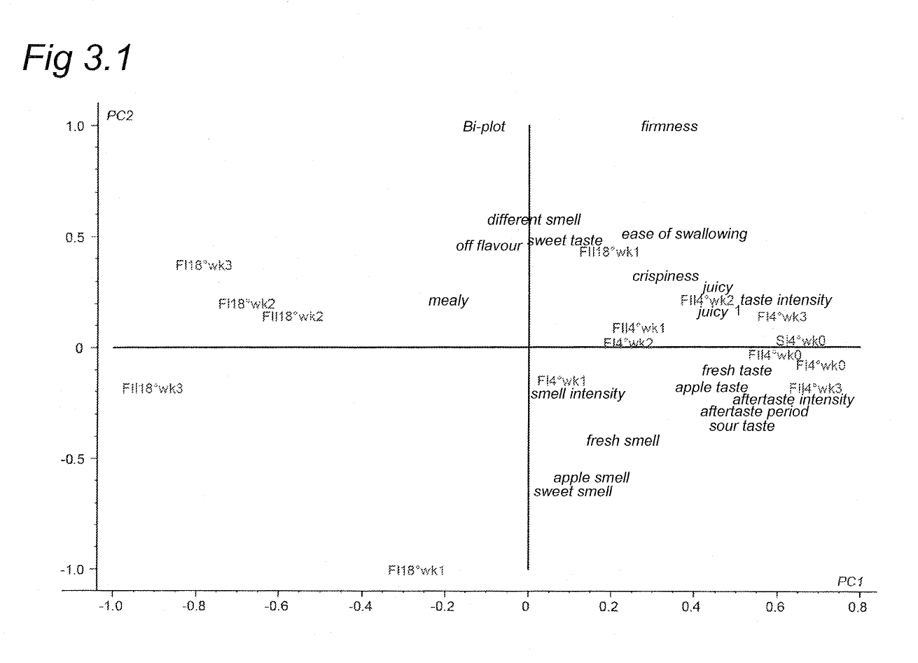

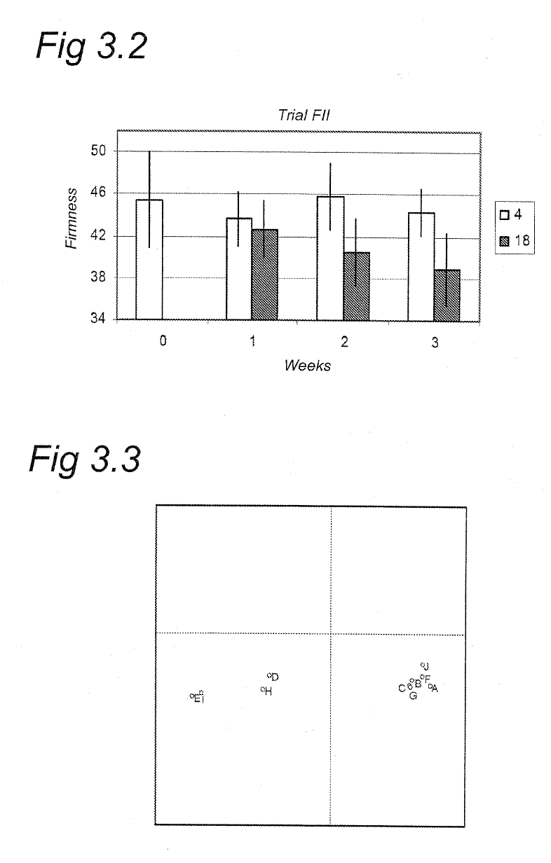

Relative expression levels of the selected genes can be used to determine whether a batch of apples is approaching a status of quality loss. Analyzing the genes provides better insight in quality loss than sensory analysis or firmness measurements. Two batches of apples were tested with the three different methods mentioned. FIG. 3.1 shows the result of the sensory analysis. From this plot it becomes clear that after one week storage at suboptimal temperature (18° C.) differences with apples stored at optimal temperature (4° C.) are hard to establish. Only after two week storage differences between the two storage conditions can be sensed.

FIG. 3.2 shows the results of the firmness measurement of one of the trials (FII). Only after two weeks storage statistical significant differences can be measured between the two storage conditions.

FIG. 3.3 shows the PCA plot of the data obtained with a microarray that contains around 900 genes. From this it is clear that already after 1 week suboptimal storage, reproducible differences can be observed. These differences were found to be explained by a very limited number of genes. A selection of genes has been made from this group.

TABLE 6
below shows the microarray expression data of indicator genes

| | | | | | | | | | | |
|---|---|---|---|---|---|---|---|---|---|---|
| ID | A | B | C | D | E | F | G | H | I | J |

Upregulated Genes as a Result of Storage at Too High Temperature, 18° C. (SEQ ID NO: 57-66)

| | | | | | | | | | | |
|---|---|---|---|---|---|---|---|---|---|---|
| Apple13nr042 | −1.25 | −0.87 | −1.15 | 0.77 | 0.94 | −1.07 | −0.58 | 0.42 | 0.53 | −0.45 |
| Apple13nr062 | −5.12 | −4.17 | −4.62 | 0.42 | 1.89 | −4.44 | −3.74 | 0.24 | 1.63 | −3.55 |
| Apple15nr090 | −1.08 | −0.74 | −1.27 | 0.31 | 0.40 | −0.72 | −0.24 | 0.35 | 0.28 | −0.21 |
| Apple5nr032 | −0.96 | −0.69 | −1.45 | 0.37 | 0.52 | −0.63 | −0.39 | 0.35 | 0.64 | −0.02 |
| Apple5nr041 | −0.84 | −0.36 | −1.04 | 0.37 | 0.79 | −0.19 | −0.29 | 0.26 | 0.62 | −0.35 |
| Apple5nr069 | −3.77 | −2.89 | −4.21 | 0.29 | 2.13 | −3.07 | −3.19 | 0.13 | 1.76 | −2.83 |
| Apple6nr013 | −1.20 | −0.59 | −1.32 | 0.84 | 1.28 | −0.73 | −1.06 | 0.64 | 0.94 | −0.69 |
| Apple6nr096 | −1.03 | −0.54 | −1.04 | 0.69 | 0.35 | −0.41 | −0.27 | 0.48 | 0.23 | −0.13 |
| Apple7nr006 | −1.27 | −1.23 | −1.96 | 1.00 | 0.29 | −1.71 | −0.94 | 0.91 | 1.35 | −0.59 |
| Apple8nr019 | −1.08 | −0.10 | −1.56 | 0.43 | 0.37 | −0.66 | −0.59 | 0.49 | 0.47 | −0.28 |

Downregulated genes as a result of storage at too high temperature, 18° C. (SEQ ID NO: 67-76)

| | | | | | | | | | | |
|---|---|---|---|---|---|---|---|---|---|---|
| Apple11nr011 | 0.27 | 0.59 | −0.12 | −2.52 | −2.79 | 0.75 | 0.11 | −2.96 | −2.22 | 1.11 |
| Apple12nr048 | 0.36 | 0.35 | −0.19 | −1.15 | −2.25 | 0.17 | 0.74 | −1.57 | −2.48 | 0.69 |
| Apple12nr056 | 0.43 | 0.40 | −0.31 | −1.08 | −2.44 | 0.12 | 0.64 | −1.50 | −2.28 | 0.62 |
| Apple12nr062 | −0.07 | 0.00 | −0.88 | −0.95 | −2.01 | 0.85 | −0.22 | −1.24 | −2.26 | 1.67 |
| Apple12nr094 | 0.37 | 0.29 | −0.10 | −1.76 | −5.11 | 0.76 | 0.29 | −2.77 | −5.54 | 1.35 |
| Apple1nr054 | 0.67 | 0.51 | −0.26 | −1.77 | −4.21 | 0.97 | 0.79 | −2.17 | −3.78 | 1.12 |
| Apple1nr089 | 0.67 | 0.18 | −0.72 | −1.25 | −1.58 | 0.99 | 0.44 | −2.03 | −1.88 | 1.42 |
| Apple2nr039 | −0.11 | 0.27 | −1.01 | −0.83 | −1.99 | 1.02 | −0.11 | −0.91 | −2.29 | 1.55 |
| Apple2nr080 | 0.29 | 0.69 | −0.27 | −1.81 | −1.77 | 0.27 | 0.37 | −1.69 | −1.37 | 1.04 |
| Apple3nr034 | −0.11 | 0.46 | −0.82 | −0.12 | −1.89 | 0.77 | 0.99 | −0.93 | −1.85 | 0.95 |

A = wk 0, 4° C. (experiment II); B = wk 1, 4° C. (experiment II); C = wk 2, 4° C. (experiment II) D = wk 1, 18° C. (experiment II); E = wk 2, 18° C. (experiment II); F = wk 0, 4° C. (experiment I) G = wk 2, 4° C. (experiment I); H = wk 1, 18° C. (experiment I); I = wk 2, 18° C. (experiment I) J = wk 0, 4° C. (experiment I)

TABLE 7 shows homology of indicator genes to known genes

Upregulated when stored at 18° C.

| | | |
|---|---|---|
| SEQ ID 57 | Apple13nr042 | SNF8 like protein (Arabidopsis thaliana) |
| SEQ ID 58 | Apple13nr062 | None |
| SEQ ID 59 | Apple15nr090 | None |
| SEQ ID 60 | Apple5nr032 | VPE-CITSI vacuolar processing |
| | | enzyme precursor |
| SEQ ID 61 | Apple5nr041 | ubiquitin conjugating protein |
| SEQ ID 62 | Apple05nr069 | ripening related gene |
| SEQ ID 63 | Apple06nr013 | Glutathione S-transferase |
| SEQ ID 64 | Apple6nr096 | sarcosine oxidase |
| SEQ ID 65 | Apple07nr006 | dicyanin (Lycopersicon esculentum) |
| SEQ ID 66 | Apple8nr019 | Lipoxygenase |

Downregulated when stored at 18° C.

| | | |
|---|---|---|
| SEQ ID 67 | Apple11nr011 | None |
| SEQ ID 68 | Apple12nr048 | vacuole associated annexin VCaB42 |
| | | (Nicotiana tabacum) |
| SEQ ID 69 | Apple12nr056 | C-methyltransferase from soybean |
| SEQ ID 70 | Apple12nr062 | endo-xyloglucan transferase from cotton |
| SEQ ID 71 | Apple12nr094 | NADP-dependent D-sorbitol- |
| | | 6-phosphate |
| SEQ ID 72 | Apple01nr054 | None |
| SEQ ID 73 | Apple1nr089 | None |
| SEQ ID 74 | Apple2nr039 | xyloglucan endo-transglycolase-like |
| | | protein |
| SEQ ID 75 | Apple2nr080 | heat shock protein 70 |
| | | (Arabidopsis thaliana) |
| SEQ ID 76 | Apple3nr034 | None |

### 3.2 Material and Methods

Expression levels were determined in mesocarp of apple fruit using microarrays (described below) or any other gene expression profiling format, such as RT-PCR. Results are most reliable when samples are related to an sample taken at harvest time.

### 3.2.1 On Site, Robust Sampling

Use about 10-20 mg of plant tissue for the homogenate. Add 5 parts double distilled water to the tissue. Grind until it is apparent that some plant tissue is homogenized. The homogenate does not have to have a smooth consistency. Apply 25 microliter of plant homogenate to each circle on an FTA card (Whatman).

Allow plant homogenate on FTA to dry for at least one hour at room temperature. Do not heat assist the drying period. Archive the sample in a desiccated environment.

3.2.2 RNA Isolation from Plant Homogenate on FTA Cards

Take a sample disc from the dried spot using and place it in an Eppendorf vial

Add 400 microliter RNA processing buffer (10 mM Tris-HC1, pH 8.0, 0.1 mM EDTA, 1 microliter RNAse inhibitor, 200 microgram/ml glycogen and 2 mM DTT, freshly prepared). Mix and incubate on ice for 15 minutes (mix every five minutes). Remove the disc. Precipitate the RNA with 1/10th volume of 3M sodium acetate pH 5.2 and two volumes of ice cold 100% isopropanol. Incubate for 1 hour ad −20 C. Spin down the RNA at top speed in an Eppendorf centrifuge. Wash the pellet with 75% ethanol. Air dry the pellet. Resuspend the pellet in a suitable volume of double distilled water. Use DNA free (AMBION) for removal of traces of DNA following the protocol of the manufacturer. After that, the RNA preparation can be directly used for cDNA synthesis and subsequent PCR.

### 3.2.3 Microarray Hybridization

Total RNA, up till 20 microgram, purified with RNeasy (Qiagen, The Netherlands) and complemented with 1 nanogram luciferase polyA mRNA was used for each individual labeling. Reference RNA was labeled with Cy3 and sample RNA with Cy5 using the CyScribe First-Strand cDNA Labeling Kit (Amersham Biosciences). After checking the integrity of the labeled cDNA using agarose electrophoresis, sample and reference cDNA were mixed and used for hybridization of the microarray following the protocol supplied by the manufacturer of the slides. Cover slides and hybridization chambers from Agilent Technologies (Palo Alto) were used. Hybridization was allowed to continue overnight in an incubator where the slides were continuously rotating (Sheldon Manufacturing). Post hybridization washes were according to the Nexterion protocol.

### Example 4

### Prediction Greymold (Botrytis cinerea) in Cut Flowers, Exemplified in Rose (Rosa hybrida L. cv. Bianca)

### 4.1 Indicator Genes

A set of 36 indicator genes (SEQ ID NO:77-109 and SEQ ID NO:172-174) have been selected whose expression profile can be used as measurement to predict the susceptibility of roses to Botrytis.

Based on the expression level of the described genes conclusions can be drawn about the predicted quality class of the batch of roses (good-almost no Botrytis, moderate-some Botrytis disease will develop or bad-severe Botrytis disease can be expected). The genes have been selected based on 12 batches of Bianca roses with different levels of Botrytis decay after 1 week of vase-life. Evaluation of gene expression and prediction using the indicator genes has been performed using Real Time PCR analysis of the same 12 batches, normalized using the housekeeping genes listed in SEQ ID NO:110-112.

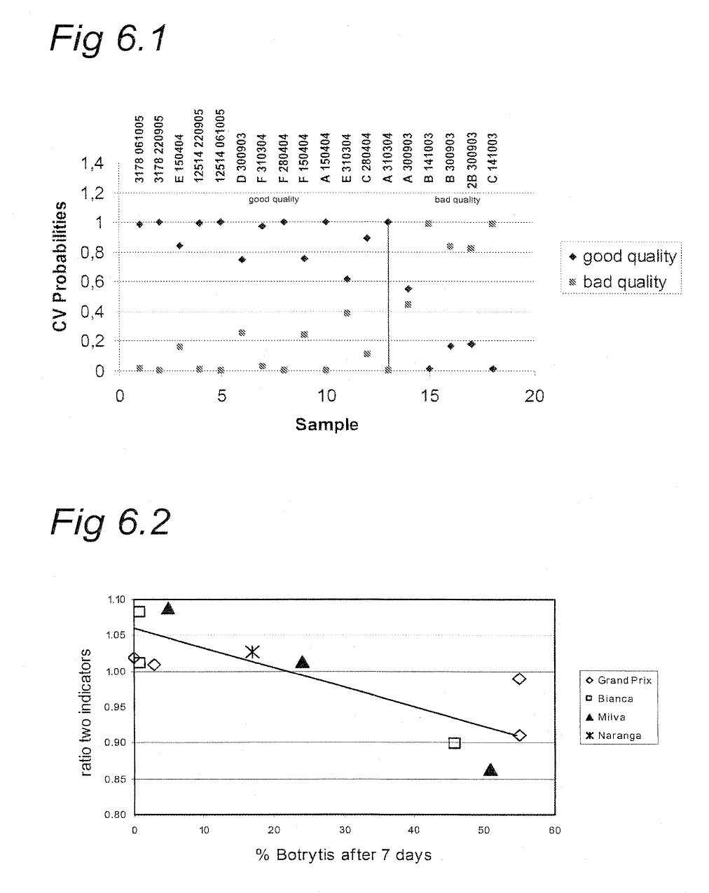

FIG. 6.1 shows a typical result where expression data of 22 genes (marked in Table 8 below) were used to classify two quality groups. High probability (1) indicates a reliable prediction. From the Figure it is clear that in most cases quality prediction is reliable (probability above 0.8).

FIG. 6.2 shows a graph where the expression ratio of only two genes was plotted against the percentage of decay as a result of Botrytis invasion. It indicates that the expression levels of small number of genes, whether or not after mathematical conversion, also gives a good prediction.

TABLE 8 shows homologies of indicator genes from rose to known genes

| | | Putative function based | Used in |
|---|---|---|---|
| SEQ ID NO: | Name | on homology | FIG. 6.1 |
| SEQ ID 96 | OProseR0008 | Unknown | |
| SEQ ID 97 | OProseR0053 | putative xyloglucan | X |
| | | endotransglycosylase | |
| SEQ ID 98 | OProseR0060 | endo-xyloglucan transferase | X |
| SEQ ID 99 | OProseR0106 | phosphate transport protein | |
| | | (probably Botrytis cinerea) | |
| SEQ ID 100 | OProseR0238 | putative lipid transfer protein | X |
| SEQ ID 101 | OProseR0260 | Unknown | |
| SEQ ID 102 | OProseR0277 | Unknown | X |
| SEQ ID 103 | OProseR0286 | Protein disulfide isomerases | X |
| SEQ ID 104 | OProseR0371 | Unknown | X |
| SEQ ID 105 | OProseR0556 | Unknown | X |
| SEQ ID 106 | OProseR0626 | Unknown | X |
| SEQ ID 107 | OProseR0763 | polygalacturonase inhibitor | X |
| | | protein | |
| SEQ ID 108 | OProseR0774 | aquaporin protein | X |
| SEQ ID 109 | OProseR0812 | S-adenosyl-L-methionine | X |
| | | decarboxylase | |
| SEQ ID 77 | OProseR1069 | glutathione S-conjugate | |
| | | transporting ATPase | |
| SEQ ID 78 | OProseR1072 | Unknown | X |
| SEQ ID 79 | OProseR1093 | Unknown | X |
| SEQ ID 80 | OProseR1094 | Vacuolar ATP synthase 16 | |
| | | kDa proteolipid subunit | |
| SEQ ID 81 | OProseR1100 | chalcone synthase | X |
| SEQ ID 82 | OProseR1117 | amygdalin hydrolase isoform | X |
| | | AH I precursor | |
| SEQ ID 83 | OProseR1198 | delta 9 acyl-lipid desaturase | |
| SEQ ID 84 | OProseR1208 | Unknown | |
| SEQ ID 85 | OProseR1246 | Unknown | |
| SEQ ID 86 | OProseR1322 | mitochondrial formate | X |
| | | dehydrogenase precursor | |
| SEQ ID 87 | OProseR1391 | Unknown | |
| SEQ ID 88 | OProseR1459 | translation initiation factor IF1 | |
| SEQ ID 89 | OProseR1481 | Unknown | X |
| SEQ ID 90 | OProseR1674 | Probable NADH-ubiquinone | X |
| | | oxidoreductase | |
| SEQ ID 91 | OProseR1700 | Unknown | |
| SEQ ID 92 | OProseR1727 | 3-hydroxy-3-methylglutaryl | |
| | | coenzyme A reductase | |
| SEQ ID 93 | OProseR1783 | GAST-like gene product | X |
| SEQ ID 94 | OProseR1792 | Unknown | |
| SEQ ID 95 | OProseR1807 | putative proteasome alpha | |
| | | subunit | |
| SEQ ID 172 | OProseR1663 | HHG4 nucleoid DNA-binding | X |
| | | protein | |
| SEQ ID 173 | OProseR0948 | Unknown | X |
| SEQ ID 174 | OProseR0049 | Actin | X |

### 4.2 Material and Methods

### 4.2.1 Sampling

First three outer petals of 25 roses have been used for development of the test. The remaining 75 roses of the same batch were used to determine the % of flowers showing Botrytis disease after a 7 days incubation period at 21° C., 60% RH using standard light regime of 10 h light, 14 hours darkness. The 25×3 outer petals were frozen directly in liquid nitrogen, powered using mortal and pestle and stored at −80° C.

### 4.2.2 RNA Isolation

As described above using RNA easy. Concentration can be determined spectrophotometrically or using nanodrop apparatus.

### 4.2.3. Microarray Hybridization

Hybridizations have been performed using a indirect labeling protocol.

### 4.2.4. Statistical Analysis

Genes have been selected using T-tests and the software Significant Analysis of Microarray-(http://www-stat.stanford.edu/˜tibs/SAM), profile analysis using the program Spotfire (http://www.spotfire.com/products/decisionsite_microarray_analysis.cfm) in which Botrytis disease % were correlated to genes expression profiles, and using the software Predicted Analysis Microarray (http://www-stat.stanford.edu/˜tibs/PAM).

### 4.2.5. Primer Development

Primers for the selected genes were designed using Primer 3 software (http://frodo.wi.mit.edu/cgi-bin/primer3/primer3_www.cgi) in combination with DNA mfold software (http://www.bioinfo.rpi.edu/applications/mfold/old/dna/).

### 4.2.6 Real Time RT PCR

Reverse Transciptase reaction using oligo dT and Real Time analysis using the diluted cDNA can be used in a standard RealTime PCR protocol.

### Example 5

### Discoloration of Mushrooms, Exemplified by White Button Mushroom (Agaricus bisporus) Fruiting Body

### 5.1 Indicator Genes

Based on two batches white button mushrooms of the same development stage but with different degrees of browning a suppressive subtraction hybridization library has been constructed. Out of the cloned and sequenced clones 878 clones were selected for printing on the microarray, together with 19 clones from literature (Eastwood, D C et al. 2001, Genes with increased transcript levels following harvest of the sporophores of Agaricus bisporus have multiple physiological roles. Mycol. Res. 105:1223-1230). Also a partial sequence of the published polyphenol oxidase sequence PPO1 (Wichers, H J et al., 2003, Appl. Microbiol. Biotechnol. 61:336-341) was printed on the same array.

Samples with different storage quality were selected form a large range of samples that we randomly collected from growers in the Netherlands. The representative sample of 250 g mushrooms were taken from a batch of 2 kg. The 250 g was frozen in liquid nitrogen, the remaining mushrooms were analyzed for color using computer image analysis at day of sampling and after 7 days storage at 2° C.

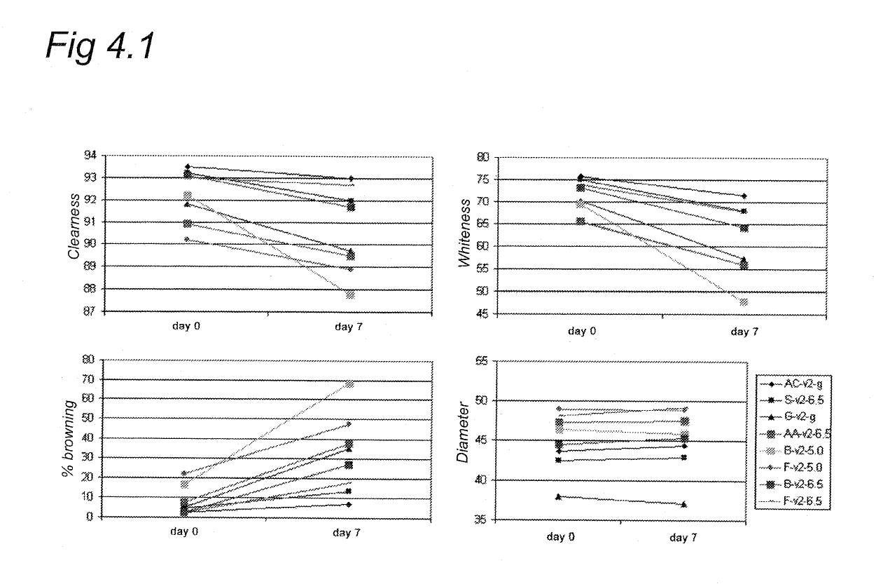

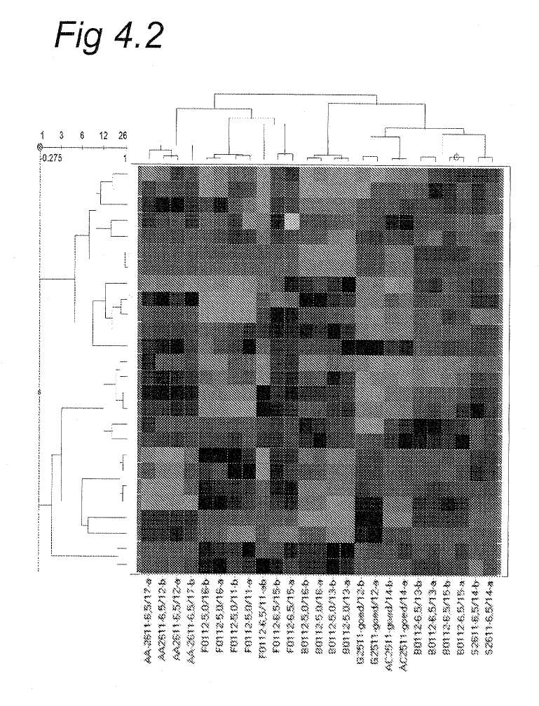

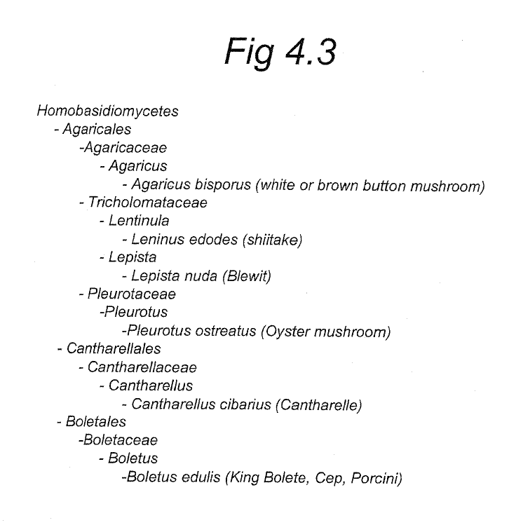

The microarrays were hybridized with 8 samples (see FIG. 4.1) of which two (AC2511 en G2511) were harvested stored overnight by 4° C. and then sampled. The other six were harvested at the same day as they were sampled with a night cold storage. The letters indicate the grower, so two batches with different storage quality of the same grower delivered the same day were obtained from grower B and grower F.

Using the same selection tools t-tests, SAM, profile correlation with browning after 7 days, hierarchical clustering, PAM a selection of putative indicator genes was generated (see SEQ ID NO:113-135 and FIG. 4.2). For some of the genes expression profiles were validated by real Time PCR analysis, normalized by 18S analysis, which fitted in almost al cases with microarray based gene expression.

TABLE 9 shows homology of indicator genes to known genes, indicating putative function

| | | Putative function based on |
|---|---|---|
| SEQ ID NO: | Name | homology |
| SEQ ID 113 | AJ271698 | Unknown |
| SEQ ID 114 | AJ271702 | Unknown |
| SEQ ID 115 | AJ271701 | Unknown |
| SEQ ID 116 | AJ271693 | B-(1-6) glucan synthase |
| SEQ ID 117 | AJ271707 | cytochrome P450 |
| SEQ ID 118 | AJ271696 | Involved in DNA binding and repair |
| SEQ ID 119 | X85113 | Polyphenoloxidase |
| SEQ ID 120 | SSH03nr023 | unknown |
| SEQ ID 121 | SSH05nr012 | unknown |
| SEQ ID 122 | SSH05nr019 | unknown |
| SEQ ID 123 | SSH07nr010 | Unknown |
| SEQ ID 124 | SSH07nr032 | Unknown |
| SEQ ID 125 | SSH07nr041 | Unknown |
| SEQ ID 126 | SSH07nr083 | endochitinase |
| SEQ ID 127 | SSH09nr088 | Urease |
| SEQ ID 128 | SSH10nr021 | Unknown |
| SEQ ID 129 | SSH10nr028 | Unknown |
| SEQ ID 130 | SSH10nr080 | unknown |
| SEQ ID 131 | SSH11nr017 | Putative sugar transporter |
| SEQ ID 132 | SSH12nr023 | Unknown |
| SEQ ID 133 | SSH12nr053 | myosin heavy chain A |
| SEQ ID 134 | SSH13nr021 | Unknown |
| SEQ ID 135 | SSH13nr086 | Unknown |

### 5.2 Material and Methods

### 5.2.1 Sampling

Stipes of mushrooms were cut off at the based of the cap. The cap was sliced and directly frozen in liquid nitrogen and stored at −80 C. Frozen, sliced caps were powdered in a blender while continuously chilled using liquid nitrogen.

### 5.2.2 RNA Isolation

RNA was isolated using RNAeasy, including a shredder homogenizing and clarification step. Concentration can be determined spectrophotometrically or using nanodrop apparatus.

### 5.2.3. Microarray Hybridization

Hybridizations have been performed using a indirect labeling protocol.

5.2.4. Statistical analysis

Genes have been selected using T-tests and the software Significant Analysis of Microarray-(http://www-stat.stanford.edu/˜tibs/SAM), profile analysis using the program Spotfire (http://www.spotfire.com/products/decisionsite_microarray_analysis.cfm) in which clearness, witness and % browning were correlated to gene expression profiles, and using the software Predicted Analysis Microarray (http://www-stat.stanford.edu/˜tibs/PAM).

5.2.5 Primer generation

Primers for the selected genes were designed using Primer 3 software (http://frodo.wi.mit.edu/cgi-bin/primer3/primer3_www.cgi) in combination with DNA mfold software (http://www.bioinfo.rpi.edu/applications/mfold/old/dna/).

### 5.2.6 Real Time RT PCR

Reverse Transcriptase reaction using oligo dT and Real Time analysis using the diluted cDNA can be used in a standard RealTime PCR protocol.

5.3 Species The technique has been developed on Agricus bisporus strain U1 and A15 and is (because genetic variation is very limited between cultivated strains of Agaricus bisporus) also applicable for other strain (brown, portobello) of the same species. Also other species of Agaricus can be diagnosed using this method. Homologous genes of the genes listed in the table above may also be applicable for quality diagnostic of other edible mushrooms like shiitake (Lentinus edodes), Pleurotus ostreatus, the Oyster mushroom and Lepista nuda (synonyms Clitocybe nuda, Tricholoma nudum en Rhodopaxillus nudus) which are close relatives of Agaricus bipsorus or even more distantly related edible mushrooms like Cantharellus cibarius and Boletus edulis (see FIG. 4.3).

### Example 6

### Prediction of Postharvest Firmness Development in Solanaceous Fruits, Exemplified in Tomato (Solanum lycopersicum L. cv. Aromata)

### 6.1 Indicator Genes

A set of 19 indicator genes (SEQ ID NO:136-154) have been selected whose expression profile can be used as measurement to predict the rate at which harvested Solanaceous fruits loose their firmness.

Based on the expression level of the described genes conclusions can be drawn about the predicted quality class of the batch of fleshy fruit such as tomatoes (good-firmness retained for more than 28 days, moderate-firmness retained for more than 15 days or bad-firmness drops below acceptable levels within 15 days). The genes have been selected based on 44 batches of Aromata tomatoes with different levels of firmness development during 6 weeks of post-harvest storage. Evaluation of gene expression and prediction using the indicator genes has been performed using microarray analysis of a subset of 16 batches, normalized using the housekeeping genes listed in SEQ ID NO:155-157.

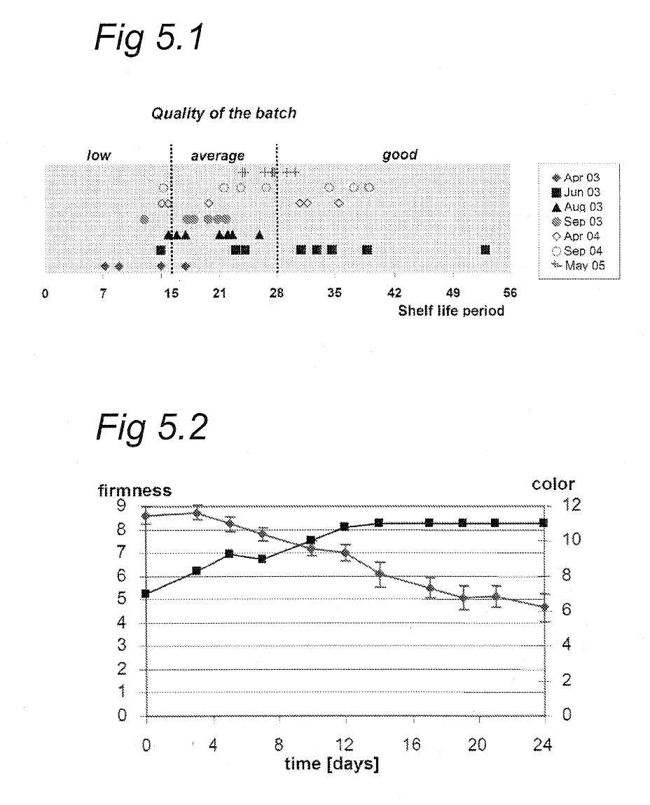

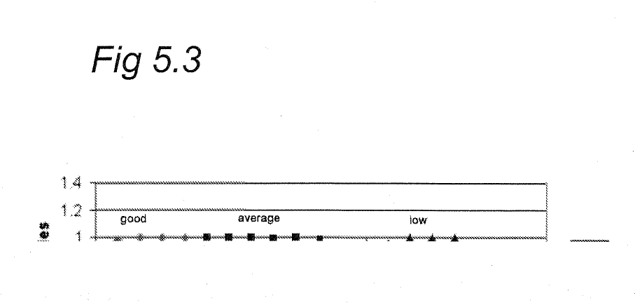

As shown in FIG. 5.3 the 19 indicator genes are able to discriminate between the 3 quality classes indicated before.

TABLE 10 shows homologies of indicator genes from tomato to known genes:

| SEQ | | | |
|---|---|---|---|
| ID | | Putative function based | |
| NO: | Name | on homology | E value |
| 136 | Tomaat1Fnr092 | Fruit-specific protein - tomato | 4.00E−46 |
| 137 | Tomaat1Fnr176 | Unknown | 7.00E−04 |
| 145 | Tomaat1Fnr224 | Unknown | 3.00E−11 |
| 146 | Tomaat3Fnr100 | protein kinase | 2.00E−05 |
| 138 | Tomaat3Fnr355 | Unknown | 0.00E+00 |
| 147 | Tomaat7Rnr298 | pyruvate decarboxylase | 3.00E−15 |
| 148 | Tomaat7Rnr310 | Unknown | 6.00E−84 |
| 149 | Tomaat7Rnr355 | malate dehydrogenase | 3.00E−69 |
| 150 | Tomaat7Rnr478 | Alcohol dehydrogenase | 3.00E−12 |
| 139 | Tomaat7Rnr558 | ATPase β subunit | 1.00E−23 |
| 151 | Tomaat7Rnr563 | Cytochrome P450-dependent | 3.00E−66 |
| | | fatty acid hydroxylase | |
| 152 | Tomaat7Rnr567 | Unknown | 2.00E−03 |
| 153 | Tomaat9Fnr019 | DNA binding protein | 3.00E+00 |
| 140 | Tomaat9Fnr046 | Unknown | 0.00E+00 |
| 141 | Tomaat9Fnr216 | Ribulose bisphosphate carboxylase | 1.00E−32 |
| | | small subunit 2A | |
| 142 | Tomaat9Fnr244 | Pathogenesis-related protein STH-2 | 2.00E−83 |
| 154 | Tomaat9Fnr248 | sucrose transport protein | 1.00E−45 |
| 143 | Tomaat9Fnr323 | chitinase | 2.00E−17 |
| 144 | Tomaat9Fnr349 | two-component sensor histidine | 2.00E−19 |
| | | kinase | |

TABLE 11 shows additional genes which have a constant expression and are suitable for normalization:

| SEQ ID NO | Name | Putative function | E value |
|---|---|---|---|
| SEQ ID 155 | Tomaat3Fnr244 | Unknown protein | 1.00E−63 |
| | | Arabidopsis Genbank | |
| | | NP_566171 | |
| SEQ ID 156 | Tomaat3Fnr268 | GTP-binding nuclear | 8.00E−35 |
| | | protein Ran1 GenBank | |
| | | P38546 | |
| SEQ ID 157 | Genbank L08255 | Tomato abscisic stress | |
| | | ripening protein 1 | |

### 6.2 Material and Methods

### 6.2.1 Sampling

Tomatoes (variety Aromata) were obtained from different growers and moved to the laboratory 1 day after picking Pericarp slices (2 from each fruit, 12 or more fruits per batch) spanning approximately 1 cm² of surface and the entire pericarp thickness were taken from the equatorial region of each fruit. Slices from the same batch were pooled and stored at −80° C. until further use.

### 6.2.2 RNA Isolation

For mRNA isolation 4 g of frozen material was ground in liquid nitrogen and transferred to an RNAse free centrifuge tube. To this 25 ml of lysis buffer (100 mM Tris HCl pH7.5, 500 mM LiCl, 10 mM EDTA, 1% lithium dodecylsulfate, 5 mM dithiothreitol) was added and homogenized by vortexing. After incubation for 5 min at 65° C. the tuber was centrifuged and supernatant transferred to a clean tube. 200 μl of washed oligo-dT-conjugated Dynabeads were added and incubated for 60 min on roller bench. Beads were isolated using a magnet and RNA was washed and eluted according to the manufacturer's instructions. Eluted mRNA underwent an extra purification round by binding to Dynabeads and subsequent elution. Concentration was determined spectrophotometrically using nanodrop apparatus.

### 6.2.3. Microarray Hybridization

mRNA was purified as described above. 2.5 μg of poly (A⁺) RNA was spiked with 1.0 ng of in vitro synthesized luciferase mRNA (Promega) and reverse transcribed in the presence of 5-(3-aminoallyl)-2′-dUTP (Sigma A0410) using 2 μg oligo (dT)₂₁ as a primer. A 25 μL reaction containing, in addition to the oligo (dT)-annealed RNA template, 1× first strand buffer (Life Technologies), 10 mM DTT, 15 U ribonuclease inhibitor (Life Technologies), 0.5 mM dATP, 0.5 mM dCTP, 0.5 mM dGTP, 0.3 mM dTTP, 0.2 mM aminoallyl-dUTP and 150 U SuperScript II RNase H-reverse transcriptase (Life Technologies) was incubated at 37° C. for 2 hr. Nucleic acids were then ethanol precipitated at room temperature and dissolved in 10 μL, 1×TE (pH 8.0). Next, cDNA/mRNA hybrids were denatured (3 min at 98° C.) and chilled on ice. RNA was degraded by adding 2.5 μL, 1 M NaOH and incubating 10 min at 37° C. After neutralizing the mixture by adding 2.5 μL, 1M HEPES (pH 6.8) and 2.0 μL, 1 M HC1, the cDNA was recovered by ethanol precipitation and resuspended in 10.0 μL, 0.1 M sodium carbonate buffer (pH 9.3). In a second step the modified cDNA was coupled to a fluorescent dye, either Cyanine 3 (Cy3) or Cyanine 5 (Cy5), using reactive Cy3- or Cy5-NHS-esters (Amersham Pharmacia). To this end 10.0 μL, of a 10 mM dye solution in DMSO was added to 10.0 μL, of the cDNA sample and incubated at room temperature for 30 min. Finally, the labeled cDNA was ethanol precipitated twice and dissolved in 5 μL, MQ.

Following prehybridization at 42° C. for 2 hr in a few ml of hybridization buffer (50% formamide, 5×Denhardt's reagent, 5×SSC, 0.2% SDS, 0.1 mg/ml denatured fish DNA), slides were rinsed in MQ and in isopropanol and then dried by centrifugation (1 min, 470×g). For a dual hybridization, 35 μL, of hybridization mixture, containing both (Cy3- and Cy5-labelled) samples at a concentration corresponding to 8 ng of the initial mRNA per μL, mixture, was used. Prior to use, the hybridization mixture was heated at 95° C. (1 min), cooled on ice and spun down to remove any debris. Hybridizations were done over night at 42° C. using a Gene Frame (10×10 mm, 25 μL, volume; ABgene) in a hybridization chamber. After hybridization, slides were washed at room temperature in 1×SSC, 0.1% SDS (5 min) followed by 0.1×SSC, 0.1% SDS (5 min) and rinsed briefly in 0.1×SSC before drying by centrifugation (1 min, 470×g).

Microarray slides were scanned with a ScanArray 3000 (Packard BioScience) with 75% laser power and 75% attenuation at a resolution of 10 μm. The resulting Cy3 and Cy5 images were stored as TIFF-files. Total pixel intensities within a fixed area (circle, ø12 pixels) were obtained for each spot using ArrayVision image analysis software (Imaging Research). Fluorescence data were imported in a spreadsheet for further work. Background spot fluorescence was determined as the mean of the fluorescence of designated spots and subtracted for each channel.

### 6.2.4. Statistical Analysis

Genes have been selected using GeneMaths 2.1 software ((Applied Maths, Sint-Martens-Latem, Belgium) in which rate of firmness loss was correlated to genes expression profiles, and using the software Predicted Analysis Microarray (http://www-stat.stanford.edu/˜tibs/PAM).

### 6.2.5. Primer Development

Primers for the selected genes were designed using Primer Express 1.0 software (Applied Biosystems).

### 6.2.6 Real Time RT PCR

Reverse Transciptase reaction using oligo dT and Real Time analysis using the diluted cDNA was performed with standard RealTime PCR protocol utilizing one step SYBR green mastermix for qPCR (Eurogentec) on a ABI Prism 7700 sequence detection system (Applied Biotechnologies).

### 6.2.7 Determination of Firmness

Tomato firmness was measured on a representative selection of 15 tomatoes per batch per sample moment. Firmness was determined using sensoric measurements or an Instron firmness tester. The Instron firmness test measures the impression of the fruits upon pressure of a plunger that is applied with a force of 3 N (non-destructive). Both measurements correlate high in our lab (r>0.91), this correlation was previously established by Kader et al. (1978, J. Amer. Soc. Hort. Sci 103:70-73) and Polderdijk et al. (1993, Postharvest Biol. Technol. 2:179-85).

In the test sensoric measure values are used. These correlate with Instron values as depicted in the TABLE 12 below:

TABLE 12

| Sensoric | Instron value | Consumer |
|---|---|---|
| value | (mm) | validation |
| 2 | −1.3 | Very Soft |
| 3 | −1.1 | Soft |
| 4 | −0.75 | Fairly firm |
| 5 | −0.6 | Firm |
| 6 | −0.4 | Very Firm |
| 7 | −0.2 | Hard |
| 8 | 0 | Extra Hard |

For the experiments the value of 5 was determined as the lowest firmness level that is still acceptable for consumers.

What is claimed is:

1. A method for determining a future quality trait stage of one or more plants, plant parts, edible mushrooms or edible mushroom parts, comprising:
(a) determining an expression profile of a set of at least two indicator mRNA transcripts in nucleic acid samples from one or more plants, mushrooms, plant parts or mushroom parts, which expression profile is indicative of a future quality trait stage,
(b) identifying the one or more plants, mushrooms, plant parts or mushroom parts, that exhibit a desired expression profile of the indicator mRNA transcripts
thereby determining said future quality trait stage.

2. A method of preparing a selected population of plants, edible mushrooms, plant parts, or edible mushrooms parts on the basis of a future quality trait stage, comprising:
(a) determining the expression profile of said set of at least two indicator mRNA transcripts in accordance with claim 1; and
(b) separating from one another said one or more plants, mushrooms, plant parts or mushroom parts that exhibit a selected expression profile
thereby preparing said selected population.

3. A method of preparing a selected population of plants, edible mushrooms, plant parts, or edible mushrooms parts on the basis of a future quality trait stage, comprising:
(a) determining an expression profile of a set of at least two indicator mRNA transcripts in nucleic acid samples from one or more plants, edible mushrooms, edible plant parts or edible mushroom parts, which expression profile is indicative of a future quality trait stage,
(b) identifying and selecting the one or more plants, mushrooms, plant parts or mushroom parts, that exhibit a selected future quality trait stage as indicated by said expression profile of said indicator mRNA transcripts determined in step (a), and
(c) separating the one or more plant, edible mushrooms, plant parts or mushroom parts having the selected future quality trait stage,
thereby preparing said selected population of plants, edible mushrooms, plant parts or mushroom parts.

4. The method according to claim 1, wherein the plant is of a family selected from the group consisting of Fagaceae, Maloideae, Rosoideae, Solanaceae and wherein the edible mushroom is a basidiomycete.

5. The method according to claim 1, wherein the future quality trait is a physiological trait or a morphological characteristic.

6. The method according to claim 3, wherein, said plants, plant parts, mushrooms or mushroom parts are separated into batches in step (c), and wherein for each batch, a nucleic acid sample comprising nucleic acids obtained from at least 5 different plants or mushrooms of the batch is analyzed in steps (a) and (b).

7. The method according to claim 1, wherein the nucleic acid sample of step (a) is obtained from tissue homogenate and the mRNA is stabilized on a solid carrier.

8. The method according to claim 1, wherein the expression profile of at least 3, different indicator mRNA transcripts is determined.

9. The method according to claim 1, wherein the expression profile of at least 4 different indicator mRNA transcripts is determined.

10. The method according to claim 1, wherein the expression profile of at least 5 different indicator mRNA transcripts is determined.

11. A solid carrier comprising at least 3 bound nucleic acid molecules that are capable of hybridizing to at least 3 indicator mRNA transcripts indicative of a future quality trait stage of one or more plants or edible mushrooms.

12. The carrier according to claim 11 comprising at least 4 of said bound nucleic acid molecules, capable of hybridizing to at least 4 of said indicator mRNA transcripts.

13. The carrier according to claim 11 comprising at least 5 of said bound nucleic acid molecules, capable of hybridizing to at least 5 of said indicator mRNA transcripts.

14. The carrier according to claim 11, which is made of glass, plastic, nitrocellulose, nylon or silicon.

15. A kit for determining the future quality trait stage of a plant or mushroom sample, comprising nucleic acid probes or primers capable of detecting the presence and/or quantity of at least 3 nucleic acid molecules of a set of indicator mRNA transcripts indicative of a future quality trait stage.

16. The kit according to claim 15 capable of detecting the presence and/or quantity of at least 4 nucleic acid molecules of a set of indicator mRNA transcripts indicative of a future quality trait stage.

17. The kit according to claim 15 capable of detecting the presence and/or quantity of at least 5 nucleic acid molecules of a set of indicator mRNA transcripts indicative of a future quality trait stage.

18. The kit according to claim 15, further comprising one or more of the following: instructions for use, control samples, control data, labeling reagents, detection reagents, hybridization or amplification reagents, primers or probes for detecting housekeeping-gene transcripts, a container or carrier

19. The kit according to claim 18 wherein the carrier comprises at least 3 bound nucleic acid molecules, said molecules being capable of hybridizing to at least 3 indicator mRNA transcripts indicative of a future quality trait stage.

20. The kit according to claim 15, further comprising a material for sampling plant or mushroom tissue.
